(19) United States
(12) Patent Application Publication
(10) Pub. No.: US 2008/0312430 A1
(43) Pub. Date: Dec. 18, 2008

(54) Method of Preparing or Synthesizing Polyazamacrocycle Derivatives

(75) Inventors: Simon Cihelnik, Krupka (CZ); Ladislav Droz, Kladno (CZ); Martin Sramek, Kladno (CZ)

Correspondence Address: HENRY M FEIEREISEN, LLC;HENRY M FEIEREISEN, 708 THIRD AVENUE, SUITE 1501, NEW YORK, NY, 10017, US

(73) Assignee: THERAPHARM GMBH, Zug (CH)

(21) Appl. No.: 11/577,561

(22) PCT Filed: Oct. 20, 2005
(86) PCT No.: PCT/EP05/11319

§ 371 Date: Mar. 4, 2008

(30) Foreign Application Priority Data
Oct. 20, 2004 (EP) 04024981.5

(51) Int. Cl. C07D 257/02

(52) U.S. Cl. 540/474

(57) ABSTRACT

The present invention relates to novel processes for the synthesis of polyazamacrocycle derivatives. Furthermore, the present invention relates to novel polyazamacrocycle derivatives as well as novel intermediates for the synthesis of said polyazamacrocycle derivatives.

### FIELD OF THE INVENTION

The present invention relates to novel processes for the synthesis of polyazamacrocycle derivatives. Furthermore, the present invention relates to novel polyazamacrocycle derivatives as well as novel intermediates for the synthesis of said polyazamacrocycle derivatives.

### BACKGROUND OF THE INVENTION

Binding of biologically active molecules and supramolecules to ionophores, general chelators or other inclusion compounds have great application potential, e.g. as radioimunopharmaceuticals, radiodiagnosticals, general diagnosticals, specific transport agents etc. (G. Hermanson—Bioconjugate Techniques., Academic Press; 1st edition 1996; V. C. Wilhelm—Immunoconjugates Antibody Conjugates in Radioimaging and Therapy of Cancer. Oxford, University Press, 1987). The role of ionophores in these supramolecular structures is evident: to complexate cation, anion or nonionic molecule. Each ionophore disposes in an exact chemical situation by exact physico-chemical parameters as constant stability of complex, reaction rate of complexation, photochemical or physiological degradability rate etc. It is possible to convert these parameters to some pseudoparameters with more predicative ability as complexation selectivity, physiological specificity or complexation ability. Finding of ionophores with a better pseudoparameters is target of many synthetic chemists in present days.

As it has been reviewed (K. P. Wainwright: Coordination Chemistry Reviews 166 (1997) 35; F. Denat, S. Brandes, R. Guilard: Synlett (2000) 561; S. Liu, D. S. Edwards: Bioconjugate Chemistry 12 (2001) 653) macrocyclic polyaza derivatives are one of the most popular ionophores. These macrocyclic polyaza derivatives have enormous importance e.g. as chelating agents in human radiotherapy or radiodiagnostics with superb pseudoparameters. In future their importance will be strongly progressive. In contrast to this fact, in literature there are very few references describing any preparations of compounds of these structures. To date, there are not described any important and scalable synthetic methods for preparation of macrocyclic polyaza derivatives with one functional group containing phosphorus or arsenic.

Main methodics for the preparation of tricarboxymethylalkylphosphino derivatives, tricarboxymethyl dialkyl phospho derivatives and phosphomethyl derivatives of cyclame and cyclene was described by the firm Therapharm GmbH (Novel chelating agents of tetraazacyclododecane methylphosphonictriacetic acid derivatives and their conjugates, their synthesis and use as diagnostic and therapeutic agents, PCT Int. Appl. (2003), WO 2003008394 A1). However, the patent does not claim any synthetic way of their preparation. Reproduction of the examples stated in this patent is very uneconomical and gives very poor yields, or it can't be reproducted at all. Therefore it is not suitable for preparation of compounds of this type. Monophosphomethyl derivative of cyclame was published by J. Kotek e.a. (Bis(methylphosphonic acid) derivatives of 1,4,8,11-tetraazacyclotetradecane (cyclam). Synthesis, crystal and molecular structures, and solution properties: Collection of Czechoslovak Chemical Communications (2000), 65(8), 1289-1316). Analogous monophosphomethyl derivative of cyclene has been published by Ramachandran Ranganathan (Preparation of 1,4,7,10-tetraazacyclododecanes and multimers as chelating agents with enhanced relaxivity: PCT Int. Appl., WO 9531444 (1995)). Same molecule was claimed by D. A. Sherry and Jeroen Van Westrenen (Preparation of N-substituted-polyazamacrocycles as chelants: U.S. Pat. No. 5,316,757 (1994); Synthesis of polyazamacrocycles with more than one type of side chain chelating groups: WO 9312097 (1993)) and these also has been published (Sulfomethylation of di-, tri-, and polyazamacrocycles: a new route to entry of mixed-side-chain macrocyclic chelates: Bioconjugate Chemistry (1992), 3(6), 524-32). Phosphinic acid esters of cyclene were described by D. Parker e.a. (Synthesis of new macrocyclic aminophosphinic acid complexing agents and their C- and P-functionalized derivatives for protein linkage: Synthesis (1992), (1-2), 63-8) and have been claimed (Tetra-aza macrocycles, processes for their preparation, and their use in magnetic resonance imaging: Eur. Pat. Appl. EP 455380 A2 (1991)).

Preparation of cyclame P,P-bis(hydroxymethyl)phosphinomethyl derivative was described by Kattesh Katti e.a. (Conjugate and method for forming aminomethyl phosphorus conjugates, U.S. Pat. No. 5,948,386 (1999)). The preparation and structure have no importance. Condensed pyrido[a,f]cyclene derivatives has been published by Silvio Aime e.a. ([GdPCP2A(H₂O)₂]⁻: A Paramagnetic Contrast Agent Designed for Improved Applications in Magnetic Resonance Imaging: Journal of Medicinal Chemistry (2000), 43(21), 4017-4024). Interesting are α-alkyl and α-arylderivatives of cyclene monophosphomethyl derivatives described by Xiaodong Li e.a. (Synthesis and NMR Studies of New DOTP-like Lanthanide(III) Complexes Containing a Hydrophobic Substituent on One Phosphonate Side Arm: Inorganic Chemistry (2001), 40 (26), 6572-6579).

Low yields, direct dependence of synthetic method on structure, no scalability, many reaction steps or high cost of carrying out are some of the most important general characteristics of all these described methods.

### DESCRIPTION OF THE INVENTION

The first aspect of the present invention provides micro- to large-scale processes for preparation, manufacturing, production or general synthesis of selective (specific) ligands, chelators, ionophores and complexans on base of polyazamacrocycles of the general formula (1):

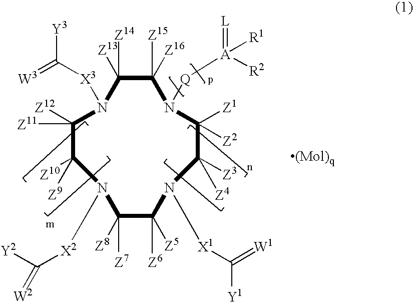

wherein:
A is phosphorus or arsenic;
Z¹⁻¹⁶ is independently selected from a radical of hydrogen; chlorine; bromine; fluorine; iodine; nitro or nitroso; sulpho; or a substituted or unsubstituted straight-chained, branched or cyclic hydrocarbon radical having from 1 to 20 carbon atoms and being saturated or unsaturated with one or more double or triple bonds and optionally containing heteroatoms such as F, Br, Cl, O, N, S and/or P; a substituted or unsubstituted aromatic radical having from 5 up to 18 ring carbon atoms or its aryloxy derivative and including polynuclear aromatic radicals; hydroxyl; alkoxyl; S-substituted or S-unsubstituted thiol; mono- or disubstituted or unsubstituted amine; Z¹⁻¹⁶ also can constitute independently carbonyl and general functional derivatives of carbonyl as oxime, hydrazone, but especially N-substituted or unsubstituted carboimidyl; thiocarbonyl; condensed substituted or unsubstituted benzoderivative; A(L) R¹R²;
n, m is independently 1 or 2;
X¹⁻³ is independently methylene or ethylene substituted as defined for Z¹⁻¹⁶ especially with or without heteroatoms and multiple bonds; carbonyl; N-substituted or unsubstituted carbolmidyl; thiocarbonyl;
Y¹⁻³ is independently methyl substituted as defined for Z¹⁻¹⁶; hydroxyl; O-substituted hydroxyl with Z¹⁻¹⁶; S-substituted thiol; substituted or unsubstituted amine; hydroxylate or thiolate of metal cations or organic cations such as Na, Li, K, Rb, Cs, Ca, Mg, Al, Zn, Mn, Cr, Mo, ⁶⁴Cu, ⁵⁷Cu, ⁶⁷Ga, ⁹⁰Y, ¹¹¹In, ¹⁵³Sm, ¹⁶⁶Ho, ¹⁷⁷Lu, ²⁰¹Tl, ²¹²Bi, ammonium, primary, secondary, tertiary and quarternary alkyl and aryl ammonium, sulphonium and phosphonium salts and their combinations; Y¹⁻³ can constitute independently a substituted hydroxylamine of formula:

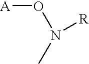

wherein A is independently methyl substituted as defined for Z¹⁻¹⁶; a metal cation or organic cation such as Na, Li, K, Rb, Cs, Ca, Mg, Al, Zn, Mn, Cr, Mo, ⁶⁴Cu, ⁶⁷Cu, ⁶⁷Ga, ⁹⁰Y, ¹¹¹In, ⁵³Sm, ¹⁶⁶Ho, ¹⁷⁷Lu, ²⁰¹Tl, ²¹²Bi, ammonium, primary, secondary, tertiary and quarternary alkyl and aryl ammonium, sulphonium and phosphonium salts and their combinations;
R is independently a radical of hydrogen; substituted or unsubstituted straight-chained, branched or cyclic hydrocarbon radical having from 1 to 20 carbon atoms and being saturated or unsaturated with one or more double or triple bonds and optionally containing heteroatoms such as F, Br, Cl, O, N, S and/or P; a substituted or unsubstituted aromatic radical having from 5 up to 18 ring carbon atoms and including polynuclear aromatic radicals;
Q is independently methylene or ethylene substituted as defined in Z¹⁻¹⁶, ethenylene or ethynylene substituted as defined in Z¹⁻¹⁶; carbonyl; N-substituted or unsubstituted carboimidyl; thiocarbonyl;
p is from 1 to 10;
R¹⁻² is independently hydrogen; halogen; substituted or unsubstituted straight-chained, branched or cyclic hydrocarbon radical having from 1 to 20 carbon atoms and being saturated or unsaturated with one or more double or triple bonds and optionally containing heteroatoms such as F, Br, Cl, O, N, S and/or P; substituted or unsubstituted aromatic radical having from 5 up to 18 ring carbon atoms or its aryloxy derivative and including polynuclear aromatic radicals; hydroxyl; alkoxyl; thiol; thioalcoxyl; substituted or unsubstituted amine; trialkylsilyl; trialkylsilyloxy, triarylsilyl; triarylsilyloxy; hydroxylate or thiolate of metal cations or organic cations such as Na, Li, K, Rb, Cs, Ca, Mg, Al, Zn, Mn, Cr, Mo, ⁶⁴Cu, ⁶⁷Cu, ⁶⁷Ga, ⁹⁰Y, ¹¹¹In, ¹⁵³Sm, ¹⁶⁶Ho, ¹⁷⁷Lu, ²⁰¹Tl, ²¹²Bi, ammonium, primary, secondary, tertiary and quarternary alkyl and aryl ammonium, sulphonium and phosphonium salts and their combinations;
L is oxygen, sulphur, N-substituted or unsubstituted imidyl;
W¹⁻³ is independently oxygen, sulphur, N-substituted or unsubstituted imidyl;
Mol is a protogenic acid, for example, a mineral acid, a substituted or unsubstituted carboxylic, sulphonic, phosphonic and phosphinic acid or a protophilic base, for example, pyridine, tetrahydrofurane, triethylphosphine or a Lewis acid, for example, BF₃, ZnCl₂, AlCl₃, FeBr₃ or a neutral molecule bonded as e.g. in molecular cluster or associate, e.g. chloroform, toluene, water, dioxan, aceton, dimethylformamide cyclodextrine, calix[8]arene, polyethyleneglycole 800;
q is a number from 0 to 10 including a fraction number such as ½ or ⅔ or ¾, 4/3, 3/2.

The “straight-chained, branched or cyclic hydrocarbon radical” according to the present invention particularly relates to C₁-C₁₀ alkyl, C₂-C₁₀ alkenyl, C₂-C₂₀ alkynyl, C₃-C₁₈ cycloalkyl. The C₁₋₁₀ alkyl radicals include methyl, ethyl, propyl, isopropyl, butyl, isobutyl, tertiary butyl, amyl, hexyl, and the like. The C₂₋₁₀ alkenyl radicals include vinyl, propenyl, 1-butenyl, isobutenyl, 2-butenyl, 1-pentenyl, (Z)-2-pentenyl, (E)-2-pentenyl, (Z)-4-methyl-2-pentenyl, (E)-4-methyl-2-pentenyl, pentadienyl, e.g. 1,3- or 2,4-pentadienyl, and the like. Examples of the C₂-C₂₀ alkynyl radicals include such groups as ethynyl, propynyl, 1-butynyl, 2-butynyl, 1-pentynyl, 2-pentynyl, 3-methyl-1-pentynyl, 3-pentynyl, 1-hexynyl, 2-hexynyl, 3-hexynyl and the like. The cycloalkyl groups may be mono-, bi-, tri- or polycyclic and the rings may be fused or bridged. Examples include cyclopropyl, cyclobutyl, cyclopentyl, cyclohexyl, cycloheptyl, cyclooctyl, cyclodecyl, cyclohexenyl, cyclopentenyl, cyclooctenyl, cycloheptenyl, decalynyl decalinyl, hydroindanyl, indanyl, fenchyl, pinenyl, adamantyl, and the like.

The term “aromatic radical” according to the present invention particularly relates to C₅-C₁₀ (hetero)aryl radicals including polynuclear aryl radicals. The heteroaryl radicals contain at least one sulfur, nitrogen or oxygen ring atom, but also may include several of said atoms in the ring. Examples include phenyl, naphthyl, anthracenyl, azulenyl, furyl, thienyl, pyrrolyl, pyrazolyl, pyridinyl, pyrimidinyl, indolyl, quinolyl, acridinyl and the like.

The term “alkoxyl” as used according to the present invention are alkoxyl groups containing from 1 to 6 carbon atoms, especially 1 to 3 carbon atoms, and may be straight-chained or branched. These groups include methoxy, ethoxy, propoxy, butoxy, isobutoxy, tert-butoxy, pentoxy, hexoxy and the like.

The term “substituted” according to the present invention refers to radicals substituted with at least one electron withdrawing and/or at least one electron donating group. Electron withdrawing groups include halo, including bromo, fluoro, chloro, iodo and the like; nitro, carboxy, C₂-C₆ alkenyl, C₂-C₆ alkynyl, formyl, carboxyamido, aryl, quaternary ammonium, haloalkyl such as trifluoromethyl, aryl C₂-C₆ alkanoyl, carbalkoxy and the like. Electron donating groups include such groups as hydroxy, C₂-C₆ alkoxy, including methoxy, ethoxy and the like; C₂-C₆ alkyl, such as methyl, ethyl and the like; amino, C₂-C₆ alkylamino, di(C₂-C₆alkyl)amino, aryloxy such as phenoxy, mercapto, C₂-C₆ alkylthio, C₂-C₆ alkylmercapto, disulfide (C₂-C₆ alkyldithio) and the like. One of ordinary skill in the art will appreciate that some of the aforesaid substituents may be considered to be electron donating or electron withdrawing under different chemical conditions.

The symbol Z¹⁻¹⁶ additionally may be or contain a functional group, particularly a group which is suitable for conjugating the compound of formula I to a binding partner such as a biomolecule. Numerous examples of such coupling groups which e.g. are capable of selectively reacting with amino, thio or hydroxy groups of biomolecules are known in the art. Specific examples of functional groups are alkoxy, Cl, Br, I, NO₂, substituted or unsubstituted amine, carbonyl derivatives, —COOH, NCS, NCO and NHCOCH₂Br, NHCOCH₂I, 2,5-dioxo-2,5-dihydro-pyrrol-1-yl.

In a preferred embodiment of the present invention, R¹⁻² contains a functional group capable of coupling to a binding partner, e.g. a biomolecule. Particularly preferred meanings of R¹⁻² are -θₙ-CH₂)₁₋₄-Ph-Ω or -θₙ-(CH₂)₁₋₄-Ph-Ω or -θₙ-Ph-Ω, wherein θ is O, N, S and Ω is a substituted or unsubstituted amine, —COOH and its esters, preferably esters with derivatives 1-hydroxy-pyrrolidine-2,5-dione, 2-hydroxy-isoindole-1,3-dione, benzotriazol-1-ol, 6-hydroxy-pyrrolo[3,4b]pyridine-5,7-dione, 3-hydroxy-3H-quinazolin-4-one or 6-hydroxy-2H-pyridazin-3-one or preferably esters with phenole derivatives and further more —B(OH)₂, —SH, —OH, —NCS, NCO or —NHCOCH₂Br, —NHCOCH₂I, 2,5-dioxo-2,5-dihydro-pyrrol-1-yl or a carbonyl derivative and n is 0 or 1.

The compounds of the present invention may be complexed with metal ions, preferably with metal ions in the oxidation state+2 or higher. Suitable examples of metal ions are transition metals, lanthanides, actinides, but also main group metal ions. In a preferred embodiment the metal is a radioisotope, e.g. ⁶⁴Cu, ⁶⁷Cu, ⁶⁷Ga, ⁹⁰Y, ¹¹¹In, ¹⁵³Sm, ¹⁶⁶Ho, ¹⁷⁷Lu, 201Tl, ²¹²Bi and combinations thereof. In a further preferred embodiment the metal is Gd.

The compound or the metal complex of the invention may be coupled to a binding partner, particularly a biomolecule such as a peptide, a protein, a glycoprotein, an oligo- or polysaccharide, an oligo- and polyaminosugar or a nucleic acid. Most preferably the biomolecule is an antibody, e.g. a monoclonal antibody, a chimerized antibody, a humanized antibody, a recombinant antibody, e.g. a single chain antibody or an antibody fragment which may be obtained by proteolysis from a complete antibody or by genetic manipulation of antibody-encoding nucleic acids. Methods for preparing suitable antibodies or antibody fragments are known to the skilled person.

The compounds of formula (1) may be synthesized by synthetic routes which are explained in detail below. In the following schematic representation of the synthetic routes of the invention the term describing the residues and substituents of the intermediate and reaction compounds have the same meaning as defined above for formula (1). Newly occurring terms and residues are instead explicitly explained in the description of the following synthetic schemes.

A synthetic route according to the invention comprises the production of the polyazamacrocycles via an intermediate, wherein three of the four nitrogen atoms of the polyazamacrocycle are blocked via a three-functional protecting group (unitriprotected intermediate). The only free nitrogen atom of the unitriprotected intermediate corresponds to the nitrogen atom of the polyazamacrocycle compound of formula (1) on which the phosphorus or arsenic ligand, respectively, will be bonded. Starting out from said unitriprotected intermediate, the intermediate, according to a preferred variant of the process, is reacted with a compound which contains the phosphorus or arsenic ligand, respectively, bound to a leaving group (process variant i). According to another variant of the process, the unitriprotected intermediate is reacted with a compound containing the phosphorus or arsenic ligand, respectively, which contains a reactive double or multiple bond, to which the intermediate can be added (process variant ii). In a still further process variant, first, a reactive compound is added to the free nitrogen atom of the unitriprotected intermediate. In a further reaction step, the phosphorus or arsenic ligand, respectively, is then bound to the resulting group on the free nitrogen (process variant iii).

The above-mentioned process variants i-iii are described in detail below.

i) Synthesis of Compounds of Formula (1) from Unitriprotected Intermediates (N₄GH or N₄G⁻ or N₄H₄(Me)_{w}(X)ᵤ) of Structure:

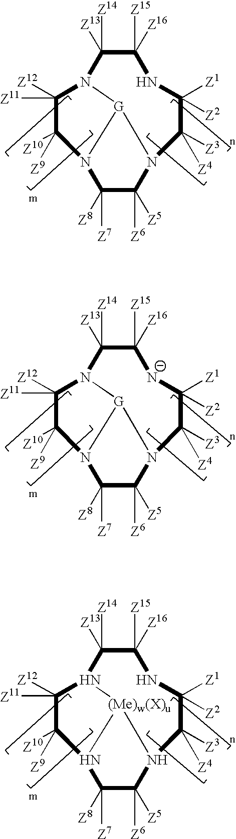

wherein G is CZ¹⁻¹⁶ (include C⁺ as carbocation with Z¹⁻¹⁶ as anion), SiZ¹⁻¹⁶, SnZ¹⁻¹⁶, B, Al, P, As, PO, AsO, PS, AsS, AsZ¹⁻¹⁶, VZ¹⁻¹⁶, PZ¹⁻¹⁶;
Me is metal (or ion), especially: Cu, Ni, Fe, Zn, Cr, Mo, V; X is ligand for example Cl, Br, OH, etc.; u is from 1 to 15; w is 1 or 2 or 3 or ½ or ⅔ or 3/2), or formates TiOZ¹⁻¹⁶, TiNZ¹⁻¹⁶, MoP, MoN;
by condensation with Subst-(Q)ₚA(L)(R¹)(R²) of structure:

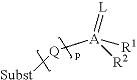

wherein Subst is general leaving group, of structure: —OR, —O⁺RⁱRⁱⁱ, —OSiRⁱRⁱⁱRⁱⁱⁱ, —OCO₂R, —OCONRⁱRⁱⁱ, —OSO₂R, —ON(CORⁱ)(CORⁱⁱ), —NRⁱRⁱⁱ, —(NRⁱRⁱⁱRⁱⁱⁱ))⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, wherein Rⁱ⁻ⁱⁱⁱ are groups of the same type as R. (Subst is for example: hydroxyle, alcoxyl, aryloxyl, alkyl amine, N,N-dialkylamine, N-alkylamine, arylsulfonyloxy, tosyloxy, mesyloxy, triflyloxy, acetoxy, benzoyloxy, N-benztriazolyl, trialkylsilyloxy, hydrazine and N-substituted hydrazine, benzyloxycarbonyloxy, tert.-butyloxycarbonyloxy 1-imidazolyl, succinimidyloxy, N-succinimidyl, N-phthalimidyl, N-phthalimidyloxy, arylthio, thiole, S-alkylthiole etc.), or group which generates in situ or on introduced functional group (Q)ₚA(L)(R¹)(R²) with or without isolation cation or partial positive charge with capability to reaction (photochemically, thermically or electrochemically cleavable groups)
under conditions of general nucleofilic substitution: especially under conditions of phase-transfer catalysis, in aprotic polar solvents or its mixtures (as dimethylformamide or dimethylacetamide or acetonitrile, dimethylsulphoxide or sulpholane or hexamethylphosphortriamide), in micellar medium, in solidphase (for example bonded N₄G⁻ on anex), with or without microwave irradiation, with or without ultrasonic irradiation, under conditions of high pressure (for example in autoclave), in aqueous phase in presence of pH-buffer, in milieu of water-free solvents with or without presence of base (for example: amines, aldimines, carbonates, fluorides, thioethers), enzymatic catalysis, in presence of dehydrating agent or agent reacting with protogenic product reaction or in presence of Lewis acid (e.g. ZnCl₂, BF₃, Et₂O, SiCl₄) etc.
and by next partially or full cleaving of G or (Me)_{w}(X)ᵤ. Both the steps can be solved as one-step reaction or separately.

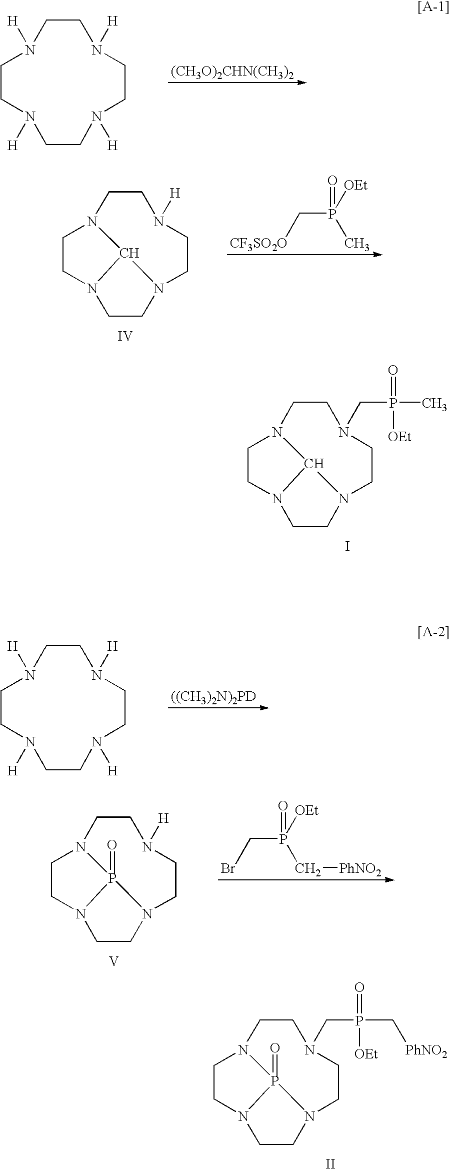

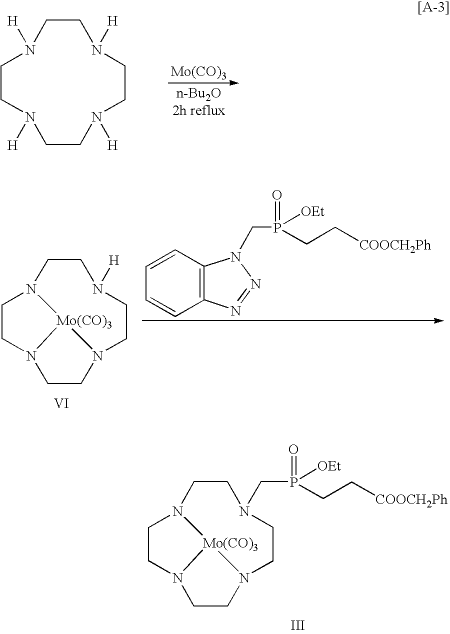

Compounds I-III of the invention can be readily prepared based on schemes A-1-A-3. In all cases are used agents with triprotective functionalities. Next reaction conditions of monosubstitution dependent on whole stability of triprotected intermediates IV-VI in reaction milieu. Nevertheless, there is also direct dependence on reactivity of used alkylating agent. Generally preferred are triprotective groups with high protective ability but also with high cleavage (deprotection) selectivity in mild conditions.

For instance, cyclene necessary as starting crucial reagent is well commercially available. Intermediates IV-VI can be readily synthesized by procedures described in literature (see also D. D. Dischino, E. J. Delaney, J. E. Emswiler, G. T. Gaughan, J. S. Prasad, S. K. Srivastava, M. F. Tweedle: Inorganic Chemistry 30 (1991)1267; Ayoub Filali, Jean-Jacques Yaouanc, Henri Handel Angew. Chem. Int. Ed. Engl. 30 (1991) 560; Véronique Patinec, Jean-Jacques Yaouanc, Jean-Claude Cément, Henri Handel, Hervé des Abbayes, Marek M. Kubicki: Journal Organometallic Chem. 494 (1995) 215). Suitable intermediates I-III for use to prepare compounds of invention may be synthesized by reaction of intermediates IV-VI with dialkylphosphinates derived by active methylene group. Thus e.g. methylenetriflate (scheme A-1), bromomethyl (scheme A-2) and N-1,2,3-benztriazolylmethyl (scheme A-3) derivates can be used. There is obtained the intermediate I by the reaction scheme A-1 under conditions of 24 hours at r.t. (diglyme/presence of DMAP) in 56% yield of separated pure (by HPLC) product I. In the same manner but under conditions of microwave irradiation is obtained product in 69% (reactor: 750 W reflexive or 120 W monomodal; 30-60 s of irradiation by reaction mixture volume). The best results (97%) were obtained in presence of electrochemically-generated calcium in TMED—triglyme system at 5-40° C. with ultrasonic irradiation (40 kHz/120 W/1000 ml of reaction mixture). N-1,2,3-benztriazolyl activation group is generally very suitable for these types of reactions. In these cases reaction conditions are very mild. Thus at 5 hours in acetonitrile reflux intermediate VI affords 92% yield of intermediate III in excellent purity.

ii) Synthesis of Compounds of Formula (1) from Triprotected Intermediates (N₄ GH or N₄G⁻ or N₄H₄(Me)_{w}(X)ᵤ) of the same Structure as in Last Point i)
by addition on (Q)A(L)(R¹)(R²) of structure:

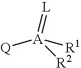

wherein anywhere on Q is double or multiple bond with capability to add intermediates (N₄ GH or N₄G⁻) under conditions of general addition: especially carried out under high-pressure (for example in autoclave), microwave irradiation, under reflux in high boiling solvents, in micellar systems, in solid phase, under cryogenic conditions, under phase transfer catalysis, etc.

Double or multiple bonds can be generated in situ with or without isolation by general elimination methods from (Q-(XY)ₙ)A(L)(R¹)(R²) by elimination of XY, wherein:

XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc.
n is from 1 to 12.
Double or multiple bonds on (Q)A(L)(R¹)(R²) can be generated also from other substituents, which constitute (Q)A(L)(R¹)(R²) under conditions of irradiation (include thermic) or electrochemical reactions, e.g. tetrazenes, cyclic azides, triazenes, dixandiones etc.
   or by addition and subsequent reduction (or in situ reduction) on an intermediate R—C(═W¹⁻³)(Q)ₚₚA(L)(R¹)(R²) of structure:

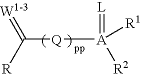

wherein pp is from 0 to 9.

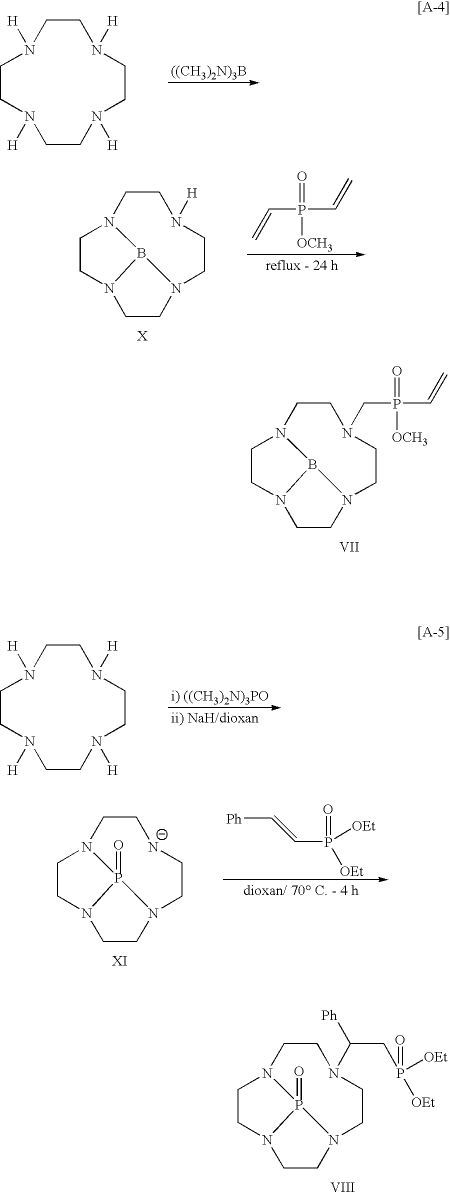

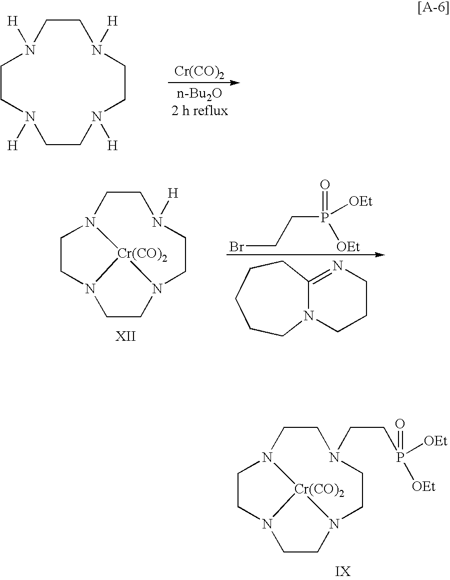

The second manner of preparation is based on addition. Thus, compounds VII-IX of the invention can be readily prepared based on schemes A-4-A-6. In all cases, agents with triprotective functionalities are used. Next reaction conditions of monosubstitution dependent on whole stability of triprotected intermediates X-XII in reaction milieu. However, there is also direct dependence on reactivity of double or multiple bound in structure of alkylating agent. Generally preferred are triprotective groups with high protective ability but also with high cleavage (deprotection) selectivity in mild conditions.

Cyclene necessary as starting reagent is commercially available. Intermediates X-XII can be readily synthesized by procedures described in literature (see also Ayoub Filali, Jean-Jacques Yaouanc, Henri Handel: Angew. Chem. Int. Ed. Engl. 30 (1991) 560; Véronique Patinec, Jean-Jacques Yaouanc, Jean-Claude Clément, Henri Handel, Hervé des Abbayes, Marek M. Kubicki: Journal Organometallic Chem. 494 (1995) 215; Chuburu F., Baccon M. Le., Handel H.: Tetrahedron 57 (2001) 2385). Suitable intermediates VII-IX for use to prepare compounds of invention may be synthesized by reaction of intermediates X-XII with dialkylphosphinates containing double bound. Thus e.g. methyldivinylphosphinate (scheme A-4), diethyl-2-phenylvinylphosphonate (scheme A-5) and in situ generated diethylvinylphosphonate (scheme A-6) can be used. Intermediate VII is obtained by the reaction scheme A-4 under conditions of 24 hours reflux (dry THF-glym 1:1 mixture) in 56% yield. Addition of imide on double bound is represented by scheme A-5. There is obtained 33% of targeted product by stirring in dioxane at 70° C. after four hours. In presence of phase transfer catalyst (15-crown-5) is yield of the same product raised to 59%. Nevertheless, the best yield (72%) was obtained in presence of phase transfer catalyst and after ultrasonic irradiation at 40° C. for 2 hours. The method of in situ generation of reactive agent is very suitable in cases where it is not possible to obtain input derivate with double bounds in appropriate purity. Thus, intermediate IX is obtained in presence of DABCO so called “proton sponge” in 64% yield.

iii) Synthesis of Compounds of Formula (1) from Unitriprotected Intermediates of Structure:

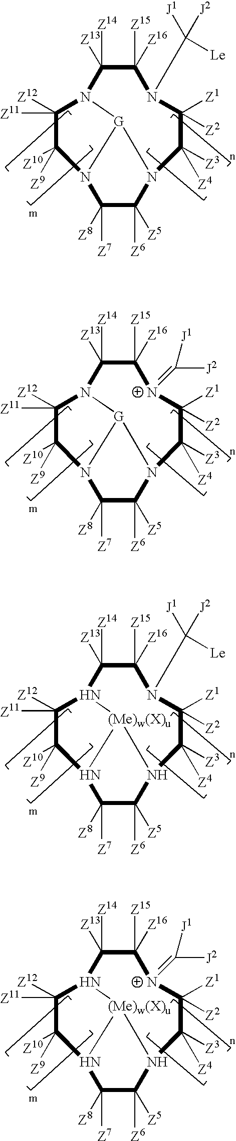

wherein:
G is CZ¹⁻¹⁶ (include C⁺ as carbocation with Z¹⁻¹⁶ as anion), SiZ¹⁻¹⁶, SnZ¹⁻¹⁶, B, Al, P, As, PO, AsO, PS, AsS, AsZ¹⁻¹⁶, VZ¹⁻¹⁶, PZ¹⁻¹⁶;
Me is metal (or ion), especially: Cu, Ni, Fe, Zn, Cr, Mo, V; X is ligand for example Cl, Br, OH, etc.; U is from 1 to 15;
w is 1 or 2 or 3 or ½ or ⅔ or 3/2), or formates TiOZ¹⁻¹⁶, TiNZ¹⁻¹⁶, MoP, MoN;
J¹⁻² is group (substituent, fragment) of the same type as Z¹⁻¹⁶ (J¹⁻² can form substituted methylen) and Le is leaving group, especially of structure: —OR, —OH, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱⁱ)(SO₂Rⁱⁱ), —NSO₂R, -halogene, —NRⁱNRⁱⁱ, —SR, —SO₃H or —SO₂Y¹⁻³, N-benztriazolyl, 1-imidazolyl, succinimidyloxy, N-succinimidyl, N-phthalimidyl, N-phthalimidyloxy wherein Rⁱ⁻ⁱⁱⁱ are groups of the same type as R
by reaction (e.g. condensation) with precursors or their mixture of structure:

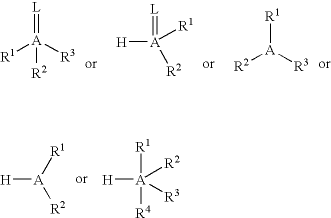

wherein R³ and R⁴ are groups of the same type as R¹⁻². Especially can be used: alkylphosphinic acid, arylphosphinic acid, trialkylphosphites, triarylphosphites, trialkylphosphines, triarylphosphines, dialkylphosphinates, diarylphosphinates, alkylarylphosphinates, dialkylarylphosphites, alkyldiarylphosphites, phosphinic acid, alkylarsenic(III) acid, arylarsenic(III) acid, trialkylarsenic(III), triarylarsenic(III), etc.
under conditions of general nucleofilic substitution: especially under conditions of phase-transfer catalysis, in aprotic polar solvents or its mixtures (as dimethylformamide or dimethylacetamide acetonitrile, dimethylsulphoxide or sulpholane or hexamethylphosphortriamide), in micellar medium, in solidphase (for example bonded N₄G⁻ on anex), with or without microwave irradiation, with or without ultrasonic irradiation, under conditions of high pressure (for example in autoclave), in aqueous phase in presence of pH-buffer, in milieu of water-free solvents with or without presence of base (for example: amines, aldimines, anex, carbonates, fluorides, thioethers), enzymatic catalysis, in presence of dehydrating agent or agent reacting with protogenic product reaction or in presence of Lewis acid as catalysators (for example ZnCl₂, BF₃.Et₂O, SiCl₄) etc.

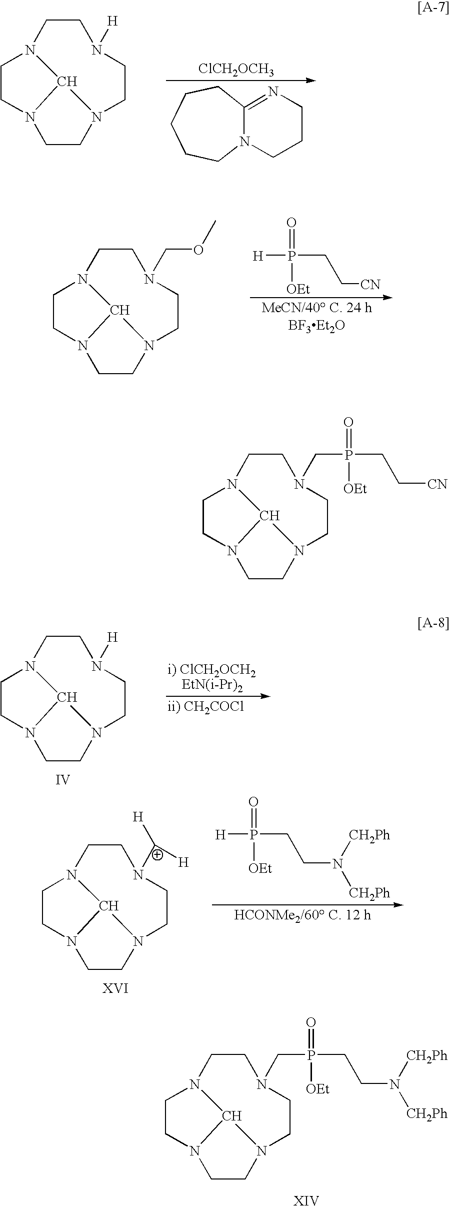

Compounds XIII-XIV of the invention can be prepared based on schemes A-7-A-8. In all cases, intermediates with triprotective functionalities are used. Carbinylated cyclene IV necessary as starting reagent can be readily

synthesized by procedures described in literature. Suitable intermediates XIII-XIV for use to prepare compounds
of invention may be synthesized by reaction of intermediates XV-XVI with alkylphosphinates. Thus e.g. ethylcyanoethylphosphinate (scheme A-7) and ethyl-2-N,N-dibenzylaminoethylphosphinate (scheme A-8) can be used. Cardinal step of the both reaction is methoxymethylation or methenylation respectively. The first intermediate XV can be obtained in high yield by reaction of IV with chlorodimethylether and proton acceptor. In this case there is possible to prepare XV in presence of DABCO so Galled “proton sponge” in 92% yield. The second intermediate XVI is synthesized in similar manner as previous intermediate, but action of reagent as e.g. acetyl chloride is necessary. Overall yield of the reaction is 65%.

Further, the polyazamacrocyclic compounds may be prepared via intermediates, in which the nitrogen atoms are blocked via mono- or bifunctional protective groups. Thereby three of the four nitrogen atoms of the polyazamacrocycle are blocked by mono- or bifunctional independent protective groups, and the resulting protected intermediate (triprotected intermediate) is further reacted with the phosphorus or arsenic ligand, respectively. According to a preferred process variant, the above triprotected intermediate is directly reacted with a compound containing the phosphorus or arsenic ligand, respectively, and a leaving group (process variant iv). In a further process variant, the intermediate is reacted with a compound having a double or multiple bond and containing the phosphorus or arsenic ligand, respectively. The reaction to give the final polyazamacrocycle product is thereby effected in one step (process variant v). The reaction of the above-described triprotected intermediate to give the final polyazamacrocycle derivative product can also be effected by building up the phosphorus or arsenic ligand, respectively, in two steps. Thereby, first, an active methylene or methylidene group is generated on the free nitrogen atom of the intermediate, which is then reacted with a compound containing the phosphorus or arsenic group, respectively, (process variant yl).

The above-mentioned process variants iv-vi are described in detail below.

iv) Synthesis of Compounds of Formula (1) from Triprotected Intermediates (N₄(Prot)₃H/N₄(Prot)₃⁻ or N₄(XC(W(Y))₃H/N₄XC(W(Y))₃⁻) of Structure:

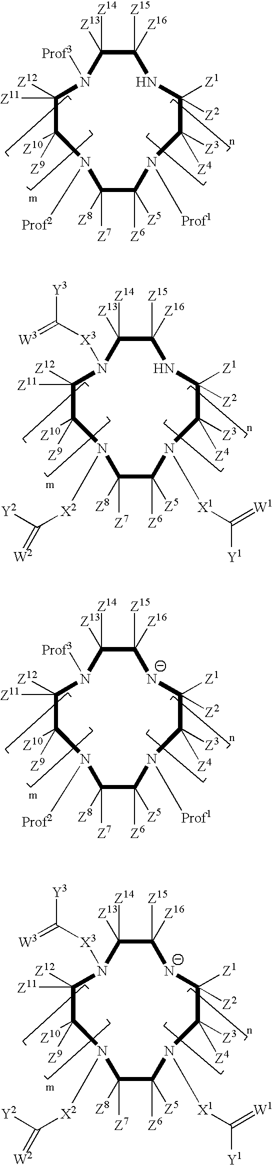

wherein Prot¹⁻³ is independently protective group (or electron pair with negative charge) especially of general structure: —CHO, —COR, —COOR, —CONRⁱRⁱⁱ, —SO₂R, —SR, —R, —SiRⁱRⁱⁱRⁱⁱⁱ, —PORⁱRⁱⁱ, —PSRⁱRⁱⁱ, —PO(ORⁱ)(ORⁱⁱ); protective groups Prot¹ and Prot² or Prot¹ and Prot³ may be also connected to each other especially according to following general structure: —CRⁱRⁱⁱ—, —CO—, —COCO—, —CS—, —C(═NR)—, —COCRⁱRⁱⁱCO—, —CO—R—CO—, [—CRⁱRⁱⁱCRⁱRⁱⁱ]₂SO, [—CRⁱRⁱⁱCRⁱRⁱⁱ]₂SO₂, [—CRⁱRⁱⁱCRⁱRⁱⁱ]₂P(O)OR, [—CRⁱRⁱⁱCRⁱRⁱⁱ]₂NR, —PO(OR)—, —SiRⁱRⁱⁱ—, —SnRⁱRⁱⁱ— wherein Rⁱ⁻ⁱⁱⁱ are groups of the same type as R. Prot¹⁻³ is for example methanesulphonyl, 4-toluenesulphonyl, trifluoromethanesulphonyl, nitrobenzenesulphonyl, benzenesulphonyl, naphthalenesulphonyl, formyl, acetyl, benzoyl, phthaloyl, trifluoroacetyl, tert.-butoxycarbonyl (Boc), 9H-fluoren-9-yl-methoxycarbonyl (Fmoc), benzyloxycarbonyl (Z), ethoxycarbonyl (Eoc), methoxycarbonyl (Meoc), methoxybenzylcarbonyl (Moz), trityl, benzyl, benzhydryl, 4,4-dimethoxytrityl, 4-methoxybenzoyl, ethandioyl, propandloyl, carbonyl, thiocarbonyl etc.
by condensation with Subst-(Q)ₚA(L)(R¹)(R²) of structure:

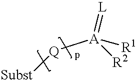

wherein Subst is general leaving group, of structure: —OR, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, wherein Rⁱ⁻ⁱⁱⁱ are groups of the same type as R. (Subst is for example: hydroxyle, alcoxyl, aryloxyl, alkyl amine, N,N-dialkylamine, N-alkylamine, arylsulfonyloxy, tosyloxy, mesyloxy, triflyloxy, acetoxy, benzoyloxy, N-benztriazolyl, trialkylsilyloxy, hydrazine and N-substituted hydrazine, benzyloxycarbonyloxy, tert.-butyloxycarbonyloxy, 1-imidazolyl, succinimidyloxy, N-succinimidyl, N-phthalimidyl, N-phthalimidyloxy, arylthio, thiole, S-alkylthiole etc.), or group which generates in situ or on introduced functional group (Q)ₚA(L)(R¹)(R²) with or without isolation cation or partial positive charge with capability to reaction (photochemically, thermically or electrochemically cleavable groups)
under conditions of general nucleofilic substitution: especially under conditions of phase-transfer catalysis, in aprotic polar solvents or its mixtures (as dimethylformamide or dimethylacetamide acetonitrile, dimethylsulphoxide or sulpholane or hexamethylphosphortriamide), in micellar medium, in solidphase (for example bonded N₄G⁻ on anex), with or without microwave irradiation with or without ultrasonic irradiation, under conditions of high pressure (for example in autoclave), in aqueous phase in presence of pH-buffer, in milieu of water-free solvents with or without presence of base (for example: amines, aldimines, anex, carbonates, fluorides, thioethers), enzymatic catalysis, in presence of dehydrating agent or agent reacting with protogenic product reaction or in presence of Lewis acid as catalysators (for example ZnCl₂, BF₃.Et₂O, SiCl₄) etc.
and by next possible partially or full cleaving of Prot¹⁻³. Both the steps can be solved as one-step reaction or separately.

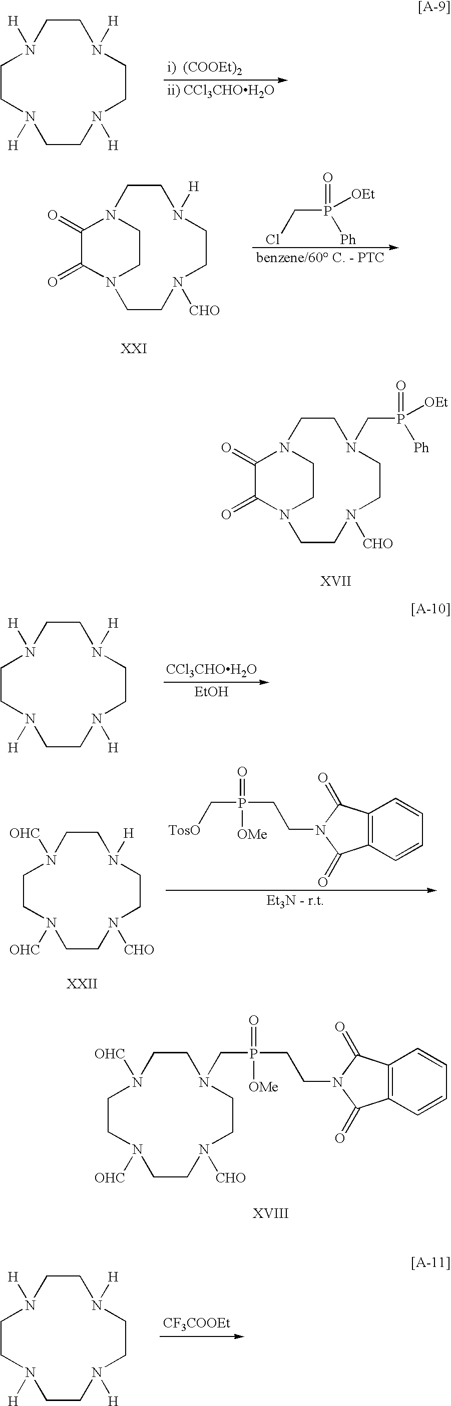

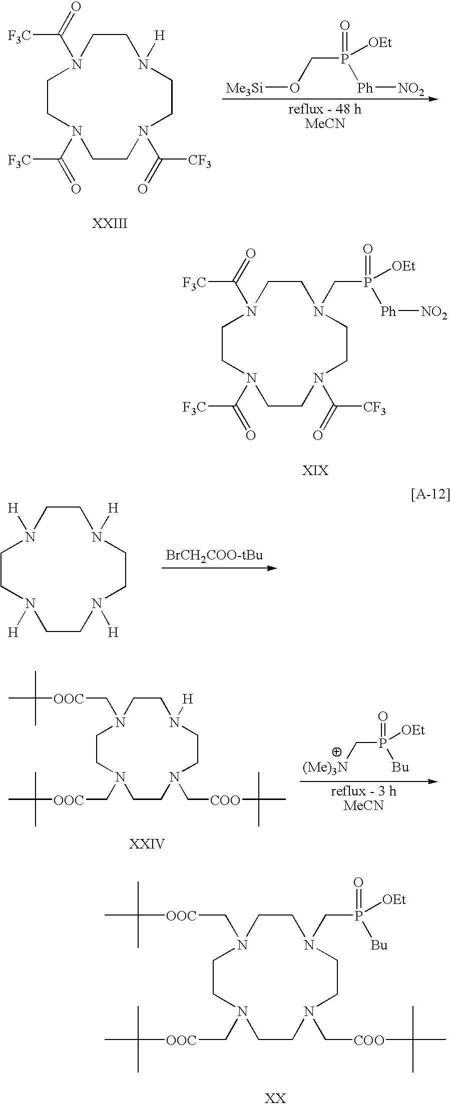

Compounds XVII-XX of the invention can be prepared based on schemes A-9-A-12. In all cases, intermediates with triprotective functionalities are used. As it can be seen on these schemes, there is not necessary to use same protective groups on the each protected nitrogen of cyclene. Reaction conditions at next steps dependent on whole stability of triprotected intermediates XXI-XXIV in reaction milieu. Nevertheless, there is also direct dependence on reactivity of used alkylating agent. Generally preferred are triprotective groups with high protective ability but also with high cleavage (deprotection) selectivity in mild conditions.

Cyclene necessary as starting reagent is very well commercially available. Intermediates XXII-XXIV can be readily synthesized by procedures described in literature. Suitable intermediates XVII-XX for use to prepare compounds of invention may be synthesized by reaction of intermediates XXI-XXIV with dialkylphosphinates containing reactive group bounded on methylene. Thus, e.g. ethylchloromethylphenylphosphinate (scheme A-9), methyl-2-N-phthalimidylethyltosyloxyphosphinate (scheme A-10), ethyl-4-nitrophenyltrimethylsilyloxymethylphosphinate (scheme A-11) and N-(ethylbutylphosphinatomethyl)-N,N,N-trimethylammonium (scheme A-12) can be used.

In all cases, aprotic solvents are preferred. However, there is adequate possibility to obtain products under conditions of phase transfer catalysis in aqueous systems. This alternative is more suitable for large-scale productions. Intermediate XVII is obtained by the reaction scheme A9 under conditions of 48 hours stirring of reaction mixture at 25° C. in 38% yield. If amid is generated by reaction with natrium hydride in ultrasonic bath, there is possible to obtain same product in 59% yield. Use of phase transfer catalysis is documented by scheme A9. However, to suppression of byproducts there is necessary to use phosphinate with high lipofilicity. Suitable catalysator is tetrabutylammonium hydrogensulphate, low yields gives e.g. TEBAC.

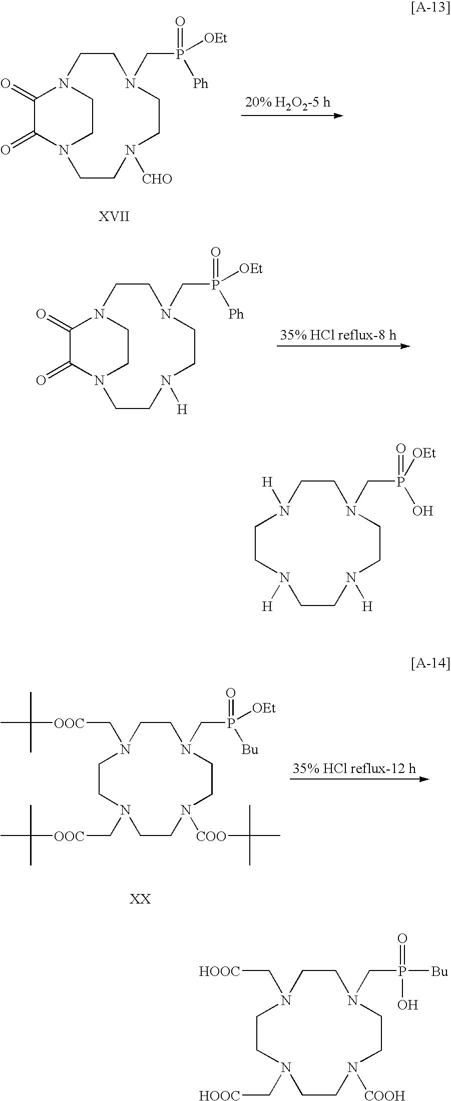

Deprotection of XVII is very simple. Formyl is well cleavage group. There is available selective method for deprotection of formylated amines by action of hydrogen peroxide or generated hydroxyl radicals. Oxalyl, second protective fragment, is cleavaged next by conc. hydrochloride acid by refluxing (scheme A-13). By the same manner is deprotected intermediate XX but there are also cleavaged all tert.-butyls (scheme A-14).

v) Synthesis of Compounds of Formula (1) from Triprotected Intermediates (N₄(Prot)₃H/N₄(Prot)₃⁻ or N₄(XC(W)(Y))₃H/N₄(XC(W)(Y))₃⁻) of the same Structure as in Point iv)
by addition on (Q)A(L)(R¹)(R²) of structure:

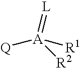

wherein anywhere on Q is double or multiple bond with capability to add intermediates (N₄(Prot)₃H./N₄(Prot)₃, or N₄(XC(W)(Y))₃H./N₄(XC(W)(Y))₃⁻) under conditions of general addition: especially carried out under high-pressure (for example in autoclave), microwave irradiation, under reflux in high boiling solvents, in micellar systems, in solid phase, under cryogenic conditions, under phase transfer catalysis, etc.
Double or multiple bonds can be generated in situ with or without isolation by general elimination methods from (Q-(XY)ₙ)A(L)(R¹)(R²) by elimination of XY, wherein:
   XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc. n is from 1 to 12.
Double or multiple bonds on (Q)A(L)(R¹)(R²) can be generated also from other substituents, which constitute (Q)A(L)(R¹)(R²) under conditions of irradiation (include thermic) or electrochemical reactions, e.g. tetrazenes, cyclic azides, triazenes, dixandiones etc.
   or by addition and subsequent reduction (or in situ reduction) on an intermediate R—C(═W¹⁻³)(Q)ₚₚA(L)(R¹)(R²) of structure:

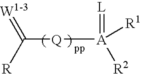

wherein pp is from 0 to 9.

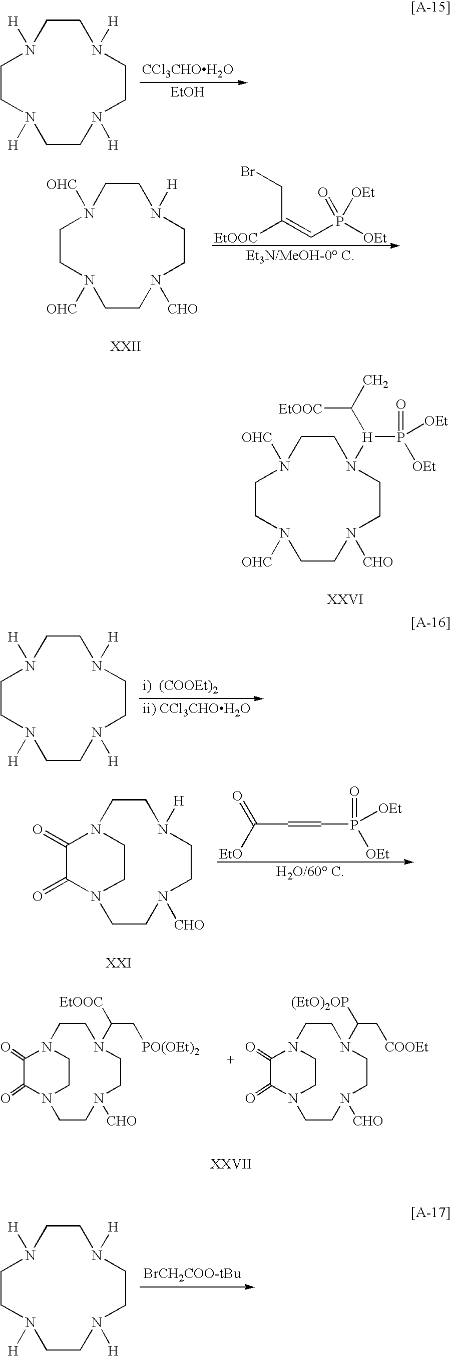

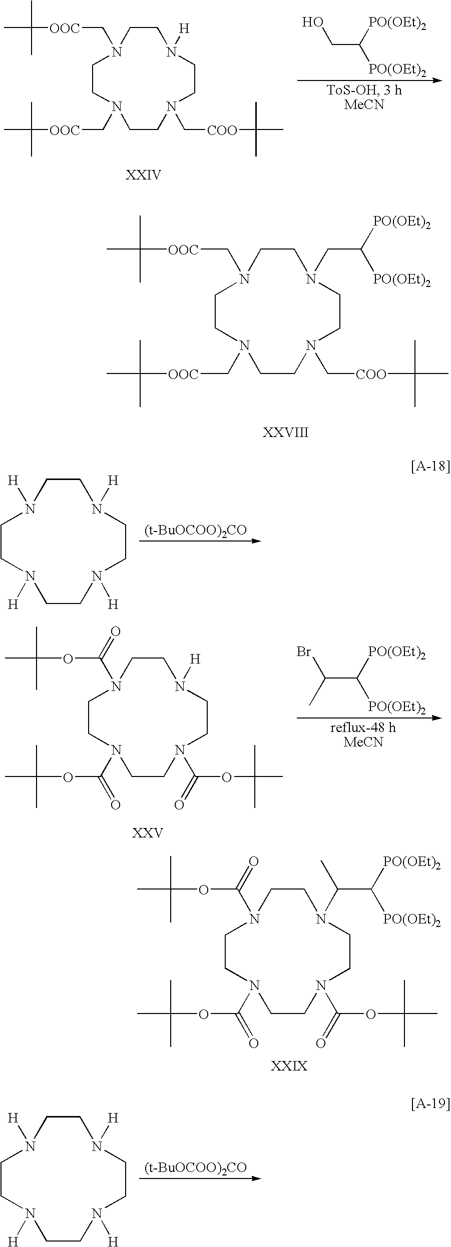

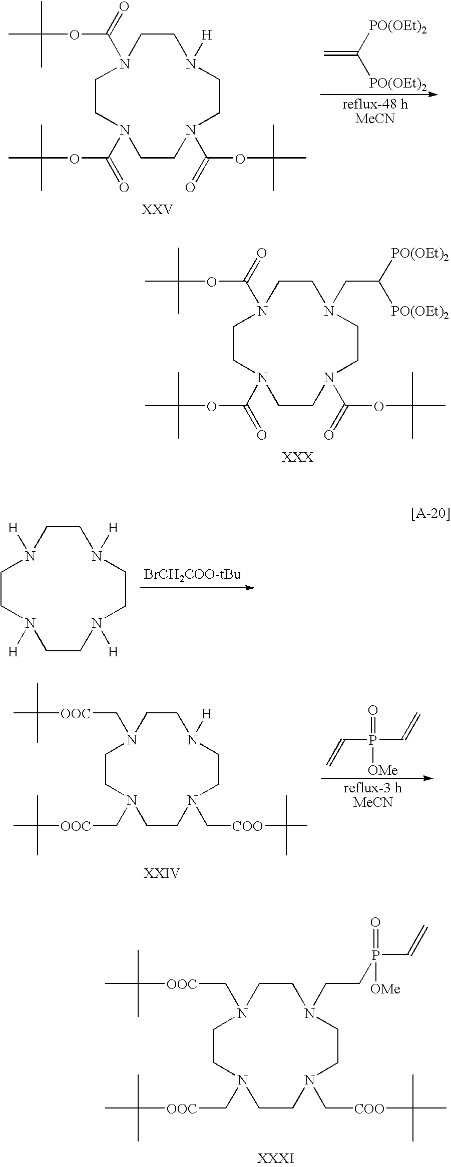

Compounds XXVI-XXXI of the invention can be prepared based on schemes A-15-A-20. In all cases, intermediates with triprotective functionalities are used. There is not necessary to use same protective groups on the each protected nitrogen of cyclene. Reaction conditions at next steps dependent on whole stability of triprotected intermediates XXI-XXII and XXIV-XXV in reaction milieu. Nevertheless, there is also direct dependence on reactivity of used alkylating agent with double bound. Generally preferred are triprotective groups with high protective ability but also with high cleavage (deprotection) selectivity in mild conditions.

Starting reagent—cyclene is commercially available. Intermediates XXI-XXII and XXIV-XXV can be readily synthesized by procedures described in literature. Suitable intermediates XXVI-XXXI for use to prepare compounds of invention may be synthesized by reaction of intermediates XXI-XXII and XXIV-XXV with vinylphosphonates. Reactive vinyl group can be generated in situ by elimination in acidic conditions (scheme A-17) or low basic conditions (scheme A-18). However, if vinylphosphonates (or vinylphosphinates) are available, there are used directly. Thus, e.g. diethyl-3-bromo-2-ethoxycarbonyle-1-propenylphosphonate (scheme A-15), diethyl-2-ethoxycarbonyle-1-propenylphosphonate (scheme A-16), methyldivinylphosphinate (scheme A-20) and tetraethylethylene-1,1-diphosphonate (scheme A-19) can be used. In case of unsymmetrical double bounded reagents, both possible products are obtained (scheme A-16).

(vi) Synthesis of Compounds of Formula (1) from Triprotected Intermediates with Active Methylene or Methylidene Group of Structure:

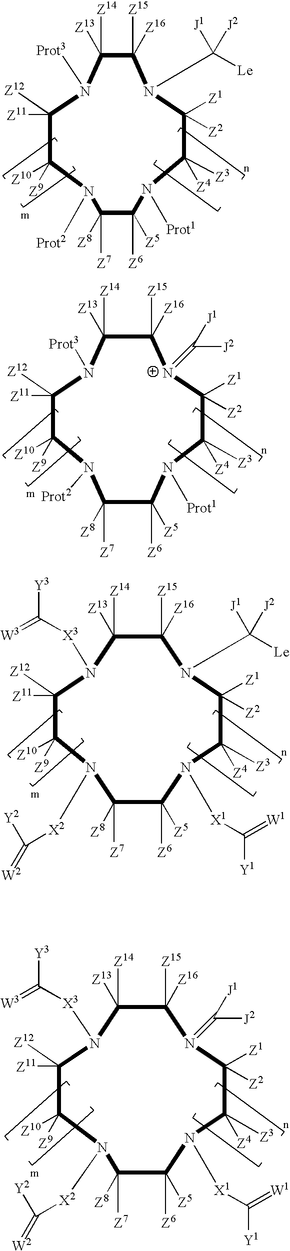

wherein J¹⁻² is group (substituent, fragment) of the same type as Z¹⁻¹⁶ and Le is leaving group, especially of structure: —OR, —OH, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, N-benztriazolyl, 1-imidazolyl, succinimidyloxy, N-succinimidyl, N-phthalimidyl, N-phthalimidyloxy wherein Rⁱ⁻ⁱⁱⁱ are groups of the same type as R
Prot¹⁻³ is independently protective group (or electron pair with negative charge) especially of general structure: —CHO, —COR, —COOR, —CONRⁱRⁱⁱ, —SO₂R, —SR, —R, —SiRⁱRⁱⁱRⁱⁱⁱ, —PORⁱRⁱⁱ, —PSRⁱRⁱⁱ, —PO(OR¹)(ORⁱⁱ); protective groups Prot¹ and Prot² or Prot¹ and Prot³ may be also connected to each other especially according to following general structure: —CRⁱRⁱⁱ—, —CO—, —COCO—, —CS—, —C(═NR)—, —COCRⁱRⁱⁱCO—, —CO—R—CO—, [—CRⁱRⁱⁱCRⁱRⁱⁱ]₂SO, [—CRⁱRⁱⁱCRⁱRⁱⁱ]₂SO₂, [—CRⁱRⁱⁱCRⁱRⁱⁱ]₂P(O)OR, [—CRⁱRⁱⁱCRⁱRⁱⁱ]₂NR, —PO(OR)—, —SiRⁱRⁱⁱ—, SnRⁱRⁱⁱ— wherein Rⁱ⁻ⁱⁱⁱ are groups of the same type as R. Prot¹⁻³ is for example methanesulphonyl, 4-toluenesulphonyl, trifluoromethanesulphonyl, nitrobenzenesulphonyl, benzenesulphonyl, naphthalenesulphonyl, formyl, acetyl, benzoyl, phthaloyl, trifluoroacetyl, tert.-butoxycarbonyl (Boc), 9H-fluoren-9-yl-methoxycarbonyl (Fmoc), benzyloxycarbonyl (Z), ethoxycarbonyl (Eoc), methoxycarbonyl (Meoc), methoxybenzylcarbonyl (Moz), trityl, benzyl, benzhydryl, 4,4′-dimethoxytrityl, 4-methoxybenzoyl, ethandioyl, propandioyl, carbonyl, thiocarbonyl etc.
by reaction (e.g. condensation) with precursors or their mixture of structure:

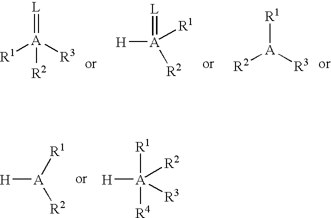

wherein R³ and R⁴ are groups of the same type as R¹⁻². Especially can be used: alkylphosphinic acid, arylphosphinic acid, trialkylphosphites, triarylphosphites, trialkylphosphines, triarylphosphines, dialkylphosphinates, diarylphosphinates, alkylarylphosphinates, dialkylarylphosphites, alkyldiarylphosphites, phosphinic acid, alkylarsenic(III) acid, arylarsenic(III) acid, trialkylarsenic(III), triarylarsenic(III), etc.
under conditions of general nucleofilic substitution: especially under conditions of phase-transfer catalysis, in aprotic polar solvents or its mixtures (as dimethylformamide or dimethylacetamide acetonitrile, dimethylsulphoxide or sulpholane or hexamethylphosphortriamide), in micellar medium, in solidphase (for example bonded N₄G⁻ on anex), with or without microwave irradiation, with or without ultrasonic irradiation, under conditions of high pressure (for example in autoclave), in aqueous phase in presence of pH-buffer, in milieu of water-free solvents with or without presence of base (for example: amines, aldimines, anex, carbonates, fluorides, thioethers), enzymatic catalysis, in presence of dehydrating agent or agent reacting with protogenic product reaction or in presence of Lewis acid as catalysators (for example ZnCl₂, BF₃.Et₂O, SiCl₄) etc.

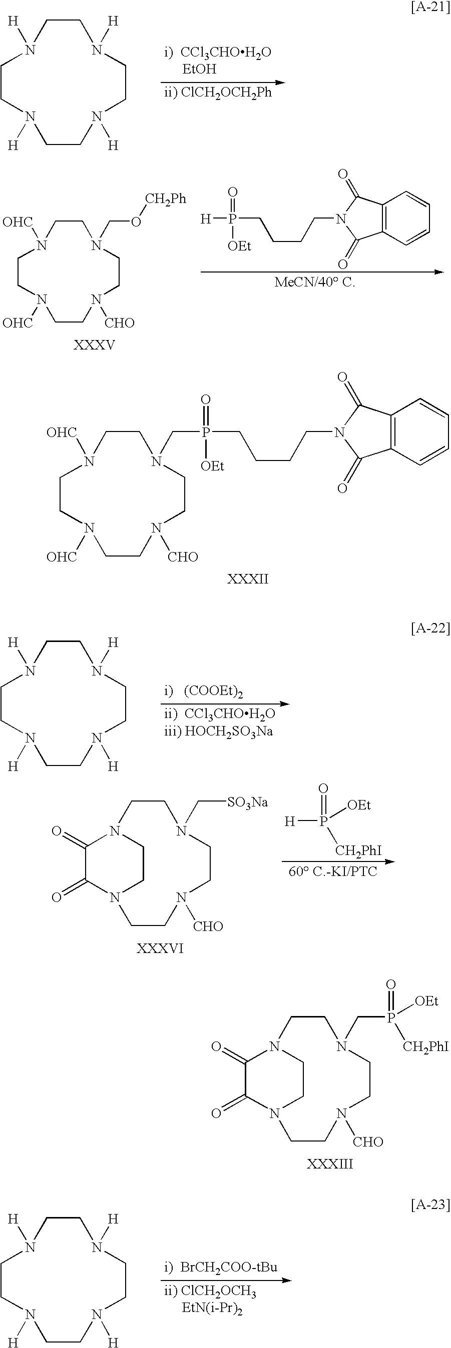

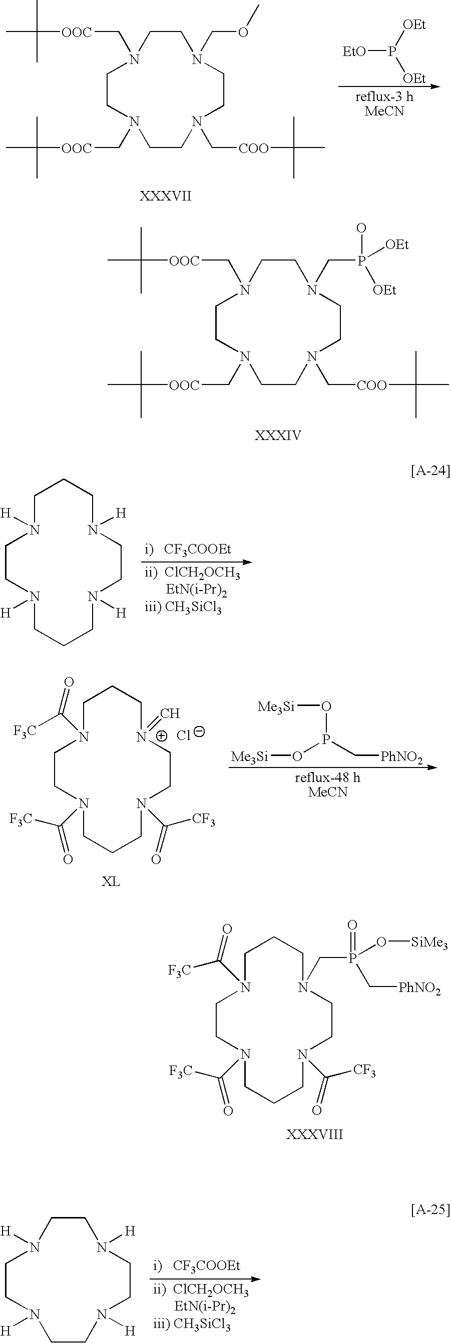

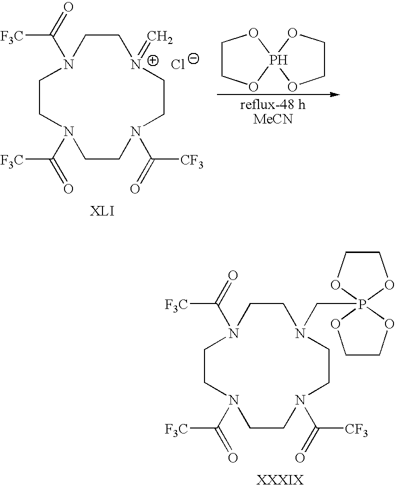

Compounds XXXII-XXXIX of the invention can be prepared based on schemes A-21-A25. In all cases, intermediates with triprotective functionalities are used. It is not necessary to use the same protective groups on each protected nitrogen of cyclene. At next steps reaction conditions depend on whole stability of triprotected intermediates XXXV-XLI in reaction milieu. Nevertheless, there is also direct dependence on reactivity of used alkylating agent. Generally preferred are triprotective groups with high protective ability, but also with high cleavage (deprotection) selectivity in mild conditions.

Cyclene (as well as cyclame) necessary as starting reagent is very well commercially available. Suitable intermediates XXXII-XXXIX for use to prepare compounds of invention may be synthesized by reaction of intermediates XXXV-XLI with alkylphosphinates, trialkylphosphites or arylphosphanes. Thus, e.g. ethyl 4-N-phthalimidylethyltosyloxyphosphinate (scheme A-21), ethyl 4-iodobenzylphosphinate (scheme A-22), triethylphosphite (scheme A-23) and P,P-bis(trimethylsilyloxy)-4-nitrophenylphosphane (scheme A-24) can be used. There is strong dependence of reaction conditions on reactivity of cyclene (as well as cyclame) intermediate XXXV-XLI.

In all cases, aprotic solvents are preferred. Intermediate XXXII is obtained by the reaction scheme A-21 under conditions of 48 hours stirring of reaction mixture in acetonitrile at 40° C. in 51% yield. In same manner is available intermediate XXXIV by reaction of XVII with triethylphosphite in acetonitrile under reflux temperature.

Due to high reactivity of methylsulphonates, yields of method on scheme A-22 are usually excellent. Presence of alkali iodides is not necessary, but in the most cases positively increased yield. If there are some reasons, the reaction can be carried out under conditions of phase-transfer catalysis. This alternative is very suitable for large scale operations.

A further synthesis scheme according to the present invention provides that the production of the polyazamacrocycle derivatives of formula (1) starts out from unprotected intermediates. One process variant according to the invention starting out from unprotected intermediates thereby involves reaction of said intermediate with a compound containing the phosphorus or arsenic ligand, respectively, as well as a leaving group (process variant vii). Further, starting out from the unprotected intermediate, the polyazamacrocycle derivative can be obtained by reacting the unprotected intermediate with a compound containing the phosphorus or arsenic ligand, respectively, as well as a reactive double or multiple bond for addition to the nitrogen atom (process variant viii). According to this synthetic scheme a mixture of mono-, bi-, try and tetrasubstituted polyazamacrocycles is obtained. Preferably a separation of the monosubstituted derivatives is carried out, which is then further reacted to build up the —X¹⁻³—C(═W¹⁻³)Y¹⁻³ ligand on the other three nitrogen atoms, thereby obtaining the polyazamacrocycle derivatives of formula (1).

The above-mentioned process variants vii and viii are described in detail below.

vii) Synthesis of Compounds of Formula (1) from Unprotected Ligands N₄H₄/N₄H₃⁻ of Structure:

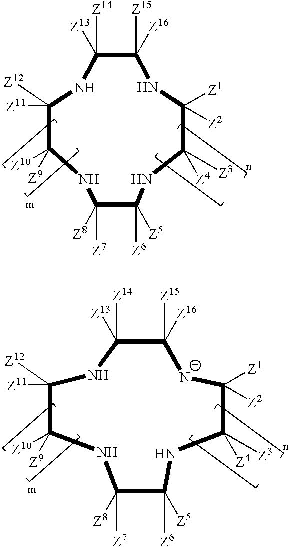

by condensation with Subst-(Q)ₚA(L)(R¹)(R²) of structure:

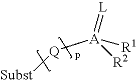

wherein Subst is general leaving group, of structure: —OR, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or SO₂Y¹⁻³, wherein Rⁱ⁻ⁱⁱⁱ are groups of the same type as R. (Subst is for example: hydroxyle, alcoxyl, aryloxyl, alkyl amine, N,N-dialkylamine, N-alkylamine, arylsulfonyloxy, tosyloxy, mesyloxy, triflyloxy, acetoxy, benzoyloxy, N-benztriazolyl, trialkylsilyloxy, hydrazine and N-substituted hydrazine, benzyloxycarbonyloxy, tert.-butyloxycarbonyloxy, 1-imidazolyl, succinimidyloxy, N-succinimidyl, N-phthalimidyl, N-phthalimidyloxy, arylthio, thiole, S-alkylthiole etc.), or group which generates in situ or on introduced functional group (Q)ₚA(L)(R¹)(R²) with or without isolation cation or partial positive charge with capability to reaction (photochemically thermically or electrochemically cleavable groups)
under conditions of general nucleofilic substitution: especially under conditions of high dilution, under conditions of phase-transfer catalysis, in aprotic polar solvents or its mixtures (as dimethylformamide or dimethylacetamide acetonitrile, dimethylsulphoxide or sulpholane or hexamethylphosphortriamide), in micellar medium, in solidphase (for example bonded N₄G⁻ on anex), with or without microwave irradiation, with or without ultrasonic irradiation, under conditions of high pressure (for example in autoclave), in aqueous phase in presence of pH-buffer, under conditions of N-alkylation in presence cations of metals (e.g. calcium, magnesium, cooper, nickel, iron, lithium) or organic cations (e.g. tetramethylammonium), in millieu of water-free solvents with or without presence of base (for example: amines, aldimines, carbonates, fluorides, thioethers), in general two or multiphase systems etc.
with or without separation of monosubstituted ligand N₄H₃(Q)ₚA(L)(R¹)(R²) from reaction mixture. Separation can be carried out by chromatography techniques as: HPLC or LC, ionex chromatography or on ionex column generally, preparative TLC, paper chromatography, gel chromatography, etc., especially by gradient elution on HPLC or LC, or by extraction from water solutions to water immiscible solvents or its mixtures (e.g. dichloromethane, chloroform, ethyl acetate, 1-butylacetate, chlorobenzene, hexane) continuously or discontinuously, at high temperatures or under cooling (e.g. by cryogenic techniques). Separation also can be carried out by precipitation or coagulation, by freezing out, by sublimation out of reactant, by continuous extracting out monosubstituted ligand N₄H₃(Q)ₚA(L)(R¹)(R²) or by-products, etc.
   and possible next reaction
   a) with 3 moles of reactant Subst-(X^{t})C(Y^{t})(W^{t}) (t is 1 or 2 or 3) or independently by step reactions with Subst-(X¹)C(Y¹)(W¹), Subst-(X²)C(Y²)(W²) and Subst-(X²)C(Y²)(W²)) of structure:

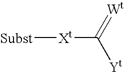

wherein Subst is general leaving group, of structure: —OR, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, wherein Rⁱ⁻ⁱⁱⁱ are groups of the same type as R. (Subst is for example: hydroxyle, alcoxyl, aryloxyl, alkyl amine, N,N-dialkylamine, N-alkylamine, arylsulfonyloxy, tosyloxy, mesyloxy, triflyloxy, acetoxy, benzoyloxy, N-benztriazolyl, trialkylsilyloxy, hydrazine and N-substituted hydrazine, benzyloxycarbonyloxy, tert.-butyloxycarbonyloxy, 1-imidazolyl, succinimidyloxy, N-succinimidyl, N-phthalimidyl, N-phthalimidyloxy, arylthio, thiole, S-alkylthiole etc.), or group which generates in situ or on introduced functional group (X^{t})C(Y^{t})(W^{t}) with or without isolation cation or partial positive charge with capability to reaction (photochemically, thermically or electrochemically cleavable groups)
or by reaction with an intermediate Subst-(CZ¹Z²)ₙCN or by reaction with an intermediate generated in situ by general reaction of HCN and Z¹-C(=L)-Z² and by next hydrolysis with or without isolation of structure:

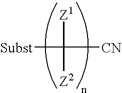

wherein n is 1 or 2,
or by reaction with intermediates Subst-(CZ¹Z²)ₙC(W^{t-t″}Rⁱ⁻ⁱⁱⁱ)₃ or Subst-(CZ¹Z²)ₙC(W^{t-t′}Rⁱ⁻ⁱⁱ)₂R or Subst-(CZ¹Z²)ₙC(═W¹⁻³)R or —(CZ¹Z²)(CZ³Z⁴)W¹⁻³— and by next hydrolytic, oxidative, reduction cleavage of structure:

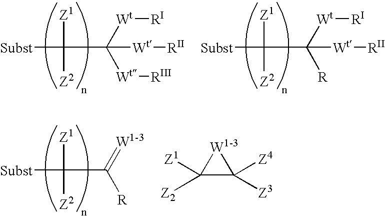

wherein Rⁱ⁻ⁱⁱⁱ is independently group of the same type as R¹⁻², t, t′ and t″ is 1 or 2 or 3, n is 1 or 2.
b) by addition on an intermediate Q-C(Y^{t})(W^{t}) (t is 1 or 2 or 3) or independently by step-reaction with Q-C(Y¹)(W¹), Q-C(Y²)(W²) and Q-C(Y³)(W³) of structure:

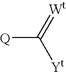

wherein anywhere on Q is double or multiple bond with capability to add intermediates under conditions of general addition: especially carried out under high-pressure (for example in autoclave), microwave irradiation, under reflux in high boiling solvents, in micellar systems, in solid phase, under cryogenic conditions, under phase transfer catalysis, etc.
Double or multiple bonds can be generated in situ with or without isolation by general elimination methods from (Q-(XY)ₙ)—(X^{t})C(Y^{t})(W^{t}) by elimination of XY, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc.
   n is from 1 to 4.
Double or multiple bonds on (Q)-(X^{t})C(Y^{t})(W^{t}) can be generated also from other substituents, which constitute (Q)-C(X^{t})C(Y^{t})(W^{t}) under conditions of irradiation (include thermic) or electrochemical reactions, e.g. tetrazenes, cyclic azides, triazenes, dixandiones etc.
or by addition with an intermediate Q-CN and by next hydrolysis with or without isolation of structure:

Q-CN
Double or multiple bonds can be generated in situ with or without isolation by general elimination of XY from (Q-(XY)ₙ)—CN, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc.
   n is from 1 to 4.
Double or multiple bonds on Q-CN can be generated also from other substituents, which constitute Q-CN under conditions of irradiation (include thermic) or electrochemical reactions, e.g. tetrazenes, cyclic azides, triazenes, dixandiones etc.
or by addition on intermediates Q-C(W^{t-t″}Rⁱ⁻ⁱⁱⁱ)₃ or Q-C(W^{t-t′}Rⁱ⁻ⁱⁱ)₂R or Q-C(═W¹⁻³)R and by next hydrolytic, oxidative, reduction cleavage of structure:

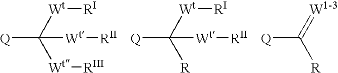

wherein Rⁱ⁻ⁱⁱⁱ is independently group of the same type as R¹⁻², t, t′ and t″ is 1 or 2 or 3.
Double or multiple bonds can be generated in situ with or without isolation by general elimination of XY from (Q-(XY)ₙ)—C(W^{t-t″}Rⁱ⁻ⁱⁱⁱ)₃ or (Q-(XY)ₙ)—C(W^{t-t′}Rⁱ⁻ⁱⁱ)₂R or (Q-(XY)ₙ)-C(═W¹⁻³)R, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarbolylates, hydrogen or metal sulphonate or substituted sulphonate, etc.
n is from 1 to 4.
Double or multiple bonds on Q-C(W^{t-t″}Rⁱ⁻ⁱⁱⁱ)₃ or Q-C(W^{t-t′}Rⁱ⁻ⁱⁱ)₂R or Q-C(═W¹⁻³)R can be generated also from other substituents, which constitute Q-C(W^{t-t″}Rⁱ⁻ⁱⁱⁱ)₃ or Q-C(W^{t-t′}Rⁱ⁻ⁱⁱ)₂R or Q-C(═W¹⁻³)R under conditions of irradiation (include thermic) or electrochemical reactions, e.g. tetrazenes, cyclic azides, triazenes, dixandiones etc.
   c) or by addition and subsequent reduction on an intermediate R^{t}—C(═W^{t′})(CZ¹Z²)_{f}C(Y^{t})(W^{t}) or independently by step-reaction and subsequent reduction or in situ reduction on R—C(═W^{t′})(CZ¹Z²)_{f}C(Y¹)(W¹), R—C(═W^{t′})(CZ³Z⁴)_{f}C(Y²)(W²) and R—C(═W^{t′})(CZ⁵Z⁶)_{f}C(Y³)(W³) of structure:

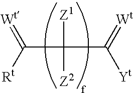

wherein R^{t} is independently group of the same type as R¹⁻², f is 0 or 1, t and t′ is 1 or 2 or 3.
or by addition on intermediates R^{t}—C(═W^{t′})(CZ¹Z²)_{f}C(W^{t-t″}Rⁱ⁻ⁱⁱⁱ)₃ or R^{t}—C(═W^{t′})(CZ¹Z²)_{f}C(W^{t-t′}Rⁱ⁻ⁱⁱ)₂R or R; —C(═W^{t′})(CZ¹Z²)_{f}C(═W¹⁻³)R and by subsequent reduction or in situ reduction or by subsequent hydrolytic, oxidative, reduction cleavage of structure:

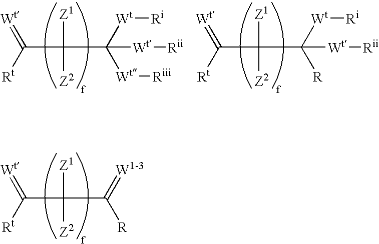

wherein R^{t} is independently group of the same type as R¹⁻², Rⁱ⁻ⁱⁱⁱ is independently group of the same type as R¹⁻², t, t′ and t″ is 1 or 2 or 3, f is 0 or 1.

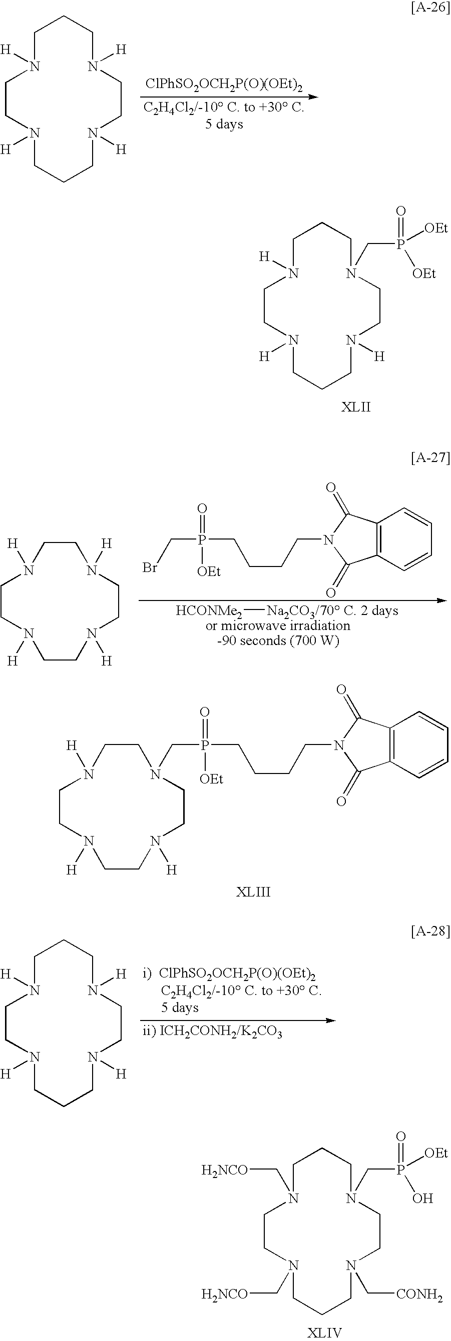

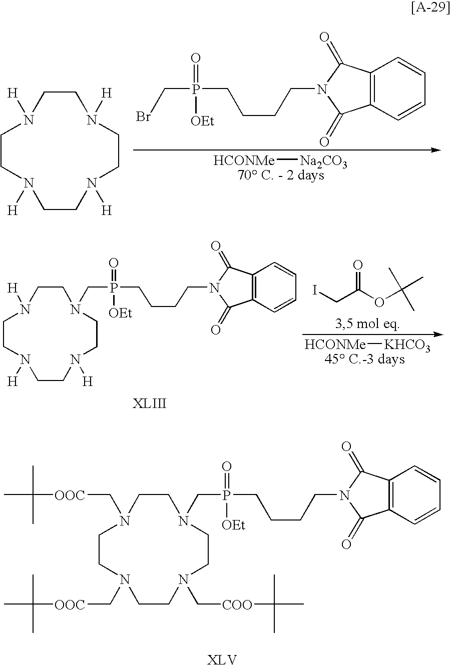

Compounds XLII-XLV of the invention can be prepared based on schemes A-26-A 29. In all cases, intermediates with uniprotective functionalities are used or uniprotective derivatives are targeted compounds of the invention directly.

Either cyclene or cyclame are commercially available. Process of selective monosubstitution is subject of the invention. Generally can be alkylations carried out by procedures described in literature. Suitable intermediates XLII-XLV may be synthesized by reaction of cyclene or cyclame with alkylating reagents. Thus, e.g. ethyl 4-N-phthalimidylbutylbromomethylphosphinate (scheme A-27) can be used in case of cyclene. Diethyl 4-chlorobenzenesulphonyloxyphosphonate can be used in case of cyclame (scheme A-28). The first step—the monoalkylation—can be executed under conditions of reaction in aprotic solvents. Nevertheless, if there are some reasons, the reaction can be carried out under conditions of phase-transfer catalysis. This alternative is very suitable for large-scale operations. Reaction on scheme A-26 needs specific conditions.

Tralkylations, by schemes A-28 and A-29, can be carried out by large number of methods of alkylations on secondary amines. In case of tert.-butoxycarbonylmethylation of XLIII tert.-butyl iodoacetate gives almost quantitative yield of tri-tert.-butoxycarbonylmethylated product in very mild conditions (45° C., 3 days). Solvent-base system composition is cardinal aspect of this method. System cesium carbonate-dry N-methylpyrrolidone gives quantitative yields. Almost quantitative yields give systems: potassium hydrogencarbonate—dry dimethylformamide; potassium carbonate-dry dimethylacetamide; potassium carbonate —N-methylpyrrolidone. Acetamidation by scheme A-28 proceeds in the same manner.

viii) Synthesis of Compounds of Formula (1) from Unprotected Ligands N₄H₄/N₄H₃⁻ of Structure:

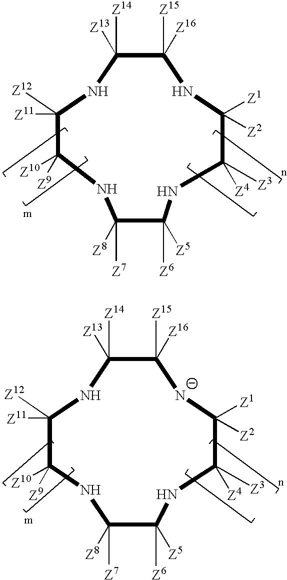

by addition on (Q)A(L)(R¹)(R²) of structure:

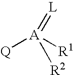

wherein anywhere on Q is double or multiple bond with capability to add intermediates (N₄GH or N₄G⁻) under conditions of general addition: especially carried out under high-pressure (for example in autoclave), microwave irradiation, under reflux in high boiling solvents, in micellar systems, in solid phase, under cryogenic conditions, under phase transfer catalysis, etc.
Double or multiple bonds can be generated in situ with or without isolation by general elimination methods from (Q-(XY)ₙ)A(L)(R¹)(R²) by elimination of XY, wherein:
   XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc.

### n is From 1 to 12.

Double or multiple bonds on (Q)A(L)(R¹)(R²) can be generated also from other substituents, which constitute (Q)A(L)(R¹)(R²) under conditions of irradiation (include thermic) or electrochemical reactions, e.g. tetrazenes, cyclic azides, triazenes, dixandiones etc.
with or without separation of monosubstituted ligand N₄H₄ from reaction mixture. Separation can be carried out by chromatography techniques as: HPLC or LC, ionex chromatography or on ionex column generally, preparative TLC, paper chromatography, gel chromatography, etc., especially by gradient elution on HPLC or LC, or by extraction from water solutions to water immiscible solvents or its mixtures (e.g. dichloromethane, chloroform, ethyl acetate, 1-butylacetate, chlorobenzene, hexane) continuously or discontinuously, at high temperatures or under cooling (e.g. by cryogenic techniques). Separation also can be carried out by precipitation or coagulation, by freezing out, by sublimation out of reactant etc.
   or by addition and subsequent reduction (or in situ reduction) on an intermediate R—C(═W¹⁻³)(Q)ₚₚA(L)(R¹)(R²) of structure:

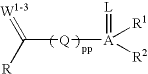

wherein pp is from 0 to 9.
and possible next reaction
   a) with 3 moles of reactant Subst-(Q)C(Y^{t})(W^{t}) (t is 1 or 2 or 3) or independently by step reactions with Subst-(X¹)C(Y¹)(W¹), Subst-(X²)C(Y²)(W²) and Subst-(X²)C(Y²)(W²)) of structure:

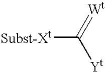

wherein Subst is general leaving group, of structure: —OR, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, wherein Rⁱ⁻ⁱⁱⁱ are groups of the same type as R. (Subst is for example: hydroxyle, alcoxyl, aryloxyl, alkyl amine, N,N-dialkylamine, N-alkylamine, arylsulfonyloxy, tosyloxy, mesyloxy, triflyloxy, acetoxy, benzoyloxy, N-benztriazolyl, trialkylsilyloxy, hydrazine and N-substituted hydrazine, benzyloxycarbonyloxy, tert.-butyloxycarbonyloxy, 1-imidazolyl, succinimidyloxy, N-succinimidyl, N-phthalimidyl, N-phthalimidyloxy, arylthio, thiole, S-alkylthiole etc.), or group which generates in situ or on introduced functional group (X^{t})C(Y^{t})(W^{t}) with or without isolation cation or partial positive charge with capability to reaction (photochemically, thermically or electrochemically cleavable groups)
or by reaction with an intermediate Subst-(CZ¹Z²)ₙCN or by reaction with an intermediate generated in situ by general reaction of HCN and Z¹-C(=L)-Z² and by next hydrolysis with or without isolation of structure:

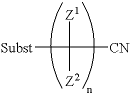

wherein n is 1 or 2,
or by reaction with intermediates Subst-(CZ¹Z²)ₙC(W^{t-t″}Rⁱ⁻ⁱⁱⁱ)₃ or Subst-(CZ¹Z²)ₙC(W^{t-t′}Rⁱ⁻ⁱⁱ)₂R or Subst-(CZ¹Z²)ₙC(═Y¹⁻³)R or —(CZ¹Z²)(CZ³Z⁴)W¹⁻³— and by next hydrolytic, oxidative, reduction cleavage of structure:

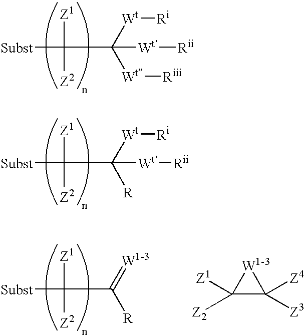

wherein Rⁱ⁻ⁱⁱⁱ is independently group of the same type as R¹⁻², t, t′ and t″ is 1 or 2 or 3, n is 1 or 2.
b) by addition on an intermediate Q-C(Y^{t})(W^{t}) (t is 1 or 2 or 3) or independently by step-reaction with Q-C(Y¹)(W¹), Q-C(Y²)(W²) and Q-C(Y³)(W³) of structure:

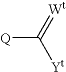

wherein anywhere on Q is double or multiple bond with capability to add intermediates under conditions of general addition: especially carried out under high-pressure (for example in autoclave), microwave irradiation, under reflux in high boiling solvents, in micellar systems, in solid phase, under cryogenic conditions, under phase transfer catalysis, etc.
Double or multiple bonds can be generated in situ with or without isolation by general elimination methods from (Q-(XY)ₙ)—(X^{t})C(Y^{t})(W^{t}) by elimination of XY, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc.
n is from 1 to 4.
Double or multiple bonds on (Q)-(X^{t})C(Y^{t})(W^{t}) can be generated also from other substituents, which constitute (Q)-(X^{t})C(Y^{t})(W^{t}) under conditions of irradiation (include thermic) or electrochemical reactions, e.g. tetrazenes, cyclic azides, triazenes, dixandiones etc.
or by addition with an intermediate Q-CN and by next hydrolysis with or without isolation of structure:

Q-CN
Double or multiple bonds can be generated in situ with or without isolation by general elimination of XY from (Q-(XY)ₙ)—CN, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc.
n is from 1 to 4.
Double or multiple bonds on Q-CN can be generated also from other substituents, which constitute Q-CN under conditions of irradiation (include thermic) or electrochemical reactions, e.g. tetrazenes, cyclic azides, triazenes, dixandiones etc.
or by addition on intermediates Q-C(W^{t-t″}Rⁱ⁻ⁱⁱⁱ)₃ or Q-C(W^{t-t′}Rⁱ⁻ⁱⁱ)₂R or Q-C(═W¹⁻³)R and by next hydrolytic, oxidative, reduction cleavage of structure:

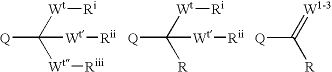

wherein Rⁱ⁻ⁱⁱⁱ is independently group of the same type as R¹⁻², t, t′ and t″ is 1 or 2 or 3.
Double or multiple bonds can be generated in situ with or without isolation by general elimination of XY from (Q-(XY)ₙ)—C(W^{t-t″}Rⁱ⁻ⁱⁱⁱ)₃ or (Q-(XY)ₙ)—C(W^{t-t′}Rⁱ⁻ⁱⁱⁱ)₂R or (Q-(XY)ₙ)—C(═W¹⁻³)R, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc.
n is from 1 to 4.
Double or multiple bonds on Q-C(W^{t-t″}—Rⁱ⁻ⁱⁱⁱ)₃ or Q-C(W^{t-t′}Rⁱ⁻ⁱⁱ)₂R or Q-C(═W¹⁻³)R can be generated also from other substituents, which constitute Q-C(W^{t-t″}Rⁱ⁻ⁱⁱⁱ)₃ or Q-C(W^{t-t′}Rⁱ⁻ⁱⁱⁱ)₂R or Q-C(═W¹⁻³)R under conditions of irradiation (include thermic) or electrochemical reactions, e.g. tetrazenes, cyclic azides, triazenes, dixandionues etc.
   c) or by addition and subsequent reduction on an intermediate R^{t}—C(═W^{t′})(CZ¹Z²)_{f}C(Y^{t})(W^{t}) or independently by step-reaction and subsequent reduction or in situ reduction on R^{t}—C(═W^{t′})(CZ¹Z²)_{f}C(Y¹)(W¹), R—C(═W^{t′})(CZ³Z⁴)_{f}C(Y²)(W²) and R—C(═W^{t′})(CZ⁵Z⁶)_{f}C(Y³)(W³) of structure:

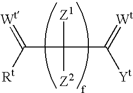

wherein R^{t} is independently group of the same type as R¹⁻², f is 0 or 1, t and t′ is 1 or 2 or 3.
or by addition on intermediates R^{t}—C(═W^{t′})(CZ¹Z²)_{f}C(W^{t-t′}Rⁱ⁻ⁱⁱⁱ)₃ or R^{t}—C(═W^{t′})(CZ¹Z²)_{f}C(W^{t-t′}Rⁱ⁻ⁱⁱⁱ)₂R or R^{t}—C(═W^{t′})(CZ¹Z²)_{f}C(═W¹⁻³)R and by subsequent reduction or in situ reduction or by subsequent hydrolytic, oxidative, reduction cleavage of structure:

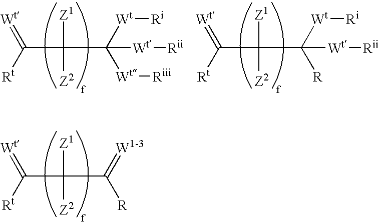

wherein R^{t} is independently group of the same type as R¹⁻², Rⁱ⁻ⁱⁱⁱ is independently group of the same type as R¹⁻², t, t′ and t″ is 1 or 2 or 3, f is 0 or 1.

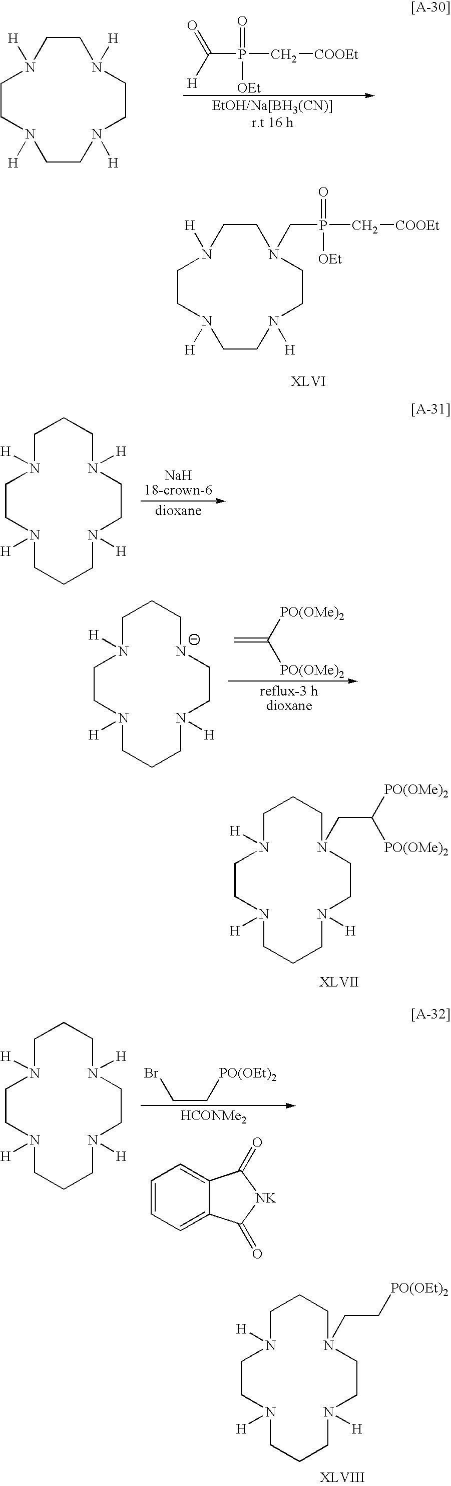

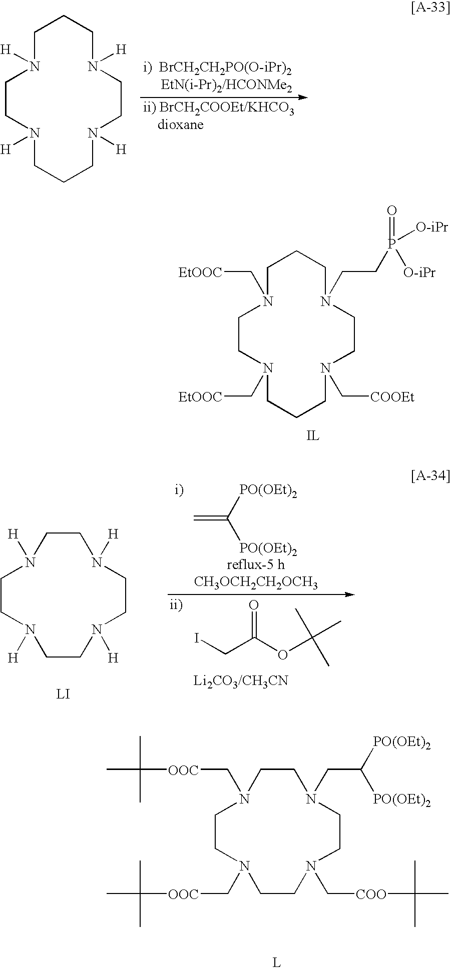

Compounds XLVI-L of the invention can be synthesized based on schemes A-30-A-34. In all cases, intermediates with uniprotective functionalities are used or uniprotective derivatives are targeted compounds of the invention directly.

Either cyclene or cyclame are commercially available. Process of selective monosubstitution is subject of the invention. Suitable intermediates XLVI-L may be synthesized by reaction of cyclone or cyclame or appropriate amide with alkylating reagents containing double bond (scheme A-31), in-situ prepared double bond (scheme A-32). Thus, e.g. diethyl 2-bromoethylphosphonate (scheme A-32) can be applied in case of cyclene. Tetraethyl ethylene-1,1-diphosphonate (scheme A-31) or diisopropyl 2-bromoethylphosphonate (scheme A-33) can be applied in case of cyclame. Special method of this group is reduction alkylation by scheme A-30. The reaction is carried out in presence of cyanoborohydride or other hydride systems. Cyanoborohydride affords excellent yield (94%) of monoalkylated product.

Alkylations by schemes A-33 and A-34, can be carried out by large number of methods of alkylations on secondary amines. In case of alcoxycarbonylmethylation of Li by tert.-butyl ester iodoacetic acid (scheme A-34) reaction gives high yields of corresponding N-alcoxycarbonylmethylated product generally in mild conditions. In most cases solvent-base system composition is basic attribute of this method.

A still further synthetic method of the present invention is the production of the polyazamacrocyclic derivatives of formula (1) via an intermediate which already contains either the phosphorus or arsenic ligand, respectively, or which contains a protective group on one of the nitrogen atoms of the polyazamacrocycle intermediate compound. According to such variant, a reaction of the intermediate already containing the phosphorus or arsenic ligand, respectively, is carried out with a compound which enables the binding of the three —X¹⁻³C(═W¹⁻³)Y¹⁻³ groups to the stiff free nitrogen atoms of the polyazamacrocycle intermediate compound. Alternatively, an intermediate can be taken as a basis which contains one protective group on one of the nitrogen atoms of the polyazamacrocycle. In that case, the three —X¹⁻³C(═W¹⁻³)Y¹⁻³ ligands are attached to the free nitrogen atoms in a first reaction step and, then, by a second reaction, the protective group is removed and the phosphorus or arsenic ligand, respectively, is coupled to the fourth nitrogen atom (process variant ix).

Process variant ix is described in detail in the following.

ix Synthesis of Compounds of Formula (1) from Monoprotected Ligand of Structure:

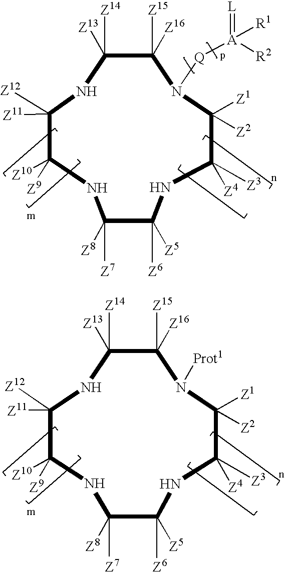

wherein Prot¹ is protective group (or electron pair with negative charge) especially of general structure: —CHO, —COR, —COOR, —CONRⁱRⁱⁱ, —SO₂R, —SR, —R, —SiRⁱRⁱⁱRⁱⁱⁱ, —PORⁱRⁱⁱ, —PSRⁱRⁱⁱ, —PO(ORⁱ)(ORⁱⁱ); wherein Rⁱ⁻ⁱⁱⁱ are groups of the same type as R. Prot¹ is for example methanesulphonyl, 4-toluenesulphonyl, trifluoromethanesulphonyl, nitrobenzenesulphony), benzenesulphonyl, naphthalenesulphonyl, formyl, acetyl, benzoyl, phthaloyl, trifluoroacetyl, tert.-butoxycarbonyl (Boc), 9H-fluoren-9-yl-methoxycarbonyl (Fmoc), benzyloxycarbonyl (Z), ethoxycarbonyl (Eoc), methoxycarbonyl (Meoc), methoxybenzylcarbonyl (Moz), trityl, benzyl, benzhydryl, 4,4-dimethoxytrityl, 4-methoxybenzoyl, ethandioyl, propandioyl, carbonyl, thiocarbonyl etc.
by reaction (e.g. nuceleofilic substitution, addition)
   a) with 3 moles of reactant Subst-(X^{t})C(Y^{t})(W^{t}) (t is 1 or 2 or 3) or independently by step reactions with Subst-(X¹)C(Y¹)(W¹), Subst-(X²C(Y²)(W²) and Subst-(X²)C(Y²)(W²)) of structure:

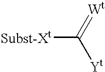

wherein Subst is general leaving group, of structure: —OR, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, wherein Rⁱ⁻ⁱⁱⁱ are groups of the same type as R. (Subst is for example: hydroxyle, alcoxyl, aryloxyl, alkyl amine, N,N-dialkylamine, N-alkylamine, arylsulfonyloxy, tosyloxy, mesyloxy, triflyloxy, acetoxy, benzoyloxy, N-benztriazolyl, trialkylsilyloxy, hydrazine and N-substituted hydrazine, benzyloxycarbonyloxy, tert.-butyloxycarbonyloxy, 1-imidazolyl, succinimidyloxy, N-succinimidyl, N-phthalimidyl, N-phthalimidyloxy, arylthio, thiole, S-alkylthiole etc.), or group which generates in situ or on introduced functional group (X^{t})C(Y^{t})(W^{t}) with or without isolation cation or partial positive charge with capability to reaction (photochemically, thermically or electrochemically cleavable groups)
or by reaction with an intermediate Subst-(CZ¹Z²)ₙCN or by reaction with an intermediate generated in situ by general reaction of HCN and Z¹-C(=L)-Z² and by next hydrolysis with or without isolation of structure:

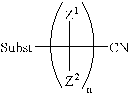

wherein n is 1 or 2,
or by reaction with intermediates Subst-(CZ¹Z²)ₙC(W^{t-t″}Rⁱ⁻ⁱⁱⁱ)₃ or Subst-(CZ¹Z²)ₙC(W^{t-t′}Rⁱ⁻ⁱⁱ)₂R or Subst-(CZ¹Z²)ₙC(═W¹⁻³)R or —(CZ¹Z²)(CZ³Z⁴)W¹⁻³— and by next hydrolytic, oxidative, reduction cleavage of structure:

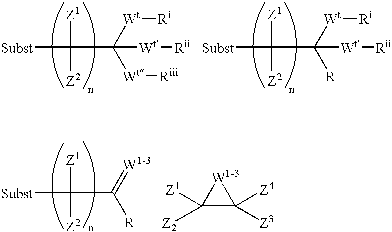

wherein Rⁱ⁻ⁱⁱⁱ is independently group of the same type as R¹⁻², t, t′ and t″ is 1 or 2 or 3, n is 1 or 2.
   b) by addition on an intermediate Q-C(Y^{t})(W^{t}) (t is 1 or 2 or 3) or independently by step-reaction with Q-C(Y¹)(W¹)), Q-C(Y²)(W²) and Q-C(Y³)(W³) of structure:

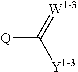

wherein anywhere on Q is double or multiple bond with capability to add intermediates under conditions of general addition: especially carried out under high-pressure (for example in autoclave), microwave irradiation, under reflux in high boiling solvents, in micellar systems, in solid phase, under cryogenic conditions, under phase transfer catalysis, etc.
Double or multiple bonds can be generated in situ with or without isolation by general elimination methods from (Q-(XY)ₙ)—(X^{t})C(Y^{t})(W^{t}) by elimination of XY, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc.
   n is from 1 to 4.
Double or multiple bonds on (Q)-(X^{t})C)(Y^{t})(W^{t}) can be generated also from other substituents, which constitute (Q)-(X^{t})C(Y^{t})(W^{t}) under conditions of irradiation (include thermic) or electrochemical reactions, e.g. tetrazenes, cyclic azides, triazenes, dixandiones etc.
or by addition with an intermediate Q-CN and by next hydrolysis with or without isolation of structure:

Q-CN
Double or multiple bonds can be generated in situ with or without isolation by general elimination of XY from (Q-(XY)ₙ)—CN, wherein:
XY is thermodynamically stable compound capable to elimination, especially: nitrogen, sulphur, ammonia, water, hydrogen sulphide, hydrogen halogenide, metal halogenide, hydrogen or metal alkyl or arylcarboxylates, hydrogen or metal sulphonate or substituted sulphonate, etc.
   n is from 1 to 4.
Double or multiple bonds on Q-CN can be generated also from other substituents, which constitute Q-CN under conditions of irradiation (include thermic) or electrochemical reactions, e.g. tetrazenes, cyclic azides, triazenes, dixandiones etc.
or by addition on intermediates Q-C(W^{t-t″}Rⁱ⁻ⁱⁱⁱ)₃ or Q-C(W^{t-t′}Rⁱ⁻ⁱⁱ)₂R or Q-C(═W¹⁻³)R and by next hydrolytic, oxidative, reduction cleavage of structure:

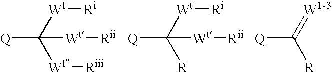

wherein Rⁱ⁻ⁱⁱⁱ is independently group of the same type as R¹⁻², t, t′ and t″ is 1 or 2 or 3.
Double or multiple bonds can be generated in situ with or without isolation by general elimination of XY from (Q-(XY)ₙ)—C(W^{t-t″}Rⁱ⁻ⁱⁱⁱ)₃, or (Q-(XY)ₙ)—C(W^{t-t′}Rⁱ⁻ⁱⁱ)₂R or (Q-(XY)ₙ)—C(═W¹⁻³)R, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc.
n is from 1 to 4.
Double or multiple bonds on Q-C(W^{t-t″}Rⁱ⁻ⁱⁱⁱ)₃ or Q-C(W^{t-t′}Rⁱ⁻ⁱⁱ)₂R or Q-C(═W¹⁻³)R can be generated also from other substituents, which constitute Q-C(W^{t-t″}Rⁱ⁻ⁱⁱⁱ)₃ or Q-C(W^{t-t′}Rⁱ⁻ⁱⁱ)₂R or Q-C(═W¹⁻³)R under conditions of irradiation (include thermic) or electrochemical reactions, e.g. tetrazenes, cyclic azides, triazenes, dixandiones etc.
   c) or by addition and subsequent reduction on an intermediate R^{t}—C(═W^{t′})(CZ¹Z²)_{f}C(Y^{t})(W^{t}) or independently by step-reaction and subsequent reduction or in situ reduction on R—C(═W^{t′})(CZ¹Z²)_{f}C(Y¹)(W¹), R—C(═W^{t′})(CZ³Z⁴)_{f}C(Y²)(W²) and R—C(═W^{t′})(CZ⁵Z⁶)_{f}C(Y³)(W³) of structure:

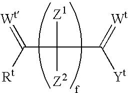

wherein R^{t} is independently group of the same type as R¹⁻², f is 0 or 1, t and t′ is 1 or 2 or 3.
or by addition on intermediates R^{t}—C(═W^{t′})(CZ¹Z²)_{f}C(W^{t-t″}Rⁱ⁻ⁱⁱⁱ)₃ or R^{t}—C(═W^{t′})(CZ¹Z²)_{f}C(W^{t-t′}Rⁱ⁻ⁱⁱ)₂R or R^{t}—C(═W^{t′})(CZ¹Z²)_{f}C(═W¹⁻³)R and by subsequent reduction or in situ reduction or by subsequent hydrolytic, oxidative, reduction cleavage of structure:

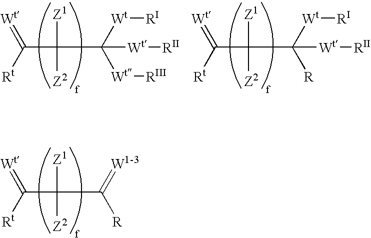

wherein R^{t} is independently group of the same type as R¹⁻², Rⁱ⁻ⁱⁱⁱ is independently group of the same type as R¹⁻², t, t′ and t″ is 1 or 2 or 3, f is 0 or 1.

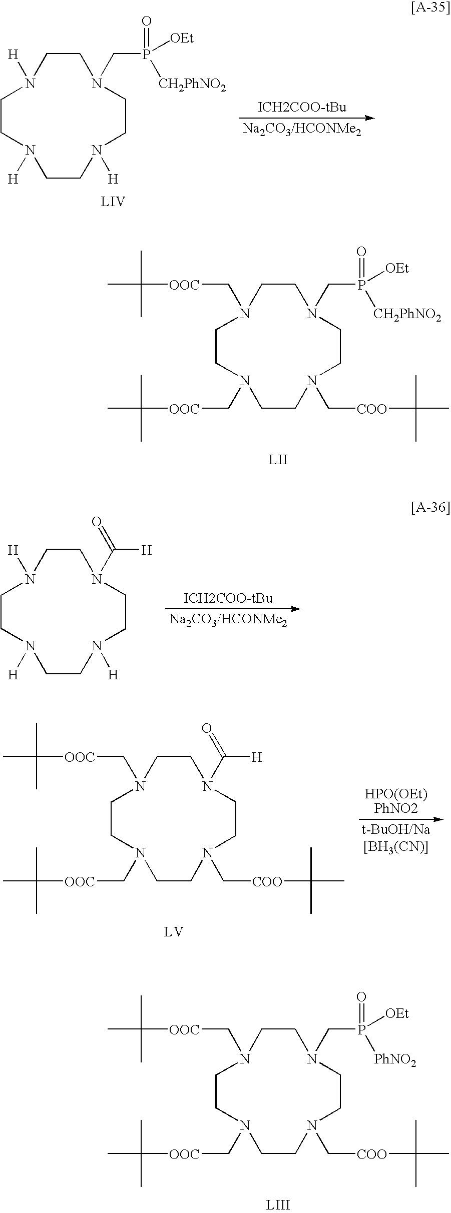

Compounds LII-LIII of the invention can be synthesized based on schemes A-35-A-36. In both cases, intermediates with uniprotective functionalities are used.

Cyclene is well commercially available starting compound. Process of selective monosubstitution is subject of the invention. Suitable intermediates LIV-LV may be synthesized by reaction of cyclene with alkylating reagents. Thus, e.g. tri-tert.-butoxycarbonylmethylation of uniprotected cyclene was carried out by action of tert.-butyl iodoacetate in case of N-monoformylcyclene (scheme A-36) or ethyl 4-nitrobenzylphosphinomethylated cyclene (scheme A-35). Special method for preparation of compounds LII-LIII of the invention is reduction alkylation by scheme A-36. The reaction is carried out in presence of cyanoborohydride or other hydride systems. The method gives good yield (72%) in very mild conditions (24 hours at 0-40° C. in tert.-butanol).

Due to very low lipofilicity, many uniprotected cyclene derivatives are not suitable for execution of reaction under conditions of phase transfer catalysis in two-phase systems containing water and immiscible second solvent. However, in case of high lipofilicity of reaction product this is excellent method for production of these derivatives. In some cases, very good solvent also may be water.

According to the present invention the polyazamacrocycles also can be produced by using an intermediate, wherein all four nitrogen atoms of the polyazamacrocycle are protected (tetraprotected intermediate). According to said process variant, the tetraprotected intermediate is reacted with a compound containing the phosphorus or arsenic ligand, respectively, bound to a leaving group, whereby nucleophilic substitution on one of the nitrogen atoms of the polyazamacrocycle takes place as a result of cleavage of the leaving group. Subsequently, the protective groups are cleaved, and the —X¹⁻³C(═W¹⁻³)Y¹⁻³ ligands are attached (process variant x).

Process variant x is described in detail below.

x) Synthesis of Compounds of Formula (1) from Tetraprotected Ligands N₄(CRⁱCRⁱⁱ), N₄M, N₄Pg¹Pg², N₄Pg¹Prot¹Prot² of Structure:

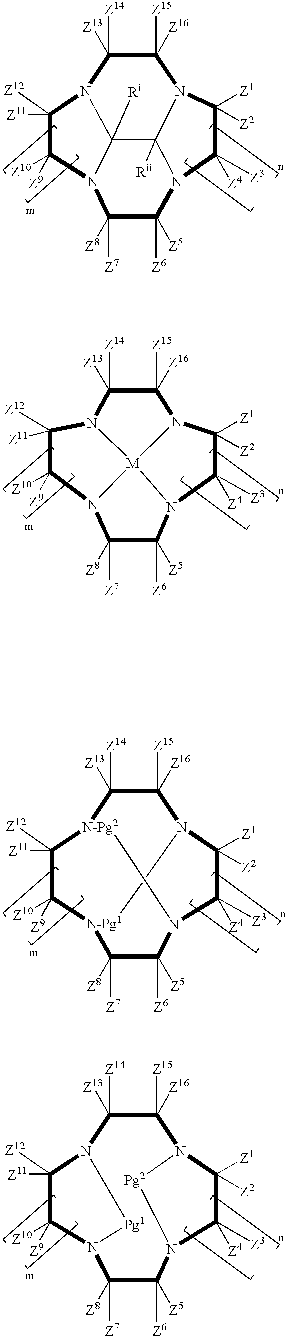

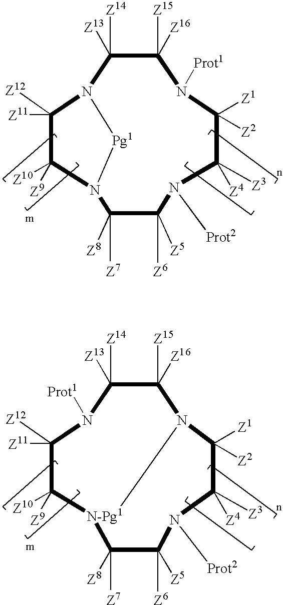

wherein Rⁱ and Rⁱⁱ are groups of the same type as R; M is PR, P—SR, P-halogen, P—OR, silicon, carbon;
Pg¹⁻² is independently protective group especially of structure: CRⁱRⁱⁱ, SiRⁱRⁱⁱ, SnRⁱRⁱⁱ, CO, CS, C(═NR), PO(OR), PS(OR), PO(R), PS(R);
Prot¹ and Prot² is independently protective group (or electron pair with negative charge) especially of general structure: —CHO, —COR, —COOR, —CONRⁱRⁱⁱ, —SO₂R, —SR, —R, —SiRⁱRⁱⁱRⁱⁱⁱ, —PORⁱRⁱⁱ, —PSRⁱRⁱⁱ, —PO(ORⁱ)(ORⁱⁱ) protective groups Prot¹ and Prot² may be also connected to each other especially according to following general structure: —CRⁱRⁱⁱ—, —CO—, —COCO—, —CS—, —C(═NR)—, —COCRⁱRⁱⁱCO—, —CO—R—CO—, [—CRⁱRⁱⁱCRⁱRⁱⁱ]₂SO, [—CRⁱRⁱⁱCRⁱRⁱⁱ]₂SO₂, [—CRⁱRⁱⁱCRⁱRⁱⁱ]₂P(O)OR, [—CRⁱRⁱⁱCRⁱRⁱⁱ]₂NR, —PO(OR)—, —SiRⁱRⁱⁱ—, —SnRⁱRⁱⁱ— wherein Rⁱ⁻ⁱⁱⁱ are groups of the same type as R. Prot¹⁻² is for example methanesulphonyl, 4-toluenesulphonyl, trifluoromethanesulphonyl, nitrobenzenesulphonyl, benzenesulphonyl, naphthalenesulphonyl, formyl, acetyl, benzoyl, phthaloyl, trifluoroacetyl, tert.-butoxycarbonyl (Boc), 9H-fluoren-9-yl-methoxcarbonyl (Fmoc), benzyloxycarbonyl (Z), ethoxycarbonyl (Eoc), methoxycarbonyl (Meoc), methoxybenzylcarbonyl (Moz), trityl, benzyl, benzhydryl, 4,4′-dimethoxytrityl, 4-methoxybenzoyl, ethandioyl, propandioyl, carbonyl, thiocarbonyl etc.
by condensation with Subst-(Q)ₚA(L)(R¹)(R²) of structure;

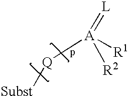

wherein Subst is general leaving group, of structure: —OR, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱⁱ)(SO₂Rⁱⁱ), —NSO₂R, -halogene, —NRⁱNRⁱⁱRⁱⁱⁱ, —SR, —SO₃H or —SO₂Y¹⁻³, wherein Rⁱ⁻ⁱⁱⁱ are groups of the same type as R. (Subst is for example: hydroxyle, alcoxyl, aryloxyl, alkyl amine, N,N-dialkylamine, N-alkylamine, arylsulfonyloxy, tosyloxy, mesyloxy, triflyloxy, acetoxy, benzoyloxy, N-benztriazolyl, trialkylsilyloxy, hydrazine and N-substituted hydrazine, benzyloxycarbonyloxy, tert.-butyloxycarbonyloxy 1-imidazolyl, succinimidyloxy, N-succinimidyl, N-phthalimidyl, N-phthalimidyloxy, arylthio, thiole, S-alkylthiole etc.), or group which generates in situ or on introduced functional group (Q)A(L)(R¹)(R²) with or without isolation cation or partial positive charge with capability to reaction (photochemically, thermically or electrochemically cleavable groups)
under conditions of general nucleofilic substitution: especially under conditions of phase-transfer catalysis, in aprotic polar solvents or its mixtures (as dimethylformamide or dimethylacetamide acetonitrile, dimethylsulphoxide or sulpholane or hexamethylphosphortriamide), in micellar medium, in solidphase (for example bonded N₄G⁻ on anex), with or without microwave irradiation, with or without ultrasonic irradiation, under conditions of high pressure (for example in autoclave), in aqueous phase in presence of pH-buffer, in milieu of water-free solvents with or without presence of base (for example: amines, aldimines, anex, carbonates, fluorides, thioethers), enzymatic catalysis, in presence of dehydrating agent or agent reacting with protogenic product reaction or in presence of Lewis acid as catalysators (for example ZnCl₂, BF₃.Et₂O, SiCl₄) etc.
with or without separation of quaternary monosubstituted ligand from reaction mixture or pentacoordinated N₄M-(Q)ₚA(L)(R¹)(R²) phosphorane.
and by next possible partially or full cleaving of >CRⁱRⁱⁱ and >CRⁱⁱⁱCR^{iv}< bridges or protective groups Prot¹⁻² or Pg¹⁻². Both the steps can be solved as one-step reaction or separately and also by isomerisation of pentacoordinated N₄M-(Q)ₚA(L)(R¹)(R²) phosphorane to N₄G(Q)ₚA(L)(R¹)(R²) all in the conditions described by method in i).

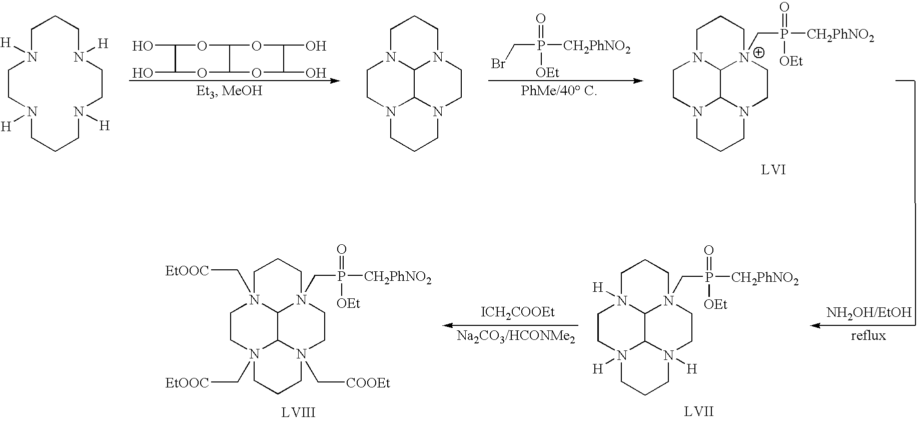

Compound LVIII of the invention can be synthesized based on scheme A-37. Cyclame is well commercially available starting compound. Process of selective internal protection is suitable to carry out by literature method and next quarterization by alkylating reagent (ethyl 4-nitrobenzylbromomethylphosphinate). Ethoxycarbonylmethylation of LVII is executed by ethyl iodoacetate in presence of sodium carbonate—dimethylformamide system after deprotection by action of hydroxylamine on LVI in refluxing ethanol. Due to very low lipofilicity, many uniprotected cyclame derivatives are not suitable for execution of reaction under conditions of phase transfer catalysis in two-phase systems containing water and immiscible second solvent. However, in case of high lipofilicity of reaction product this is excellent method for production of these derivatives.

Another variant of producing the polyazamacrocycles of the present invention starts out from acyclic intermediates. According to such variant, a tertiary amine is reacted with a triamine, whereby condensation leads to the polyazamacrocycle of formula (1) having four nitrogen atoms. The tertiary amine used in said process variant is substituted with the phosphorus or arsenic ligand, respectively, or with a protective group as well as with two bridging members of the polyazamacrocycle (process variant xi). A similar process starts out from an non-cyclic intermediate, whereby, however, the phosphorus or arsenic ligand, respectively, is not bound on the tertiary amine but is located on the triamine (process variant xii).

Process variants xi and xii are described in detail in the following.

xi) Synthesis of Compounds of Formula (1) from Uncyclic Intermediate of Structure:

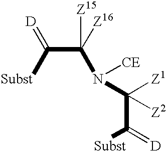

wherein CE is equivalent of (Q)ₚA(L)(R¹)(R²) or Prot¹⁻³ from points i) to viii) from description of ligands; Subst is equivalent leaving group as Subst from points vii) or viii) from description of ligands; D is e.g. oxygene, hydrogen pair, N-substituted or unsubstituted nitrogene, sulphur
by reaction with derivate of structure:

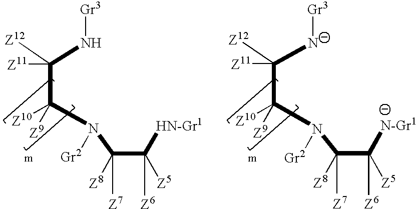

wherein Gr¹⁻³ is group independently equivalent with (X^{t})C(Y^{t})(W^{t}) (t is 1 or 2 or 3) or Prot¹⁻³ from points i) to viii) from description of ligands
under conditions of high dilution, template synthesis, reaction on solid phase, phase transfer catalysis, in aprotic polar solvents, with or without microwave irradiation, with or without presence of ultrasonic.

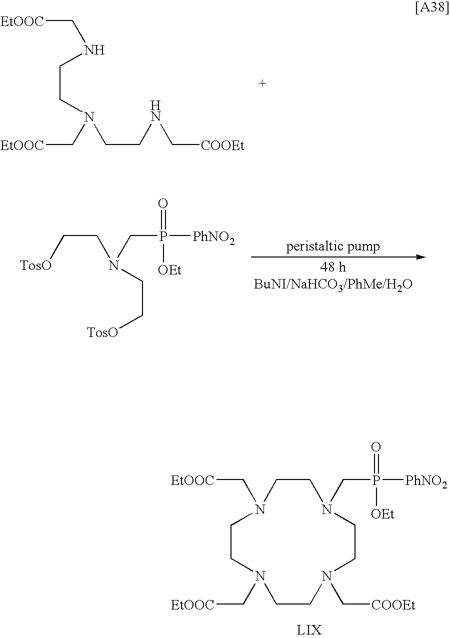

Compound LIX of the invention can be synthesized based on scheme A-38. In this manner, cyclisation is carried out by template effect synthesis or high-dilution effect synthesis generally. Under conditions by the scheme A-38 is used method of template synthesis (presence of sodium) in combination with high dilution method (reaction in toluene and slow adding of both reactants to reaction mixture). The method gives 29% yield of pure cyclene derivative.

xii) Synthesis of Compounds of Formula (1) from Uncyclic Intermediate of Structure:

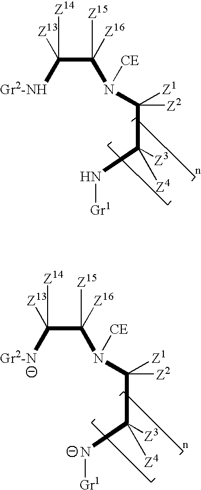

wherein CE is equivalent of (Q)_{pA}(L)(R¹)(R²) or Prot¹⁻³ from points i) to viii) from description of ligands; Gr¹⁻² is group independently equivalent with (X^{t})C(Y^{t})(W^{t}) (t is 1 or 2 or 3) or Prot¹⁻³ from points i) to viii) from description of ligands
by reaction with derivate of structure:

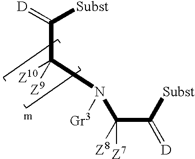

wherein Subst is equivalent leaving group as Subst from points vii) or viii) from description of ligands; Gr³ is group independently equivalent with (X^{t})C(Y^{t})(W^{t}) (t is 1 or 2 or 3) or Prot¹⁻³ from points i) to viii) from description of ligands; D is e.g. oxygene, hydrogen pair, N-substituted or unsubstituted nitrogene, sulphur
under conditions of high dilution, template synthesis, reaction on solid phase, phase transfer catalysis, in aprotic polar solvents, with or without microwave irradiation, with or without presence of ultrasonic.

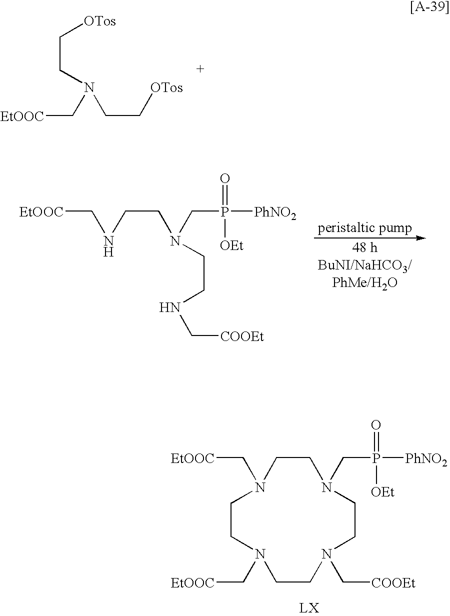

Compound LX of the invention can be synthesized based on scheme A-39. In this manner, cyclisation is carried out by template effect synthesis or high-dilution effect synthesis generally. Under conditions by the scheme A-39 is used method of template synthesis (presence of sodium) in combination with high dilution method (reaction; in toluene and slow adding of both reactants to reaction mixture). The method gives 18% yield of pure cyclene derivative.

A further process variant according to the present invention which involves synthesis via a non-cyclic intermediate starts out from a triamine condensating with a primary amine substituted with the phosphorus or arsenic ligand, respectively, or with a protective group. Thereby the two outer amino groups of the triamine are substituted with a bridging moiety of the polyazamacrocycle, a leaving group or a reactive multiple bond, respectively. According to said process variant an analogous reaction can be effected by the phosphorus or arsenic ligand, respectively, being present on the middle nitrogen atom of the triamine (process variants xii and xiv).

Process variants xiii and xiv are described in detail below:

xiii) Synthesis of Compounds of Formula (1) from Uncyclic Intermediate of Structure:

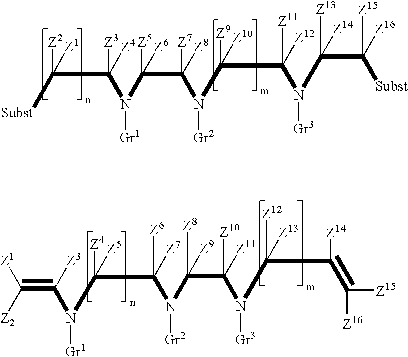

wherein Gr¹⁻³ is group independently equivalent with (X^{t})C(Y^{t})(W^{t}) (t is 1 or 2 or 3) or Prot¹⁻³ from points i) to viii) from description of ligands, Subst is equivalent leaving group as Subst from points vii) or viii) from description of ligands; Gr³ is group independently equivalent with (X^{t})C(Y^{t})(W^{t}) (t is 1 or 2 or 3) or Prot¹⁻³ from points i) to viii) from description of ligands; n, m is independently 1 or 2, nn is 0 or 1;
by reaction with derivate of structure:

H₂N—CE

wherein CE is equivalent of (Q)A(L)(R¹)(R²) or Prot¹⁻³ from points i) to viii) from description of ligands;
under conditions of high dilution, template synthesis, reaction on solid phase, phase transfer catalysis, in aprotic polar solvents, with or without microwave irradiation, with or without presence of ultrasonic.

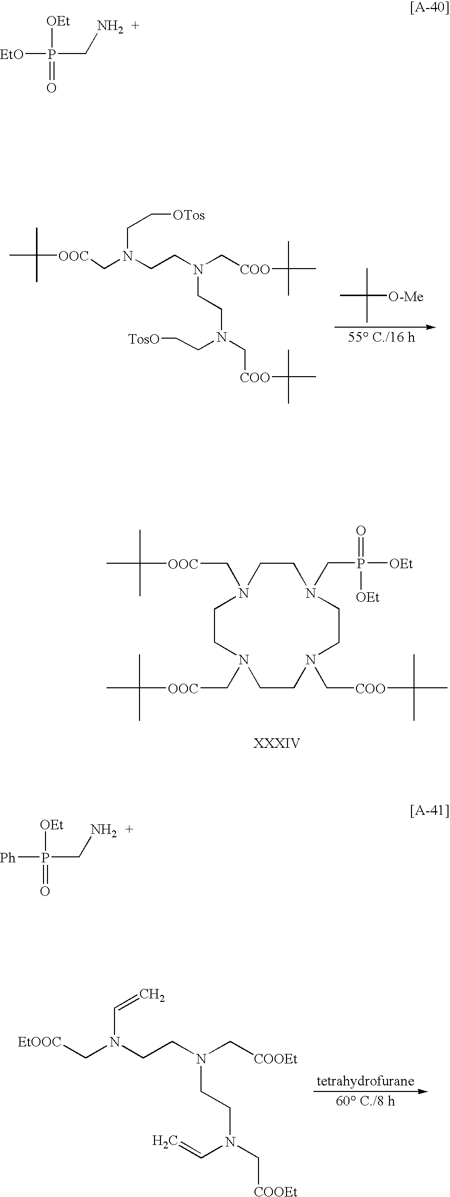

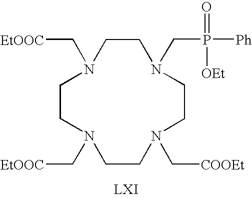

Compounds XXXIV and LXI of the invention can be synthesized based on schemes A-40-A41. In both manners, cyclisation is carried out by high-dilution effect synthesis. The best results are obtained in presence of aprotic ethereal solvents as dioxane, tetrahydrofurane, glyme or methyl tert.-butylether. Reaction conditions are generally similar. Enhance of the yields can be achieved in supradiluted solutions. The method gives 17% yield of pure cyclene derivative XXXIV and 24% of product LXI.

xiv) Synthesis of Compounds of Formula (1) from Uncyclic Intermediate of Structure:

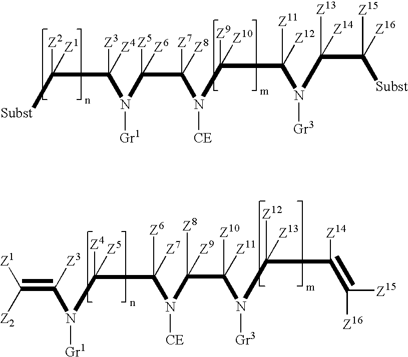

wherein CE is equivalent of (Q)A(L)(R¹)(R²) from points i) to viii) from description of ligands; Gr¹⁻² is group independently equivalent with (X^{t})C(Y^{t})(W^{t}) (t is 1 or 2 or 3) or Proton from points i) to viii) from description of ligands; Subst is equivalent leaving group as Subst from points vii) or viii) from description of ligand; n, m is independently 1 or 2, nn is 0 or 1;
by reaction with derivate of structure:

H₂N-Gr³

wherein Gr³ is group independently equivalent with (X^{t})C(Y^{t})(W^{t}) (t is 1 or 2 or 3) or Prot¹⁻³ from points i) to viii) from description of ligands;
under conditions of high dilution, template synthesis, reaction on solid phase, phase transfer catalysis, in aprotic polar solvents, with or without microwave irradiation, with or without presence of ultrasonic.

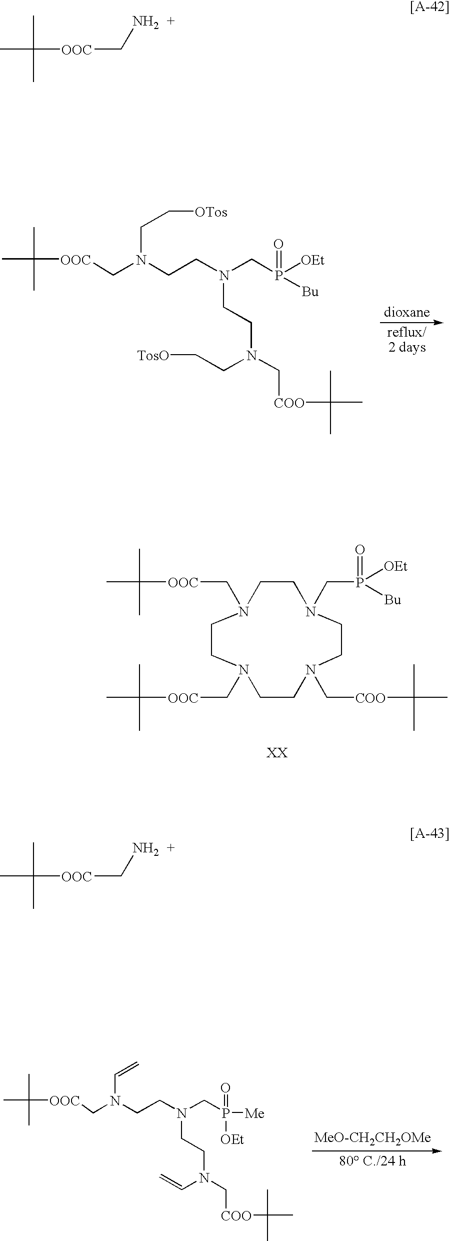

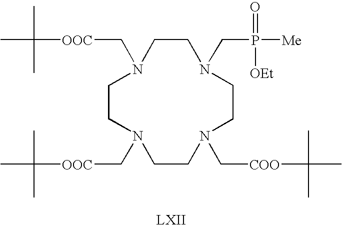

Compounds X) and LXII of the invention can be synthesized based on schemes A-42-A43. In both manners, cyclisation is carried out by high-dilution effect synthesis. The best results are obtained in presence of aprotic ethereal solvents as dioxane, tetrahydrofurane, glyme or methyl tert.-butylether. Reaction conditions are generally similar. Enhance of the yields can be achieved in supradiluted solutions. The method gives 19% yield of pure cyclene derivative XX and 15% of product LXII.

According to another process variant of the present invention, the polyazamacrocycle derivatives of formula (1) are produced by oxidation of the phosphorus or arsenic ligand, respectively, by an oxidant. Oxidation is preferably effected on protected intermediates. Further reactions which, according to the invention, can take place on protected intermediates are addition reactions, alkylation or arylation reactions or substitution to obtain the polyazamacrocycle derivative of formula (1) as the end product (process variant xv).

Process variant xv is described in detail in the following.

xv) Synthesis of Compounds of Formula (1) from Protected Intermediates of Structure:

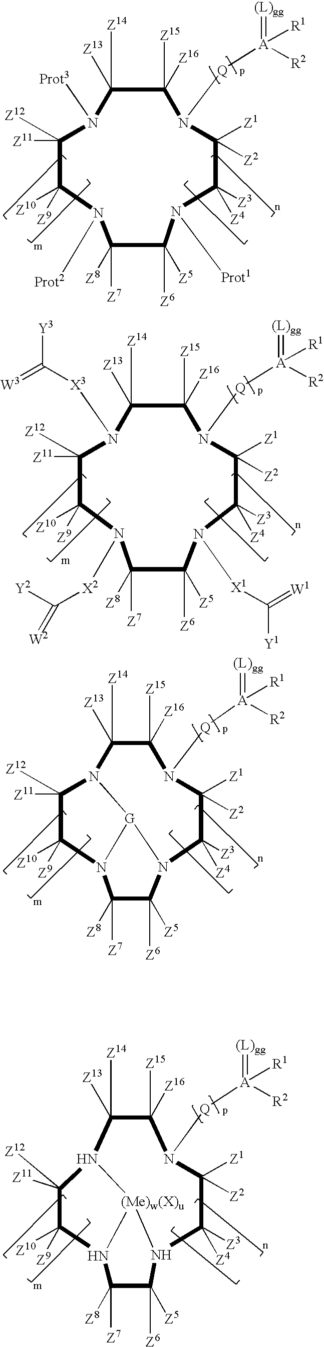

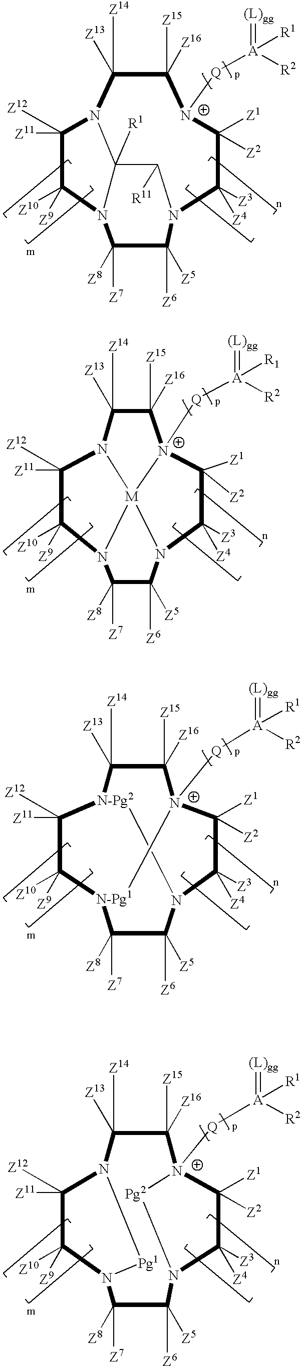

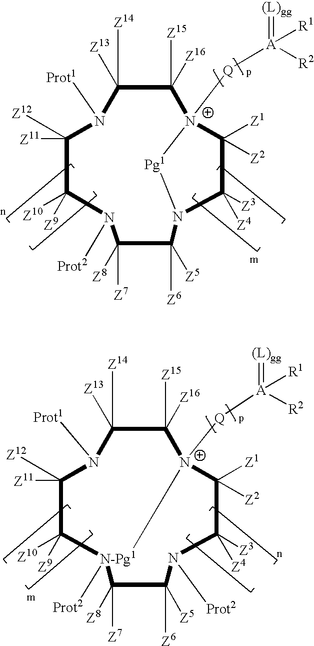

wherein Prot¹⁻³ is independently protective group (or electron pair with negative charge) especially of general structure: —CHO, —COR, —COOR, —CONRⁱRⁱⁱ, —SO₂R, —SR, —R, —SiRⁱRⁱⁱRⁱⁱⁱ, —PORⁱRⁱⁱ, —PSRⁱRⁱⁱ, —PO(ORⁱ)(ORⁱⁱ); protective groups Prot¹ and Prot² or Prot¹ and Prot³ may be also connected to each other especially according to following general structure: —CRⁱRⁱⁱ—, —CO—, —COCO—, —CS—, —C(═NR)—, —COCRⁱRⁱⁱCO—, —CO—R—CO—, [—CRⁱRⁱⁱCRⁱRⁱⁱ]₂SO, [—CRⁱRⁱⁱCRⁱRⁱⁱ]₂SO₂, [—CRⁱRⁱⁱCRⁱRⁱⁱ]₂P(O)OR, [—CRⁱRⁱⁱCRⁱRⁱⁱ]₂NR, —PO(OR)—, —SiⁱRⁱⁱ—, —SnRⁱRⁱⁱ— wherein Rⁱ⁻ⁱⁱⁱ are groups of the same type as R. Prot¹⁻³ is for example methanesulphonyl, 4-toluenesulphonyl, trifluoromethanesulphonyl, nitrobenzenesulphonyl, benzenesulphonyl, naphthalenesulphonyl, formyl, acetyl, benzoyl, phthaloyl, trifluoroacetyl, tert.-butoxycarbonyl (Boc), 9H-fluoren-9-yl-methoxycarbonyl (Fmoc), benzyloxycarbonyl (Z), ethoxycarbonyl (Eoc), methoxycarbonyl (Meoc), methoxybenzylcarbonyl (Moz), trityl, benzyl, benzhydryl, 4,4-dimethoxytrityl, 4-methoxybenzoyl, ethandioyl, propandioyl, carbonyl, thiocarbonyl etc. wherein G is CZ¹⁻¹⁶ (include C⁺ as carbocation with Z¹⁻¹⁶ as anion), SiZ¹⁻¹⁶, SnZ¹⁻¹⁶, B, Al, P, As, PO, AsO, PS, AsS, AsZ¹⁻¹⁶, VZ¹⁻¹⁶, PZ¹⁻¹⁶;
G is CZ¹⁻¹⁶ (include C⁺ as carbocation with Z¹⁻¹⁶ as anion), SiZ¹⁻¹⁶, SnZ¹⁻¹⁶, B, Al, P, As, PO, AsO, PS, AsS, AsZ¹⁻¹⁶, VZ¹⁻¹⁶, PZ¹⁻¹⁶;
Me is metal (or ion), especially: Cu, Ni, Fe, Zn, Cr, Mo, V; X is ligand for example Cl, Br, OH, etc.; u is from 1 to 15; w is 1 or 2 or 3 or ½ or ⅔ or 3/2), or formates TiOZ¹⁻¹⁶, TiNZ¹⁻¹⁶ MoP, MoN;
wherein Rⁱ and Rⁱⁱ are groups of the same type as R; M is PR, P—SR, P-halogen, P—OR, silicon, carbon;
Pg¹⁻² is independently protective group especially of structure: CRⁱRⁱⁱ, SiRⁱRⁱⁱ, SnRⁱRⁱⁱ, CO, CS, C(═NR), PO(OR), PS(OR), PO(R), PS(R),
gg is 0 or 1
where molecular fragments -QₚA(L)_{gg}R¹R² and -QₚA(R¹)(R²)(R³)(R⁴) undergoes to transformations:

### Oxidation, by Scheme:

-QₚA(L)HR¹+oxidant→-QₚA(L)R¹OH

-QₚA(R¹)(R²)+oxidant→-QₚA(L)R¹R²

wherein oxidant is atom or molecule with possibility to oxidation of -QₚA(L)HR¹ or -QₚA(R¹)(R²), e.g. oxygen, sulphur, hydrogen peroxide, hypochlorite, halogens, hexacyanoferrate(III), peroxodisulphate, peroxoborate, chromate and dichromate, permanganate, manganese(IV) dioxide etc.,

### Adition, by Scheme:

-QₚA(L)HR¹+RⁱRⁱⁱC═CRⁱⁱⁱR^{iv→}-QₚA(L)(R²)[C(Rⁱ)(Rⁱⁱ)—C(Rⁱⁱⁱ)(R^{iv})(H)]

-QₚA(L)HR¹+RⁱRⁱⁱC═W^{1-3→}-QₚA(L)(R²)[C(Rⁱ)(Rⁱⁱ)(W′H)]

-QₚA(L)HR¹+RⁱRⁱⁱC═W¹⁻³+reductant→-QₚA(L)(R²)[C(Rⁱ)(Rⁱⁱ)(H)]

-QₚAH(R¹)(R²)(R³)+RⁱRⁱⁱC═CRⁱⁱⁱR^{iv→}-Qₚ[A(R⁴)(R⁵)(R⁶)]C(Rⁱ)(Rⁱⁱ)—C(Rⁱⁱⁱ)(R^{iv})(H)

-QₚAH(R¹)(R²)(R³)+RⁱRⁱⁱC═W^{1-3→}-Qₚ[A(R⁴)(R⁵)(R⁶)]C(Rⁱ)(Rⁱⁱ)(W′H)

-QₚAH(R¹)(R²)(R³)+RⁱRⁱⁱC═W¹⁻³+reductant→-Qₚ[A(R⁴)(R⁵)(R⁶)]C(Rⁱ)(Rⁱⁱ)(H)

-QₚAR¹R²+RⁱRⁱⁱC═CRⁱⁱⁱR^{iv→}-QₚA(L)(R³)[C(Rⁱ)(Rⁱⁱ)]—C(Rⁱⁱ)(R^{iv})(R⁴)

-QₚAR¹R²+RⁱRⁱⁱC═W^{1-3→}-QₚA(L)(R³)[C(Rⁱ)(Rⁱⁱ)(W′R⁴)]

-QₚA(L)HR¹+RⁱRⁱⁱC═W¹⁻³+Le-R^{2→}-QₚA(L)R³[C(Rⁱ)(Rⁱⁱ)(W′R⁴)]

-QₚAH(R¹)(R²)(R³)+RⁱRⁱⁱC═W¹⁻³+LeR^{4→}-QₚA(R⁵)(R⁶)(R⁷)[C(Rⁱ)(Rⁱⁱ)(W′R⁴)]

-QₚAR¹R²+RⁱRⁱⁱC═W¹⁻³+LeR^{3→}-QₚA(L)(R⁴)[C(Rⁱ)(Rⁱⁱ)(W′R³)]

-QₚA(L)R¹(CR²═CR³R⁴)+HW′R⁵→-QₚA(L)(R⁶){[C(R²)(H)]—[C(R³)(R⁴)(W′R⁵)]}

-QₚA(L)R¹(CR²═CR³R⁴)+AR⁵R⁶R⁷→-QₚA(L)(R⁸){[C(R²)(R⁵)]—[C(R³)(R⁴)(AR⁹R¹⁰)]}

-QₚA(L)R¹(CR²═CR³R⁴)+AR⁵R⁶R⁷→-QₚA(L)(R⁸){[C(R²)(R⁵)]—[C(R³)(R⁴)(A(L)R⁹R¹⁰)]}

-QₚA(L)R¹(CR²═CR³R⁴)+HAR⁵R⁶R⁷R⁸→-QₚA(L)(R⁸){[C(R²)(H)]—[C(R³)(R⁴)(AR⁹R¹⁰R¹¹R¹²)]}

QₚA(L)R¹(CR²═CR³R⁴)+HAR⁵R⁶R⁷R⁸→-QₚA(L)(R⁸){[C(R²)(H)]—[C(R³)(R⁴)(A(L)R⁹R¹⁰)]}

wherein Le is leaving group, especially of structure: —OR, —OH, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, N-benztriazolyl, 1-imidazolyl, succinimidyloxy, N-succinimidyl, N-phthalimidyl, N-phthalimidyloxy wherein Rⁱ⁻ⁱⁱⁱ are groups of the same type as R
wherein R^{i-iv} are groups of the same type as R, W′ is independently oxygen, sulphur, NH, NR⁶, A(L)R⁶, AR⁶R⁷R⁸, W¹⁻³;
R³⁻¹² are groups of the same type as R¹⁻²

### Alkylation or Arylation, by Scheme:

-QₚA(L)HR¹+Subst-R²→-QₚA(L)R¹R²

-QₚAR¹R²+Subst-R³→-QₚA(L)R⁴R³

wherein Subst is general leaving group, of structure: —OR, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, wherein Rⁱ⁻ⁱⁱⁱ are groups of the same type as R. (Subst is for example: hydroxyle, alcoxyl, aryloxyl alkyl amine, N,N-dialkylamine, N-alkylamine, arylsulfonyloxy, tosyloxy, mesyloxy, triflyloxy, acetoxy, benzoyloxy, N-benztriazolyl, trialkylsilyloxy, hydrazine and N-substituted hydrazine, benzyloxycarbonyloxy, tert.-butyloxycarbonyloxy 1-imidazolyl, succinimidyloxy, N-succinimidyl, N-phthalimidyl, N-phthalimidyloxy, arylthio, thiole, S-alkylthiole etc.), or group which generates in situ or on introduced functional group R² with or without isolation cation or partial positive charge with capability to reaction (photochemically, thermically or electrochemically cleavable groups); R³⁻⁴ are groups of the same type as R¹⁻²

### Substitution, by Scheme:

-QₚA(L)HR¹→-QₚA(L′)HR¹

-QₚA(L)HR¹→-QₚA(L′)HR²

-QₚA(L)HR¹→-QₚAR²R³

-QₚA(L)HR¹→-QₚAR¹R²

wherein R³ is group of the same type as R¹⁻²

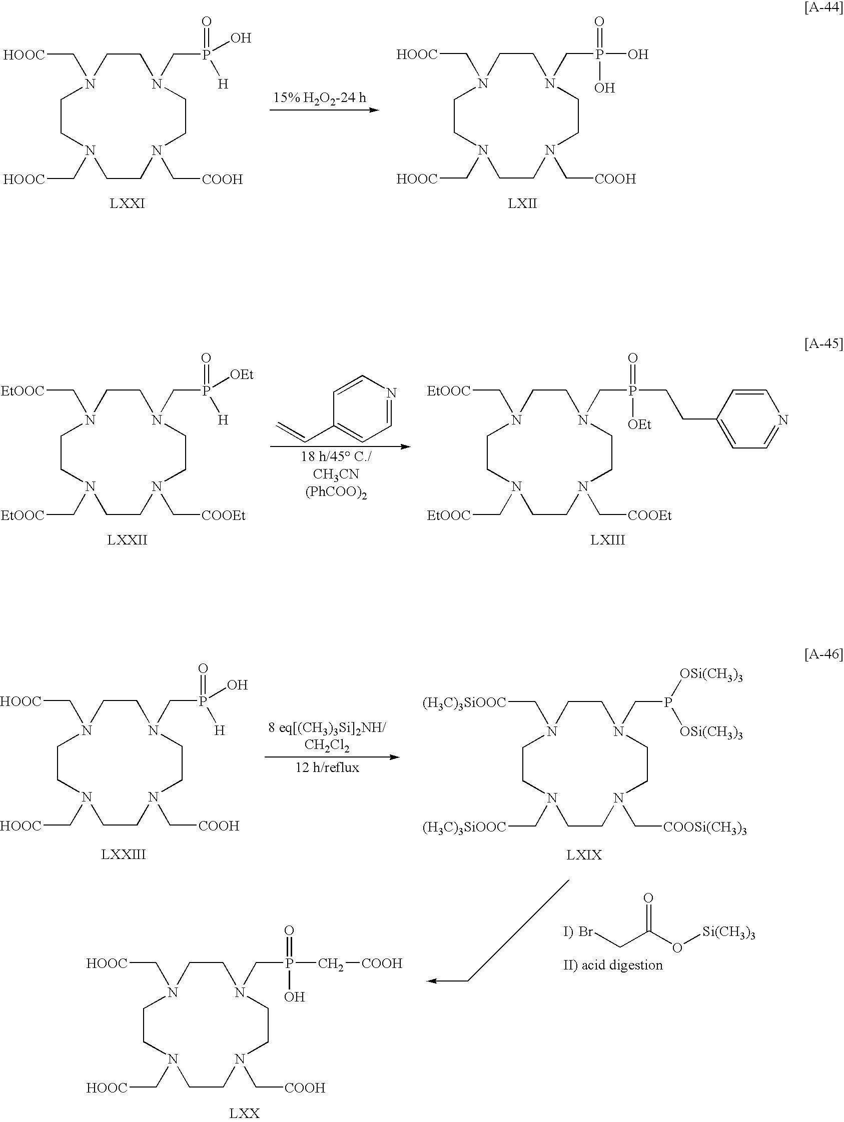

Redox reactions on derivatives based on phosphurus binded to organic molecules are very important synthetic alternatives. Thus, substituted methylphosphinic acid LXXI can be by action of diluted hydrogen peroxide (15%) oxidized to appropriate phosphonic acid in almost quantitative yield (A-45). Separation is carried out in catex column with advantage. Adding of phosphinates to activated double bound is much more difficult. In case of 4-vinylpyridine DBPO catalysis is necessary. 51% of pure separated ethyl dialkylphosphinate LXIII is obtained after stirring at mild conditions. Very important factor is purity of 4-vinylpyridine and acetonitrile. Trimethylsilyl group has high importance in this invention. Hexamethyldisilazane affords in reflux with any phosphinated triprotected azamacrocycles appropriate polytrimethylsilylated derivatives. Thus derivative LXIX has been obtained in case of DO3A-methylenephosphinic acid. Due to acidity of input reactant, no catalysis is necessary. But it is possible. Nevertheless, with no effect to overall yield. Alkylation of pertrimethylsilylated dihydroxyalkylphosphane LXIX with trimethylsilyl ester bromoacetic acid carries out in excellent yield of dialkylphosphinic acid LXX after necessary hydrolysis in alcoholic medium. Total yield of all steps is 49% and therefore this methodics is very suitable to any manufacturing process.

The present invention further concerns a process variant starting out from a polyazamacrocycle intermediate protected by a three-functional ligand (unitriprotected intermediate). Said intermediate is reacted with two compounds which, together, form the phosphorus or arsenic ligand respectively. Subsequently, the three-functional protecting group is cleaved, and the polyazamacrocycle derivative of formula (1) is obtained (process variant xvi).

Process variant xvi is explained in detail below.

xvi) Synthesis of Compounds of Formula (1) from Unitriprotected Intermediates of Structure:

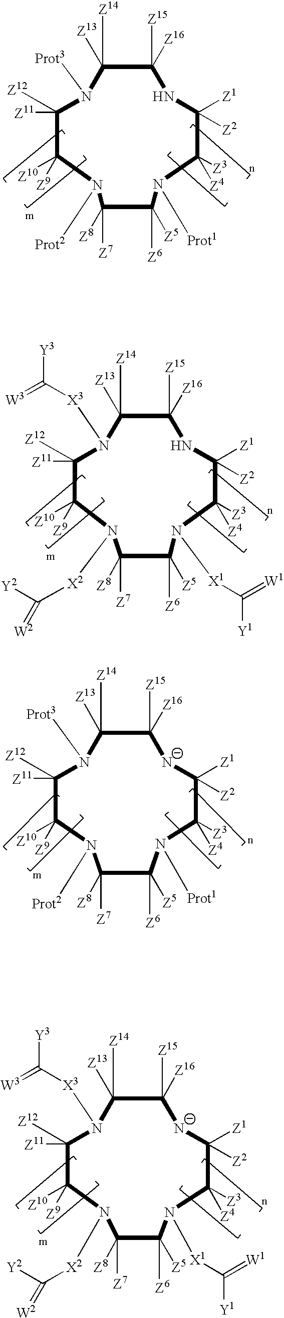

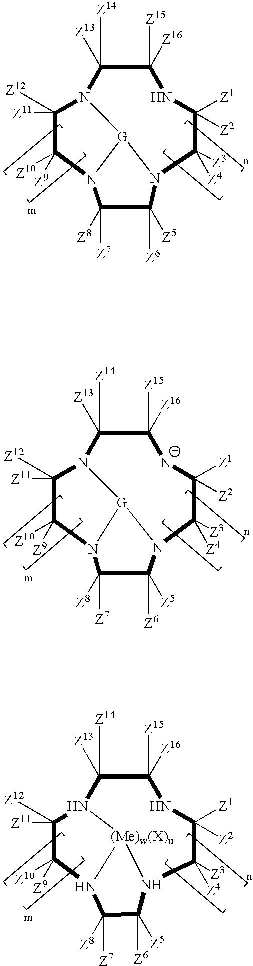

wherein G is CZ¹⁻¹⁶ (include C⁺ as carbocation with Z¹⁻¹⁶ as anion), SiZ¹⁻¹⁶, SnZ¹⁻¹⁶, B, Al, P, As, PO, AsO, PS, AsS, AsZ¹⁻¹⁶, VZ¹⁻¹⁶, PZ¹⁻¹⁶,
Me is metal (or ion), especially: Cu, Ni, Fe, Zn, Cr, Mo, V; X is ligand for example Cl, Br, OH, etc.; u is from 1 to 15, w is 1 or 2 or 3 or ½ or ⅔ or 3/2), or formates TiOZ¹⁻¹⁶, TiNZ¹⁻¹⁶, MoP, MoN;
wherein Prot¹⁻³ is independently protective group (or electron pair with negative charge) especially of general structure: —CHO, —COR, —COOR, —CONRⁱRⁱⁱ, —SO₂R, —SR, —R, —SiRⁱRⁱⁱRⁱⁱⁱ, —PORⁱRⁱⁱ, —PSRⁱRⁱⁱ, —PO(ORⁱ)(ORⁱⁱ); protective groups Prot¹ and Prot² or Prot¹ and Prot³ may be also connected to each other especially according to following general structure: —CRⁱRⁱⁱ—, —CO—, —COCO—, —CS—, —C(═NR)—, —COCRⁱRⁱⁱCO—, —CO—R—CO—, [—CRⁱRⁱⁱCRⁱRⁱⁱ]₂SO, [—CRⁱRⁱⁱCRⁱRⁱⁱ]₂SO₂, [—CRⁱRⁱⁱCRⁱRⁱⁱ]₂P(O)OR, [—CRⁱRⁱⁱCRⁱRⁱⁱ]₂NR, —PO(OR)—, —SiRⁱRⁱⁱ—, —SnRⁱRⁱⁱ— wherein Rⁱ⁻ⁱⁱⁱ are groups of the same type as R. Prot¹⁻³ is for example methanesulphonyl, 4-toluenesulphonyl, trifluoromethanesulphonyl, nitrobenzenesulphonyl, benzenesulphonyl, naphthalenesulphonyl, formyl, acetyl, benzoyl, phthaloyl, trifluoroacetyl, tert.-butoxycarbonyl (Boc), 9H-fluoren-9-yl-methoxycarbonyl (Fmoc), benzyloxycarbonyl (Z), ethoxycarbonyl (Eoc), methoxycarbonyl (Meoc), methoxybenzylcarbonyl (Moz), trityl, benzyl, benzhydryl, 4,4′-dimethoxytrityl, 4-methoxybenzoyl, ethandioyl, propandioyl, carbonyl, thiocarbonyl etc.
by condensation with A(L)(R¹)(R²)(R³), HA(L)(R¹)(R²), A(R¹)(R²)(R³), HA(R¹)(R²), HA(R¹)(R²)(R³)(R⁴) of structure:

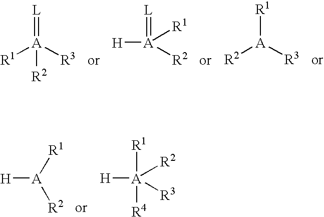

wherein R³ and R⁴ are groups of the same type as R¹⁻²,
with methylene or substituted methylene reactive group structure of aldehyde (e.g. formaldehyde, acetaldehyde, benzaldehyde, 4-N,N-dimethylaminobenzaldehyde, nitrobenzaldehyde, 2-chlorobenzaldehyde, anisaldehyde etc.), aldehyde acetals or semiacetals (e.g. formaldehyde dimethylacetal), chloromethylethers (e.g. chloromethylmethylether), 1,1,1-trialkoxyalkane, diazomethane or C-substituted diazomethanes
under conditions of general condensation: especially under conditions of azeotropic water off distillation, phase-transfer catalysis, in aprotic polar solvents or its mixtures (as dimethylformamide or dimethylacetamide or acetonitrile, dimethylsulphoxide or sulpholane or hexamethylphosphortriamide), in micellar medium, in solidphase (for example bonded N₄G⁻ on anex), with or without microwave irradiation, with or without ultrasonic irradiation, under conditions of high pressure (for example in autoclave), in aqueous phase in presence of pH-buffer, in milieu of water-free solvents with or without presence of base (for example: amines, aldimines, carbonates, fluorides, thioethers), enzymatic catalysis, in presence of dehydrating agent or agent reacting with protogenic product reaction or in presence of Lewis acid (e.g. ZnCl₂, BF₃, Et₂O, SiCl₄) etc.
and if needed, by next partially or full cleaving of G or (Me)_{w}(X)ᵤ. Both the steps can be solved as one-step reaction or separately.

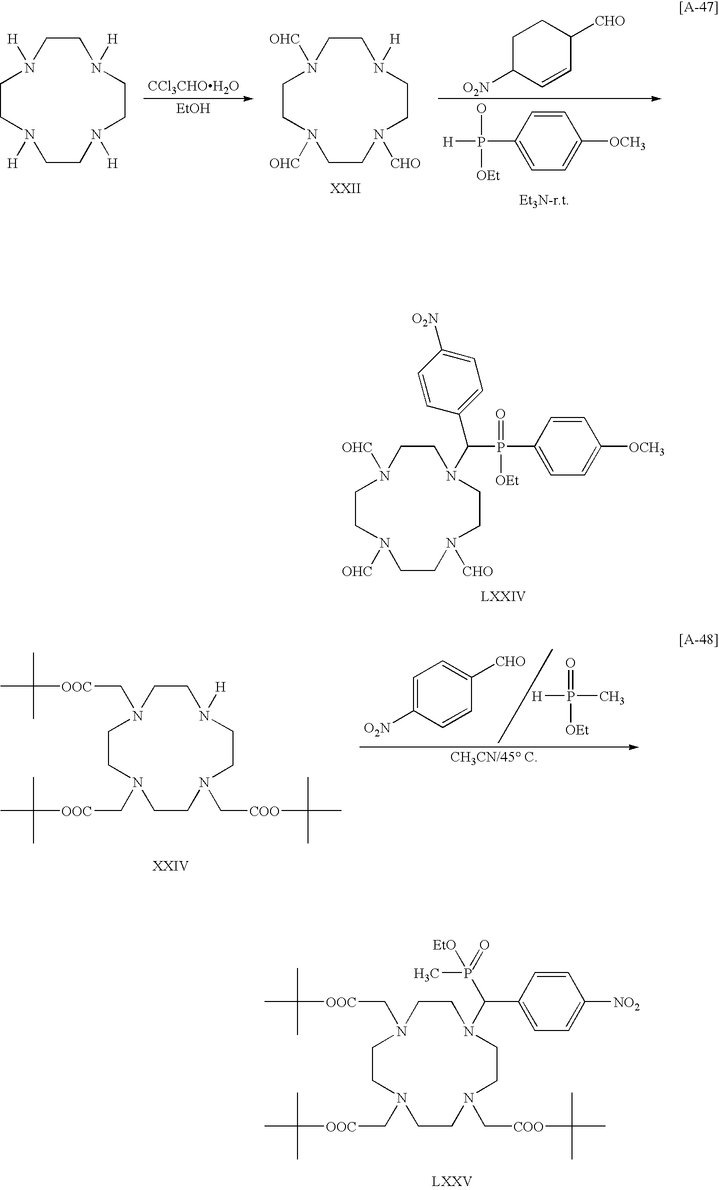

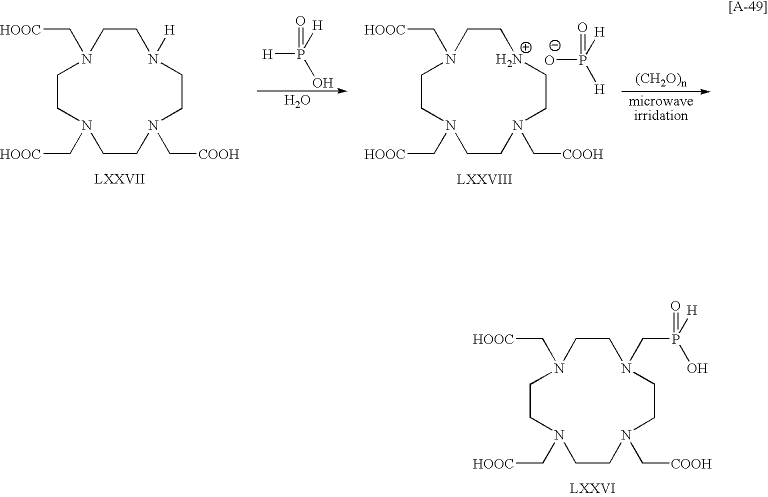

In all cases, starting cyclen is available as commercial product. Triformylcyclene (XXII, A47) can be obtained by procedure described in V. Boldrini, G. B. Giovenzana, R. Pagliarn, G. Palmisano, M. Sisti: Tetraherdon Letters 41 (2000) 6527. Reaction with aldehydes and alkyl esters of aryl(alkyl)phosphinates can be seen in schemes A47, A-48 and A-49. Thus, e.g. ethyl (4-methoxyphenyl)phoshinate (A47), ethyl methylphosphinate (A48) or hypophosphorous acid can be applied (A-49). Facility of carrying out is important aspect of this reaction. Low temperatures, low reaction times and generally mild conditions are necessary. In case of DO3A tri-tert.-butyl ester (XXIV) condensation with 4-nitrobenzaldehyde and ethyl methylphosphinate (A48) product of Mannich's condensation has been obtained by 8 hours stirring of reactants in acetonitrile. Total yield was 46% of separated pure product. In case of hypophosphorous salt of cyclene derivative LXXVII is possible to carry out reaction with convenience by microwave irradiation. Yields are often excellent. But thermodynamic stability of reactants and products is important standpoint. Overall yield of A-49 is 84%. This is very suitable method for direct phosphinomethylation to cyclene sceleton.

Still further, the polyazamacrocycle compounds (1) may be prepared from vinyl-triprotected intermediates. Said intermediates are characterized in that three of the four nitrogen atoms are blocked via protecting groups and that the nitrogen atom carrying the phosphorus ligand is coupled to a vinyl group. The phosphorus ligand is built up on said reactive vinyl group (process variant xvii).

This last synthesis variant xvii is explained in detail below.

xvii) Synthesis of Compounds of Formula (1) from Vinyl-Triprotected Intermediates:
wherein

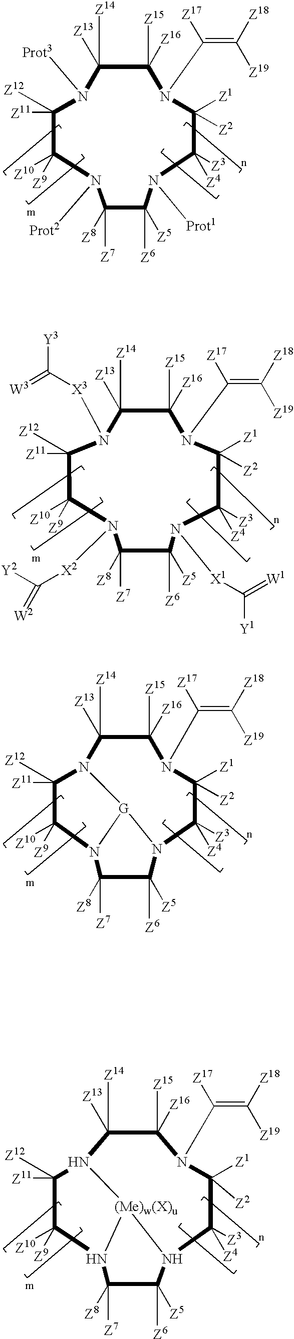

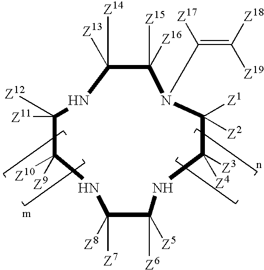

G is CZ¹⁻¹⁶ (include C⁺ as carbocation with Z¹⁻¹⁶ as anion), SiZ¹⁻¹⁶, SnZ¹⁻¹⁶, B, Al, P, As, PO, AsO, PS, AsS, AsZ¹⁻¹⁶, VZ¹⁻¹⁶, PZ¹⁻¹⁶;
Me is metal (or ion), especially: Cu, Ni, Fe, Zn, Cr, Mo, V; X is ligand for example Cl, Br, OH, etc.; u is from 1 to 15; w is 1 or 2 or 3 or ½ or ⅔ or 3/2), or formates TiOZ¹⁻¹⁶, TiNZ¹⁻¹⁶, MoP, MoN;
Z^{17-Z19} are groups of the same type as Z¹⁻¹⁶
Prot¹⁻³ is independently protective group (or electron pair with negative charge) especially of general structure: —CHO, —COR, —COOR, —CONRⁱRⁱⁱ, —SO₂R, —SR, —R, —SiRⁱRⁱⁱRⁱⁱⁱ, —PORⁱRⁱⁱ, —PSRⁱRⁱⁱ, —PO(ORⁱ)(ORⁱⁱ); protective groups Prot¹ and Prot² or Prot¹ and Prot³ may be also connected to each other especially according to following general structure: —CRⁱRⁱⁱ—, —CO—, —COCO—, —CS—, —C(═NR)—, —COCRⁱRⁱⁱCO—, —CO—R—CO—, [—CRⁱRⁱⁱCRⁱRⁱⁱ]₂SO, [—CRⁱRⁱⁱCRⁱRⁱⁱ]₂SO₂, [—CRⁱRⁱⁱCRⁱRⁱⁱ]₂P(O)OR, [—CRⁱRⁱⁱCRⁱRⁱⁱ]₂NR, —PO(OR)—, —SiRⁱRⁱⁱ—, —SnRⁱRⁱⁱ— wherein Rⁱ⁻ⁱⁱⁱ are groups of the same type as R. Prot¹⁻³ is for example methanesulphonyl, 4-toluenesulphonyl, trifluoromethanesulphonyl, nitrobenzenesulphonyl, benzenesulphonyl, naphthalenesulphonyl, formyl, acetyl, benzoyl, phthaloyl, trifluoroacetyl, tert,-butoxycarbonyl (Boc), 9H-fluoren-9-yl-methoxycarbonyl (Fmoc), benzyloxycarbonyl (Z), ethoxycarbonyl (Eoc), methoxycarbonyl (Meoc), methoxybenzylcarbonyl (Moz), trityl, benzyl, benzhydryl, 4,4′-dimethoxytrityl, 4-methoxybenzoyl, ethandioyl, propandioyl, carbonyl, thiocarbonyl etc.
by reaction (e.g. addition) with precursors or their mixture of structure:

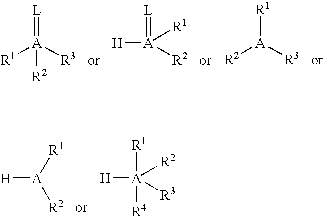

wherein R³ and R⁴ are groups of the same type as R¹⁻². Especially can be used: alkylphosphinic acid, arylphosphinic acid, trialkylphosphites, triarylphosphites, trialkylphosphines, triarylphosphines, dialkylphosphinates, diarylphosphinates, alkylarylphosphinates, dialkylarylphosphites, alkyldiarylphosphites, phosphinic acid, alkylarsenic(III) acid, arylarsenic(III) acid, trialkylarsenic(III), triarylarsenic(III), etc.
under conditions of general addition: especially carried out under high-pressure (for example in autoclave), microwave irradiation, under reflux in high boiling solvents, in micellar systems, in solid phase, under cryogenic conditions, under phase transfer catalysis, etc.

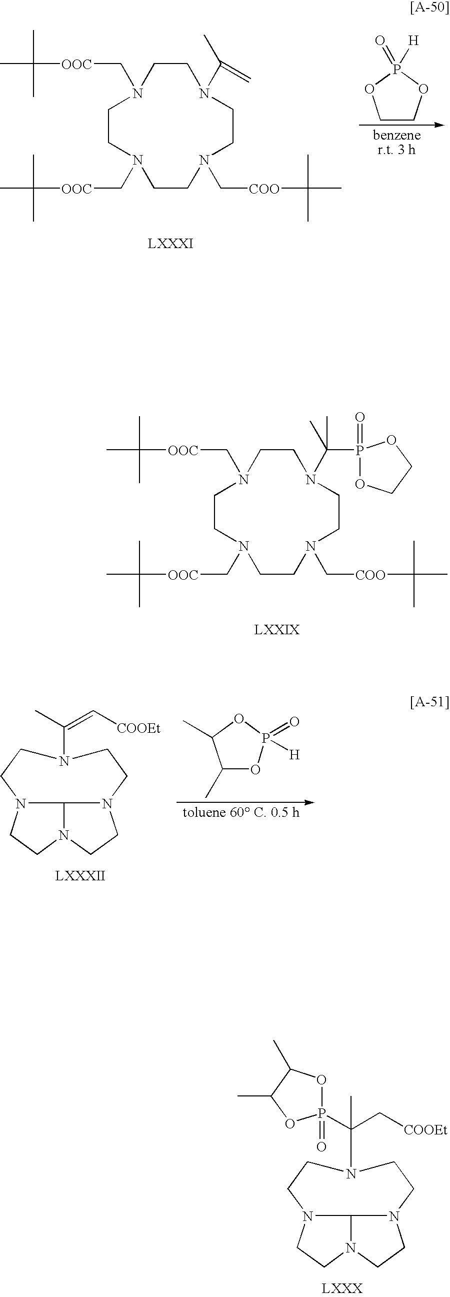

Method of dialkyl phosphite adding to enamines is suitable for simple and high conversion preparation of alkylphosphites with methylene bridge to macrocycle's sceleton. Main advantages are mild reaction conditions and low reaction times respectively. Thus ligand LXXIX can be obtained in 78% yields of separated product by stirring of N-propen-2-ylcyclene derivative LXXXI with cyclic phosphite in benzene for 8 hours (A-50). This method has enormous importance, if there is requirement for substituted chain of methylene's bridge. In similar manner reacts cyclic phosphite with unitriprotected cyclene substituted with trans-crotonic acid LXXXII. Product LXXX is obtained in 88% yield in very short time of tenths minutes.

A further aspect of the present invention relates to novel polyazamacrocycle derivatives being the end product obtained by the synthetic approaches i-xvii of the present invention.

A still further aspect of the present invention concerns the intermediates described in the synthetic methods i-xvii of the present invention.

The invention further relates to a synthetic route for the preparation of triprotected intermediates as described above in synthesis routes iii and vii.

Methods of Preparation or Synthesis of Triprotected Intermediates for Synthesis Ligands from Parts iii, vi of the Structure:

wherein:

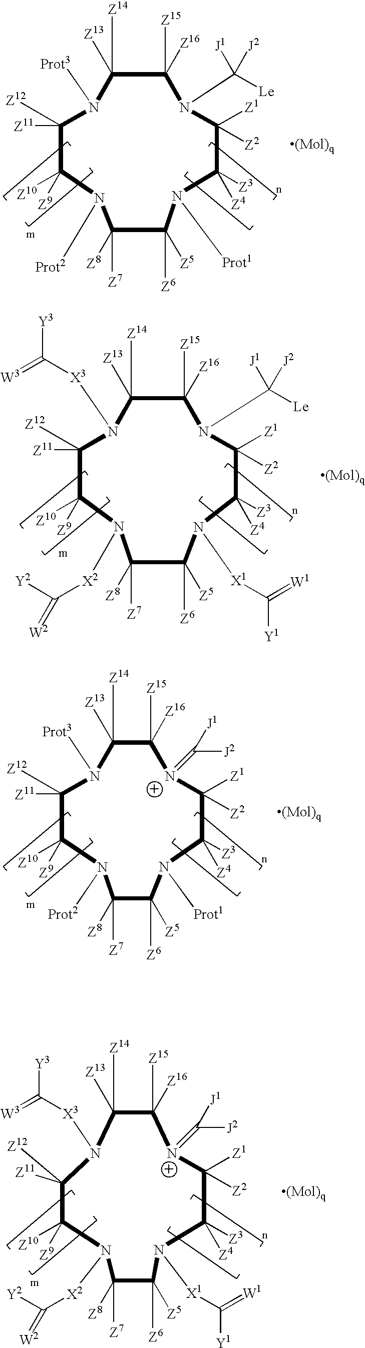

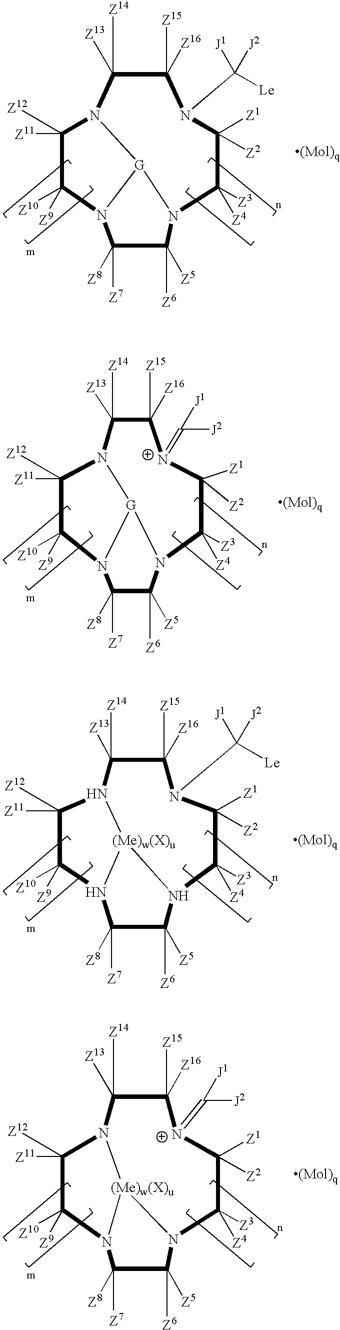

A is phosphorus or arsenic;
Z¹⁻¹⁶ is independently radical of hydrogen; chlorine; bromine; fluorine; iodine; nitro or nitrosogroup; sulphogroup; substituted or unsubstituted aliphatic or alicyclic or cyclic alkyl with or without one or more double or triple bonds and with or without heteroatoms; substituted or unsubstituted aromatic radical or its aryloxyderivate; hydroxyle; alcoxyle; S-substituted or S-unsubstituted thiole; substituted or unsubstituted amine; Z¹⁻¹⁶ also can constitute independently carbonyle and general functional derivates of carbonyle as oxime, hydrazone etc. but especially N-substituted or unsubstituted carboimidyle; thiocarbonyle; condensed substituted or unsubstituted benzoderivate;
n, m is independently 1 or 2;
X¹⁻³ is independently methylene or ethylene substituted as Z¹⁻¹⁶ especially with or without heteroatoms and multiple bonds; carbonyle; N-substituted or unsubstituted carboimidyle; thiocarbonyle;
Y¹⁻³ is independently methyl substituted as Z¹⁻¹⁶; hydroxyle; O-substituted hydroxyle with Z¹⁻¹⁶; S-substituted thiole; substituted or unsubstituted amine; hydroxylate or thiolate of metal cations or organic cations (for example: Na, Li, K, Rb, Cs, Ca, Mg, Al, Zn, Mn, Cr, Mo, ⁶⁴Cu, ⁶⁷Cu, ⁶⁷Ga, ⁹⁰Y, ¹¹¹In, ¹⁶³Sm, ¹⁶⁶Ho, ¹⁷⁷Lu, ²⁰¹Tl, ²¹²Bi, ammonium, primary, secondary, tertiary and quarternary alkyl and arylammonium, sulphonium and phosphonium salts and their combinations); Y¹⁻³ can constitute independently substituted hydroxylamine of formula:

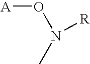

wherein A is independently methyl substituted as Z¹⁻¹⁶; metal cation or organic cation (for example: Na, Li, K, Rb, Cs, Ca, Mg, Al, Zn, Mn, Cr, Mo, ⁶⁴Cu, ⁶⁷Cu, ⁶⁷Ga, ⁹⁰In, ¹⁵³Sm, ¹⁶⁶Ho, ¹⁷⁷Lu, ²⁰¹Ti, ²¹²Bi, ammonium, primary, secondary, tertiary and quarternary alkyl and arylammonium, sulphonium and phosphonium salts and their combinations);
R is independently radical of hydrogen; substituted or unsubstituted aliphatic or alicyclic or cyclic alkyl with or without one or more double or triple bonds and with or without heteroatoms; substituted or unsubstituted aromatic radical;
R¹⁻² is independently hydrogen; halogene; substituted or unsubstituted aliphatic or alicyclic or cyclic alkyl with or without one or more double or triple bonds and with or without heteroatoms; substituted or unsubstituted aromatic radical or its aryloxyderivate; hydroxyle; alcoxyle; thiole; thioalcoxyle; substituted or unsubstituted amine; trialkylsilyl; trialkylsilyloxy, triarylsilyl; triarylsilyloxy; hydroxylate or thiolate of metal cations or organic cations (for example: Na, Li, K, Rb, Cs, Ca, Mg, Al, Zn, Mn, Cr, Mo, ⁶⁴Cu, ⁶⁷Cu, ⁶⁷Ga, ⁹⁰Y, ¹¹¹In, ¹⁵³Sm, ¹⁶⁸Ho, ¹⁷⁷Lu, ²⁰¹Tl, ²¹²Bi, ammonium, primary, secondary, tertiary and quarternary alkyl and arylammonium, sulphonium and phosphonium salts and their combinations);
W¹⁻³ is independently oxygen, sulphur, N-substituted or unsubstituted imidyl;
G is CZ¹⁻¹⁶ (include C⁺ as carbocation with Z¹⁻¹⁶ as anion), SiZ¹⁻¹⁶, SnZ¹⁻¹⁶, B, Al, P, As, PO, AsO, PS, ASS, AsZ¹⁻¹⁶, VZ¹⁻¹⁶, PZ¹⁻¹⁶;
Me is metal (or ion), especially: Cu, Ni, Fe, Zn, Cr, Mo, V; X is ligand for example Cl, Br, OH, etc.; u is from 1 to 15; w is 1 or 2 or 3 or ½ or ⅔ or 3/2), or formates TiOZ¹⁻¹⁶, TiNZ¹⁻¹⁶, MoP, MoN;
J¹⁻² is group (substituent, fragment) of the same type as Z¹⁻¹⁶;
Le is leaving group, especially of structure: —OR, —OH, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, —W¹⁻³H, —W¹⁻³R, N-benztriazolyl, 1-imidazolyl, succinimidyloxy, N-succinimidyl, N-phthalimidyl, N-phthalimidyloxy wherein Rⁱ⁻ⁱⁱⁱ are groups of the same type as R;
Prot¹⁻³ is independently protective group (or electron pair with negative charge) especially of general structure: —CHO, —COR, —COOR, —CONRⁱRⁱⁱ, —SO₂R, —SR, —R, —SiRⁱRⁱⁱRⁱⁱⁱ, —PORⁱRⁱⁱ, —PSRⁱRⁱⁱ, —PO(ORⁱ)(ORⁱⁱ); protective groups Prot¹ and Prot² or Prot¹ and Prot³ may be also connected to each other especially according to following general structure: —CRⁱRⁱⁱ—, —CO—, —COCO—, —CS—, —C(═NR)—, —COCRⁱRⁱⁱCO—, —CO—R—CO—, [—CRⁱRⁱⁱCRⁱRⁱⁱ]₂SO, [—CRⁱRⁱCRⁱRⁱⁱ]₂SO₂, [—CRⁱRⁱⁱCRⁱRⁱⁱ]₂P(O)OR, [—CRⁱRⁱⁱCRⁱRⁱⁱ]₂NR, —PO(OR)—, —SiRⁱRⁱⁱ—, —SnRⁱRⁱⁱ— wherein Rⁱ⁻ⁱⁱⁱ are groups of the same type as R. Prot¹⁻³ is for example methanesulphonyl, 4-toluenesulphonyl, trifluoromethanesulphonyl, nitrobenzenesulphonyl, benzenesulphonyl, naphthalenesulphonyl, formyl, acetyl, benzoyl, phthaloyl, trifluoroacetyl, tert.-butoxycarbonyl (Boc), 9H-fluoren-9-yl-methoxycarbonyl (Fmoc), benzyloxycarbonyl (Z), ethoxycarbonyl (Eoc), methoxycarbonyl (Meoc), methoxybenzylcarbonyl (Moz), trityl, benzyl, benzhydryl, 4,4′-dimethoxytrityl, 4-methoxybenzoyl, ethandioyl, propandioyl, carbonyl, thiocarbonyl etc.
Mol is protogenic acid (for example: mineral acid, substituted or unsubstituted carboxylic, sulphonic, phosphonic and phosphinic acid) or protophilic base (for example: pyridine, tetrahydrofurane, triethylphosphine) or Lewis acid (for example: BF₃, ZnCl₂, AlCl₃, FeBr₃) or neutral molecule bonded as e.g. in molecular cluster or associate (e.g. chloroform, toluene, cyclodextrine, calix[8]arene, polyethyleneglycole 800), q is from 0 to 10 or ½ or ⅔ or ¾, 4/3, 3/2;
by usage of one or more of these synthetic methods or synthetic routes anywhere in all synthetic approach in anyone from all used steps for synthesis of triprotected intermediates as described in xviii.
   a) by reaction of protected or unprotected macrocyclic tetramine or its salt or its appropriate anion (structure b1 and b2) with reactive intermediates of the type: Subst-[C(J¹)(J²)]-Le, (J¹)(J²)C═W¹⁻³, (J¹)(J²)C═W¹⁻³ and HW¹⁻³R mixture, according to schemes B1-B3:

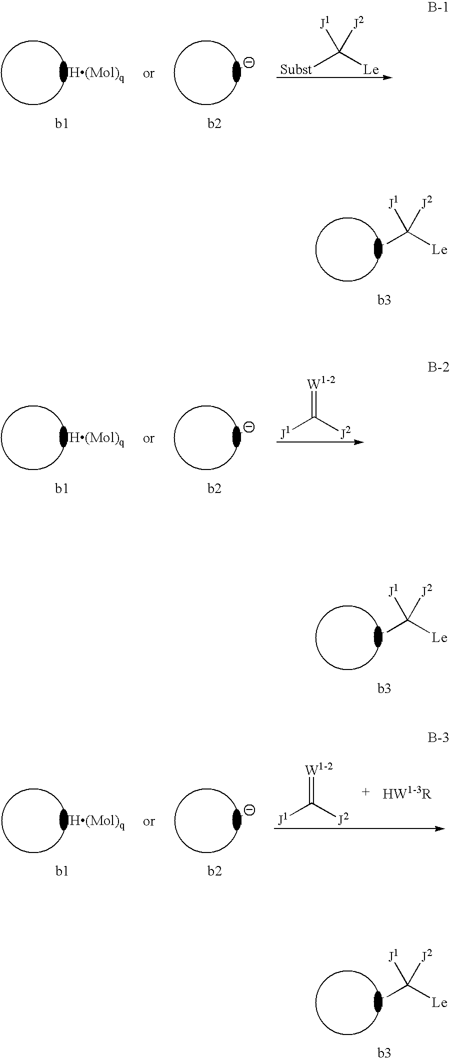

wherein Subst is general leaving group, of structure: —OR, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱⁱ)(SO₂Rⁱⁱ), —NSO₂R, -halogene, —NRⁱNRⁱⁱRⁱⁱⁱ, —SR, —SO₃H or —SO₂Y¹⁻³, wherein Rⁱ⁻ⁱⁱⁱ are groups of the same type as R. (Subst is for example: hydroxyle, alcoxyl, aryloxyl, alkyl amine, N,N-dialkylamine, N-alkylamine, arylsulfonyloxy, tosyloxy, mesyloxy, triflyloxy, acetoxy, benzoyloxy, N-benztriazolyl, trialkylsilyloxy, hydrazine and N-substituted hydrazine, benzyloxycarbonyloxy, tert.-butyloxycarbonyloxy, 1-imidazolyl, succinimidyloxy, N-succinimidyl, N-phthalimidyl, N-phthalimidyloxy, arylthio, thiole, S-alkylthiole etc.),
wherein symbol

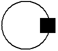

is chain 1 or chain 2 or chain 3 or chain 4 of structure:

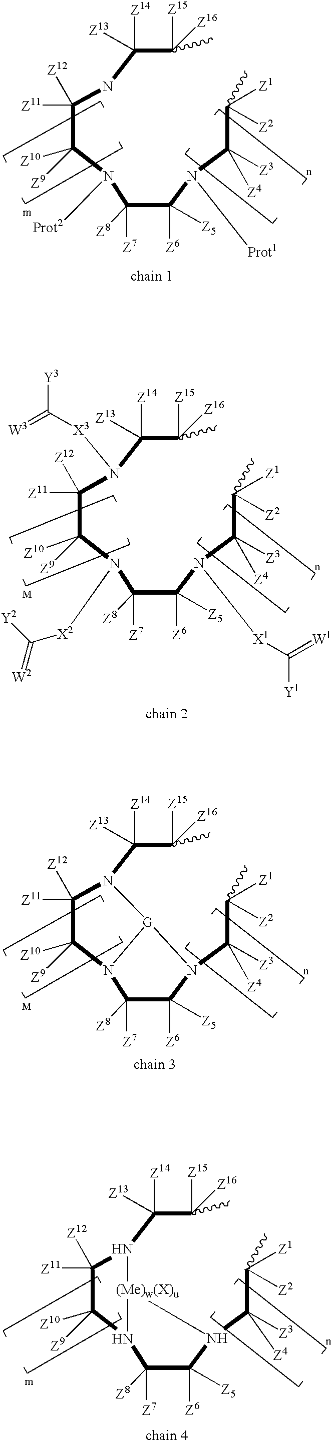

under conditions of general nucleofilic substitution: especially under conditions of phase-transfer catalysis, in aprotic polar solvents or its mixtures (as dimethylformamide or dimethylacetamide acetonitrile, dimethylsulphoxide or sulpholane or hexamethylphosphortriamide), in micellar medium, in solidphase (for example bonded N₄G⁻ on anex), with or without microwave irradiation, with or without ultrasonic irradiation, under conditions of high pressure (for example in autoclave), in aqueous phase in presence of pH-buffer, in milieu of water-free solvents with or without presence of base (for example: amines, aldimines, anex, carbonates, fluorides, thioethers), enzymatic catalysis, in presence of dehydrating agent or agent reacting with protogenic product reaction or in presence of Lewis acid as catalysators (for example ZnCl₂, BF₃.Et₂O, SiCl₄) etc.
or
b) by reaction of intermediate b3 with an agent eliminating Le⁻ anion by scheme B4.

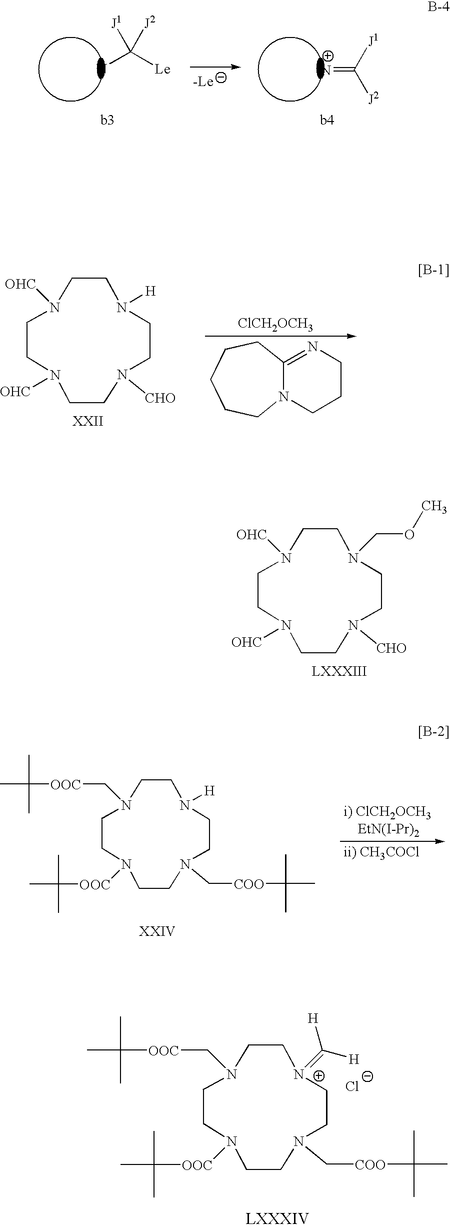

Intermediates used in previous classes i-xvii have great importance. Also in light of presented invention derivatives with active methylene have particular importance. N-alcoxymethyl derivatives are ones of the cardinal group. Preparation of unitriprotected N-methoxymethyl-triformylcyclene LXXIII (reaction B-1) can be carried out e.g. from chlorodimethylether as active methylene source. Yield of pure separated product is 91%. However, reaction with other sources of active methylene (e.g. N-benztriazolyl, sulfomethyl) is very important alternative for preparation of structures of this invention. In dependence on lipophility of both substrates reaction is carried out in large palette of solvent systems. In a lot of cases phase transfer catalysis is acceptable. N-methoxymethylation of XXIV by chlorodimethylether needs presence of proton acceptor. The best results gives so called proton sponges (DABCO in case of B-1; N-ethyl-N,N-diisopropylamine in case of B-2). Methyleniminium salt LXXXIV is obtained by action of acetyl chloride to methoxymethyl derivative of XIV. This intermediate disposes superb reactivity to nucleophiles.

Further, the invention relates to a pharmaceutical composition comprising a compound, a metal complex or a conjugate as described above together with pharmaceutically acceptable carriers, diluents or adjuvants. The composition may be suitable for diagnostic applications such as radioimaging or magnetic resonance imaging. On the other hand, the composition may be suitable for therapeutic applications such as radiotherapy or neutron capture therapy.

Finally, the present invention relates to a method of administering to a subject in need thereof a diagnostically or therapeutically effective amount of a compound, a metal complex or a conjugate as described above together with pharmaceutically acceptably carriers, diluents or adjuvants.

The present invention therefore relates to a process of production and synthesis of new selective and specific ligands usable as immunoradiopharmaceuticals, radiopharmaceuticals, supercancerostatics, targeted cancerostatics and general pharmaceuticals for cancer therapy and diagnostics. The invention also relates to a process of production and synthesis of new selective and specific ligands usable as general diagnostics and radiodiagnostics for general diagnostics methods in human or animals' therapy, medicinal science, biochemistry, and clinical analysis etc.

The present invention further relates to a process of preparation of ligands bondable on biological active substrates, e.g. monoclonal antibodies. The invention also relates to methods of preparation of specific and selective ligands by which new diagnosticals and radiodiagnosticals for general diagnostics methods in human or animals' therapy, medicinal science, biochemistry and clinical analysis etc. can be produced and synthesized.

Moreover, the present invention relates to methods for preparation of intermediates usable in selective or specific macrocyclic polyaza derivatives ligands preparation, synthesis and manufacturing. The inventors have found that the current synthetic methods for preparation of some structural fragments or all sceletons of our new selective and specific ligands are not suitable for large-scale production.

The invention is illustrated further by reference to the following non-limiting examples.

Products were characterized and identified by NMR (¹H NMR, ¹³C NMR, ³¹P NMR and IR), MS spectroscopy, elementar analysis and volumetric or HPLC analysis in some cases.

### EXAMPLE 1

### Preparation of tri-tert.-butyl ester 10-methoxymethyl-1,4,7,10-tetraazacyclododecane-1,4,7-triacetic acid (Bu₃DO3A-MOM) via tri-tert.-butyl ester 1,4,7,10-tetraazacyclododecane-1,4,7-triacetic acid (Bu₃DO3A)

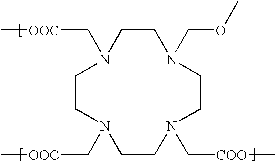

Into a nitrogen purged 25 l four necked round reaction vessel equipped with an addition funnel, temperature probe, nitrogen inlet adapter, and stirrer apparatus, there were placed bromide-free 0.643 kg (1.25 mol) of tri-tert.-butyl ester 1,4,7,10-tetraazacyclododecane-1,4,7-triacetic acid (Bu₃DO3A) base and 5 l of anhydrous methanol (water content up to 0.1%). To the solution was slowly added the solution prepared before of 37.54 grams (1.25 mol) of dry paraformaldehyde in 10 liters of anhydrous methanol (water content up 0.1%) in presence of no more than 0.5 g of potassium methoxide. The mixture was left to stand 80 hours at room temperature with slow (30 rpm) stirring. The HPLC (silica-C18/acetonitrile-methanol) probe indicates no input ester. After vacuum evaporation at low temperature (40-50° C.) crude product was eluted on AzaDVBP (AZacycles, Czech Republic) column by tert.-butylacetate-methyl-tert.-butylether mixture (2:1/vol:vol), then there were obtained 665 g of HPLC high pure (99.6%) Bu₃DO3A-MOM as viscous oily product.

### EXAMPLE 2

### Preparation of tri-tert.-butyl ester 10-benzyloxymethyl-1,4,7,10-tetraazacyclododecane-1,4,7-triacetic acid (Bu₃DO3A-BzOM) via tri-tert.-butyl ester 1,4,7,10-tetraazacyclododecane-1,4,7-triacetic acid (Bu₃DO3A)

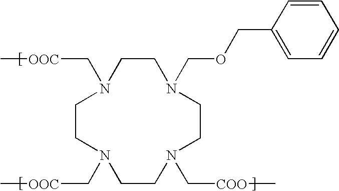

Into a 3 l three necked round bottom flask equipped with an addition funnel, temperature probe, reflux condenser with argon overpressure inlet adapter, on magnetic stirrer apparatus, there were placed bromide-free 121.5 g (0.236 mol) of tri-tert.-butyl ester 1,4,7,10-tetraazacyclododecane-1,4,7-triacetic acid (Bu₃DO3A) base and 1.75 l of dried benzene. At room temperature, to the solution was slowly added the solution of freshly distilled 37 grams (0.236 mol) chloromethylbenzylether in 250 ml of benzene. After addition the mixture was stirred at room temperature 45 minutes and then has been refluxed under argon for another 12 hours. After vacuum evaporation at low temperature (40-50° C.) crude product was eluted on AzaDVBP (Azacycles, Czech Republic) column by gradient elution with tert.-butylacetate—methyl-tert.-butylether mixture (from 1:1 to 1:3/vol:vol), then there were obtained 138 g of HPLC high pure (99.3%) Bu₃DO3A-BzOM as oily product.

### EXAMPLE 3

### Preparation of tri-tert.-butyl-ester 10-benzyloxymethyl-1,4,7,10-tetraazacyclododecane-1,4,7-triacetic acid (Bu₃DO3A-OcOM) via tri-tert.-butyl ester 1,4,7,10-tetraazacyclododecane-1,4,7-triacetic add (Bu₃DO3A)

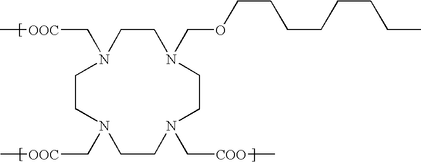

Into 250 ml three necked sulfonation bottle equipped with an addition funnel, temperature probe, reflux condenser with argon overpressure inlet adapter, on magnetic stirrer apparatus, there were placed bromide-free 5.15 g (0.01 mol) of tri-tert.-butyl ester 1,4,7,10-tetraazacyclododecane-1,4,7-triacetic acid (Bu₃DO3A) base and 100 ml of tert.-butanol (dried over fresh calcium hydride and redistilled). To the solution was at room temperature added solution of freshly distilled 1.79 grams (0.01 mol) chloromethyloctylether over 30 minutes. After addition of all ether the mixture was stirred under argon for 3 more hours. After vacuum evaporation at low temperature (40-50° C.) was crude product eluted on AzaDVBP (Azacycles, Czech Republic) column by gradient elution with tert.-butylacetate-methyl-tert.-butylether mixture (from 3:1 to 1:3/vol:vol), then there were obtained 6.23 g of HPLC high pure (99.5%) Bu₃DO3A-OcOM as oil-like product.

### EXAMPLE 4

### Preparation of tri-tert.-butyl-ester 10-methoxymethyl-1,4,7,10-tetraazacyclododecane-1,4,7-triacetic acid (Bu₃DO3A-MOM) via N-methoxymethylation tri-tert.-butyl ester 1,4,7,10-tetraazacyclododecane-1,4,7-triacetic acid (Bu₃DO3A)

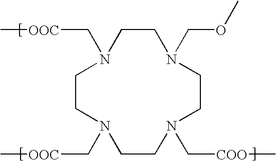

Into a very well sealing apparatus from 4 l three necked sulfonation bottle equipped with an addition funnel, temperature probe, argon overpressure inlet adapter, and magnetic stirrer, there were placed bromide-free 321.5 g (0.625 mol) of tri-tert.-butyl ester 1,4,7,10-tetraazacyclododecane-1,4,7-triacetic acid (Bu₃DO3A) base and 2.6 l of well dried acetonitrile. To the solution was slowly added 50.32 grams (0.625 mol) chloromethylmethylether over the period of 6 hours at argon atmosphere of overpressure. The mixture was stirred next 10 hours at room temperature. Potentiometric determination of chloride anions from reaction sample detected full conversion of chloroether. After vacuum evaporation at low temperature (30-40° C.) was crude product eluted on AzaDVBP (Azacycles, Czech Republic) column by tear.-butylacetate-methyl-tert.-butylether mixture (1:1/vol:vol), then there were obtained 332 g of HPLC high pure (99.5%) Bu₃DO3A-MOM as oily product.

### EXAMPLE 5

### Preparation of tri-tert.-butyl ester 10-benztriazolylmethyl-1,4,7,10-tetraazacyclododecane-1,4,7-triacetic acid (Bu₃DO3A-benztriazolylmethyl) via ester 1,4,7,10-tetraazacyclododecane-1,4,7-triacetic acid (Bu₃DO3A)

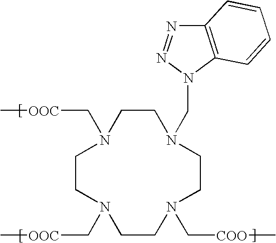

Into an apparatus from 8 l four necked reaction vessel equipped with an addition funnel, temperature probe, argon inlet adapter, and reflux condenser with efficient stirrer, there were placed bromide-free 220 g (0.393 mol) of (Bu₃DO3A) base and 51 of well dried acetonitrile. To the solution was slowly added solution of 64.4 grams (0.432 mol) N-hydroxymethylbenzotriazol in 1.5 l of acetonitrile over period of 1.5 hours at argon atmosphere. The mixture was stirred next 30 hours at reflux temperature. After evaporation in vacuo at low temperature (30-40° C.) was crude product eluted on AzaDVBP (Azacycles, Czech Republic) column by tert.-butylacetate-methyl-tert.-butylether mixture (1:1/vol:vol), then there were obtained 203 g of HPLC high pure (99.5%) Bu₃DO3A-benztriazolylmethyl.

### EXAMPLE 6

### Preparation of 10-methoxymethyl-1,4,7,10-tetraazacyclododecane-1,4,7-tricarboxylic acid tri-tert.-butyl ester (Boc3CyclenMOM) via N-methoxymethylation

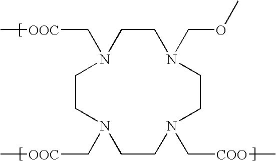

Into a nitrogen purged 10 l four necked round reaction vessel equipped with an addition funnel, temperature probe, nitrogen inlet adapter, and stirrer apparatus, there were placed chloride-free 0.266 kg (0.563 mol) of N′,N″,N′″-tris(tert.-butoxycarbonylmethyl)cyclene base, 69 g (0.6 mol) diisopropylmethylamine and 4.2 l of freshly dried and distilled acetonitrile. To the solution was slowly added solution of 45.3 grams (0.563 mol) chloromethylmethylether in 0.4 liters of acetonitrile of the same quality. The mixture was left to stand 80 hours at room temperature with slow (30 rpm) stirring. The HPLC (silica-C18/acetonitrile-methanol) probe does not indicate input ester. After vacuum evaporation at low temperature (40-50° C.) was crude product eluated on AzaDVBP (Azacycles, Czech Republic) column by tert.-butylacetate-methyl-tert.-butylether mixture (2:1/vol:vol), then there were obtained 273 g of HPLC high pure (99.6%) Boc3 CyclenMOM as viscous oil-like product.

### EXAMPLE 7

### Preparation of 10-benzyloxymethyl-1,4,7,10-tetraazacyclododecane-1,4,7-tricarboxylic acid tri-tert.-butyl ester (Boc3Cyclen-BzOM) via N-benzyloxymethylation

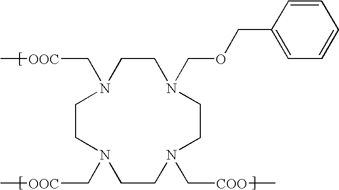

Into a nitrogen purged 1 l three necked round reaction vessel equipped with an addition funnel, temperature probe, nitrogen inlet adapter, and magnetic stirrer apparatus there were placed chloride-free 84 g (0.177 mol) of N′,N″,N′″-tris(tert.-butoxycarbonylmethyl)cyclene base, 34.58 g (0.177 mol) dicyclohexylmethylamine and 390 ml of freshly dried and distilled dioxane. To the solution was slowly added solution of 30.4 grams (0.194 mol) chloromethylbenzylether (min. 84% quality) in 150 ml of dioxane of the same quality. The mixture was left to stand 5 hours at room temperature with slow (10 rpm) stirring. Obtained solution was filtered. Filtrate was concentrated under vacuum and triturated with 550 ml warm toluene. After hot filtration liquid phase was chromatographed on carboxylic catex column (eluated by tert.-Butylamine/tert.-Butanol (1:16)). At low temperature (40-45° C.) there was obtained crude oil-like viscous product after vacuum evaporation.

### EXAMPLE 8

### Preparation of tri-tert.-butyl ester 10-[3-(N-phthalimidyl)propyl-ethoxy-phosphinoylmethyl]-1,4,7,10-tetraazacyclododecane-1,4,7-triacetic acid via Bu₃DO3A-MOM

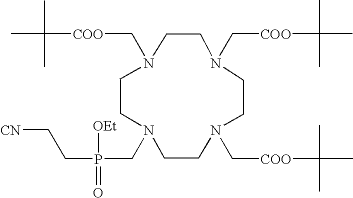

Into a 2 l three necked round bottom flask equipped with an addition funnel, temperature probe, reflux condenser with argon overpressure inlet adapter and magnetic stirrer apparatus were placed 120 g (0.214 mol) of Bu₃DO3A-MOM and 0.82 l of dried tetrahydrofurane/acetonitrile (1:1) mixture. To the solution was added at room temperature the solution of 32.4 grams (0.22 mol) ethyl (2-cyanoethyl)phosphinate in 250 ml of acetonitrile. After addition, the mixture has been refluxed under argon 16 hours. 121 g of high pure oily viscous product were obtained after vacuum evaporation at low temperature (40-50° C.). Total yield: 84 percent.

### EXAMPLE 9

### Preparation of tri-tert.-butyl ester 10-[ethoxy-(4-nitrobenzyl)phosphinoylmethyl]-1,4,7,10-tetraazacyclododecane-1,4,7-triacetic acid via Bu₃DO3A-BzOM

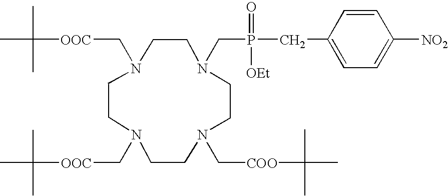

Into a 10 l four necked reaction vessel equipped with an addition funnel, reflux condenser with nitrogen overpressure inlet adapter and stirring apparatus were placed 254 g (0.4 mol) of Bu₃DO3A-BzOM and 3.2 l of dried benzene. To the solution was added at room temperature the solution of 101 grams (0.44 mol) ethyl (4-nitrobenzyl)phosphinate in 2000 ml of anhydrous benzene. After addition, the mixture has been refluxed under nitrogen overpressure 12 hours. 257 g of high pure product were obtained after vacuum evaporation at low temperature (40-50° C.) as highly viscous pale yellow oil. Total yield: 85 percent.

### EXAMPLE 10

### Preparation of tri-tert.-butyl ester 10-[(2-benzyloxycarbonylethyl)-ethoxy-phosphinoylmethyl]-1,4,7,10-tetraazacyclododecane-1,4,7-triacetic acid via Bu₃DO3A-benztriazolylmethyl

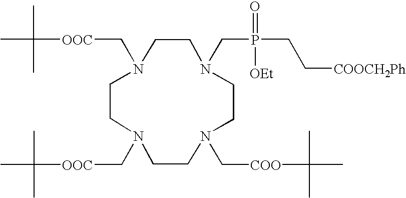

Into a 50 ml flask equipped with septum, a syringe, reflux condenser with argon overpressure inlet adapter, and magnetic stirrer apparatus, there were placed 330 mg (0.51 mmol) of Bu₃DO3A-Benztriazolylmethyl and 22 ml of dried acetonitrile. To the solution was added at reflux temperature solution of 144 mg (0.56 mmol) benzyl 3-(1-ethoxy-1-oxophosphoranyl)propanoate in 10 ml of acetonitrile. After addition, the mixture was refluxed under argon next 48 hours. 371 mg of high pure colorless oil-like viscous product were obtained after vacuum evaporation. Total yield: 93 percent.

### EXAMPLE 11

### Preparation of 2 dibenzylamino-ethyl)-7-formyl-1,4,7,10-tetraazacyclododec-1-ylmethyl)-phosphinic acid ethyl ester via 7-methylene-octahydro-2a,4a,9a-triaza-7-azoniacycloocta[cd]pentalene chloride

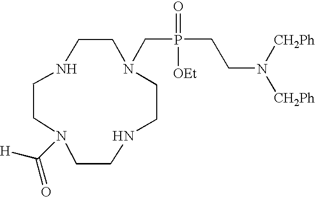

To a solution of 7-methylene-octahydro-2a,4a,9a-triaza-7-azoniacycloocta[cd]pentalene chloride (3.98 g; 17.24 mmol) in dry DMF (70 ml) was added solution of ethyl 2-(dibenzylamino)ethyl]phosphinate (5.47 g; 17.24 mmol) in dry dimethylformamide (50 ml) at room temperature. Mixture was stirred at 60-65° C. under nitrogen for 12 hours. The solvent was removed in vacuo, and the residue was partitioned between dichloromethane (200 ml) and sat. aq. NH₄Cl (100 ml). The organic phase was extracted with water (100 ml), brine (50 ml), dried over fresh mol. sieve and the solvent removed in vacuo. After trituration in isopropanol there have been given pure 6.57 g of an oil-like pale yellow product. Total yield: 72 percent.

### EXAMPLE 12

### Preparation of tri-tert.-butyl ester 10-[3-(N-Phthalimidyl)propyl-ethoxy-phosphinoylmethyl]-1,4,7,10-tetraazacyclododecane-1,4,7-triacetic acid via Bu₃DO3A-MOM

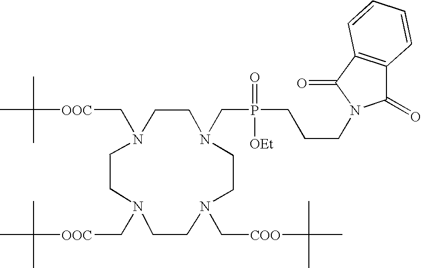

Tri-tert.-butyl ester 10-[3-(N-phthalimidyl)propyl-ethoxy-phosphinoylmethyl]-1,4,7,10-tetraazacyclododecane-1,4,7-triacetic acid was prepared from Bu3DO3A-MOM by same method as it has been described in Example 8 with 3-(N-phthalimidyl)propylphosphinic acid ethyl ester on place of ethyl [3-(N-phthalimidyl)propyl]phosphinate. Yield was 88 percent.

### EXAMPLE 13

### a) Preparation of 10-methoxymethyl-triformylcyclene via triformylcyclene

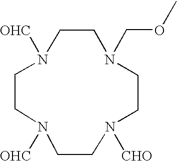

In a very well sealing apparatus from 1000 ml three necked sulfonation bottle equipped with an addition funnel, temperature probe, argon overpressure inlet adapter, and magnetic stirrer, there were placed 40 g (0.156 mol) of triformylcyclene, 162 g ethyldiisopropylamine and 600 ml of well dried acetonitrile. To the solution there were slowly added 12.56 grams (0.156 mol) chloromethylmethylether over period of 6 hours at argon atmosphere of overpressure. The mixture was stirred next 10 hours at room temperature yet. Potentiometric determination of chloride anions from reaction sample detected full conversion of chloroether. After vacuum evaporation at low temperature (30-40° C.) was crude product twice eluted on 5 l AzaDVBP (Azacycles, Czech Republic) column by tert.-butylacetate-methyl-tert.-butylether mixture (1:1/vol:vol), then there were obtained 42 g of HPLC high pure (98.2%) oily product.

### b) Preparation of 4,7,10-triformyl-1-methylene-4,7,10-triaza-1-azoniacyclododecane chloride via 10-methoxymethyl-triformylcyclene

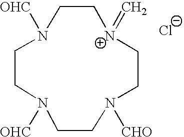

A mixture of 15.02 g (50 mmol) 10-methoxymethyl-triformylcyclene (see Example 13a) and 30 ml dried acetonitrile was added dropwise to a solution of methyltrichlorosilane 7.5 g (50 mmol) in dried acetonitrile (20 ml) at room temperature. The reaction is exothermic and a cold water bath was used to avoid a rise of temperature. The mixture was allowed to stir for five minutes more at room temperature to complete the reaction, concentrated in vacuo leading to a white salt. This salt was washed with anhydrous ether and transferred to vacuum dessicator for storage. Thus, 4,7,10-triformyl-1-methylene-4,7,10-triaza-1-azoniacyclododecane chloride was obtained in yield 97 percent.

### c) Preparation of triformylcyclam and 11-methoxymethyl-triformylcyclam via cyclam

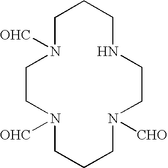

A mixture of cyclam 3 g (15 mmol) and chloral hydrate 12.4 g, (75 mmol) was dissolved in 100 ml of ethanol in a round bottom bottle. The solution was stirred at 65° C. as long as input cyclame absence was indicated by HPLC analysis (silica-DEAE/ammonium acetate-ammonia-methanol). The reaction mixture was concentrated under vacuum to dryness. Total yield was 4 g (94%) of colorless crystalline product (after flash chromatography on silica-DEAE in DCM:EtOH:NH₃ mixture).

### d) Preparation of 11-methoxymethyl-triformylcyclam via triformylcyclam

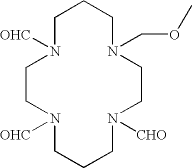

11-methoxymethyl-triformylcyclam was prepared from triformylcyclam by same method as it has been described in Example 13a. Yield was 87 percent.

### EXAMPLE 14

### Preparation of [2-4-nitrophenyl ethyl]-(4,7,10-triformyl-4,7,10-tetraazacyclododec-1-ylmethyl)-phosphinic acid ethyl ester via 4,7,10-triformyl-1-methylene-4,7,10-triaza-1-azoniacyclododecane chloride

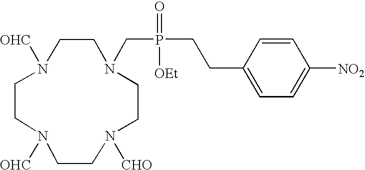

To a solution of 4,7,10-Triformyl-1-methylene-4,7,10-triaza-1-azoniacyclododecane chloride 4 g (13.1 mmol; see Example 13a) in dry DMF (40 ml) was added solution of [2-(4-nitrophenyl)-ethyl]phosphinic acid ethyl ester 3.19 g (13.1 mmol) in dry dimethylformamide (20 ml) at room temperature. Mixture was stirred at 65-70° C. under nitrogen for 10 hours. The solvent removed in vacuo to yield a yellow oil. The product was purified by alumina column chromatography (gradient elution from dichloromethane to 4% methanol-dichloromethane) giving a yellow oil. Yield of separated [2-(4-nitrophenyl)ethyl]-(4,7,10-triformyl-1,4,7,10-tetraazacyclododec-1-ylmethyl)-phosphinic acid ethyl ester was 68 percent.

### EXAMPLE 15

### Preparation of (4,7,10-triformyl-1,4,7,10-tetraazacyclododec-1-ylmethyl)phosphonic acid diethyl ester via 10-methoxymethyl-triformylcyclene

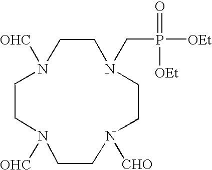

A mixture of 2.35 g (30 mmol) acetyl chloride and 10 ml od dried and freshly distilled (from P₄O₁₀) acetonitrile is added dropwise to a solution 9 g (30 mmol) 10-methoxymethyl-triformylcyclene (see Example 13a) and 30 ml dried acetonitrile. The reaction is exothermic and a cold water bath was used to avoid a rise of temperature. The mixture is allowed to stir for 30 minutes more at room temperature to complete the reaction. Solution of triethylphosphite 5 g (30 mmol) in dried acetonitrile (10 ml) at room temperature was added dropwise thereafter. The mixture was allowed to stir for 50 minutes more at room temperature to complete the reaction, concentrated in vacuo. Thus was obtained (4,7,10-triformyl-1,4,7,10-tetraazacyclododec-1-ylmethyl)phosphonic acid diethyl ester in almost quantitative yield as oil-like yellowish product.

### EXAMPLE 16

### Preparation of (4-Iodophenyl)-(4,7,10-triformyl-1,4,7,10-tetraazacyclododec-1-ylmethyl)-phosphinic acid ethyl ester via 10-methoxymethyl-triformylcyclene

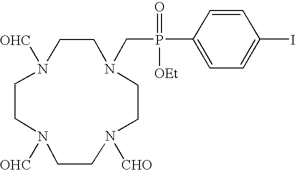

Into a 1 l three necked reaction vessel equipped with an addition funnel (with servo and pressure correction), electronic temperature meter bonded to thermostat, nitrogen overpressure inlet adapter and stirring apparatus there were placed 25 g (83.2 mmol) of 10-methoxymethyl-triformylcyclene (se Example 13a) and 360 ml of dried dimethylformamide. To the solution was added at room temperature solution of 24.6 grams (83.2 mmol) (4-Iodophenyl)phosphinic acid ethyl ester in 200 ml of dried dimethylformamide. After addition, the mixture has been stirred at 95° C. under nitrogen overpressure for 12 hours. After vacuum evaporation at low temperature (40-50° C.) was reaction mass treated by same procedure as described in Example 14. Total yield: 72 percent.

### EXAMPLE 17

### Preparation of N-[2-(diethoxyphosphoryl)ethyl]-1,4,7,10-tetraazacyclododecane-molybdenum-tricarbonyl complex and [2-(1,4,7,10-tetraazacyclododec-11 yl)-ethyl]-phosphonic acid via 1,4,7,10-tetraazacyclododecane-molybdenum-tricarbonyl complex

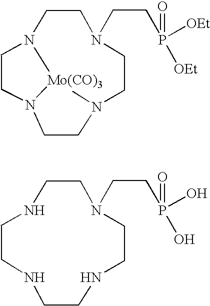

1,4,7,10-Tetraazacyclododecane 0.4 g (2.3 mmol) and molybdenum hexacarbonyl 0.6 g (2.3 mmol) in dry dibutyl ether (20 ml) were heated at reflux under argon for 2 h to give a bright yellow precipitate of the 1,4,7,10-tetraazacyclododecane-molybdenum-tricarbonyl complex which was filtered under argon and dried in vacuo.

Into a 25 ml four necked reaction bottle equipped with septum, a syringe, reflux condenser with argon overpressure inlet adapter and magnetic stirrer apparatus there were placed 70.4 mg (0.2 mmol) of 1,4,7,10-tetraazacyclododecane-molybdenum-tricarbonyl complex and 8 ml of well dried dimethylformamide. After the solution was complete, 2,5,6,7,8,9-hexahydro-3H-imidazo[1,2-a]azepine (28 mg) was added. To this solution was added very slowly 49 mg (0.2 mmol) (2-bromoethyl)phosphonic acid diethyl ester in 5 ml of dried acetonitrile at reflux temperature. After addition, the mixture was refluxed under argon next 5 hours. The solvent was removed in vacuo and the residue taken up in degassed 5 ml 20% aqueous hydrochloride acid. The resulting acidic mixture was oxidized in air until no more carbon monoxide evolved, and then it was washed with chloroform (3×10 ml). Aqueous phase was evaporated at vacuo. The residual oil was dissolved in 35% hydrochloric acid and refluxed over night and evaporated to dryness and separated by chromatography on alumina.

### EXAMPLE 18

### Preparation of diethyl 2-(9b-oxoperhydro-2a,4a,7,9a-tetraaza-9b□⁵-phosphacycloocta[ca]pentalen-7-yl)-2-(4-nitrophenyl)ethyl]phosphonate via perhydro-2a,4a,7,9a-tetraaza-9b□⁵-phosphacycloocta[cd]pentalen-9b-one (cyclen-phosphine oxide)

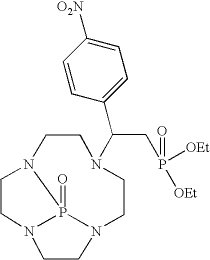

Into a 250 ml four necked reaction bottle equipped with an addition funnel, thermometer, reflux condenser with argon overpressure inlet adapter and magnetic stirrer apparatus, there were placed under argon 18 g (83.2 mmol) of vyclen-phosphine oxide and 120 ml of well dried and freshly distilled dioxane. To this solution was added very slowly 23.7 g (83.2 mmol) [2-(4-nitrophenyl)vinyl]phosphonic acid diethyl ester in 60 ml of dried dioxane over the period of 4 hours. After addition, the mixture was stirred under argon next 2 hours. Reaction mixture was refluxed about one hour thereafter. 41.6 g of product was obtained after evaporation of reaction mixture at high vacuo (0.01 torr). Yield is almost quantitative.

### EXAMPLE 19

### Preparation of diethyl [(4-nitrophenyl)(perhydro-2a,4a,7,9a-tetraaza-9b-boracycloocta[cd]pentalen-7-yl)methyl]phosphonate and [(4-nitrophenyl)-(1,4,7,10-tetraazacyclododec-1-yl)-methyl]-phosphonic acid via perhydro-2a,4a,7,9a-tetraaza-9b-boracycloocta[cd]pentalene (boron-cyclen)

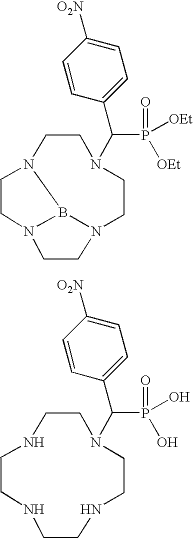

Into a 25 ml four necked reaction bottle equipped with an addition funnel, thermometer, reflux condenser with argon overpressure inlet adapter and magnetic stirrer apparatus, there were placed 1.5 g (8.33 mmol) of boron-cyclen and 8 ml of well dried and freshly distilled dioxane. After the solution is complete, 0.2 g of sodium hydride (60% suspension in paraffin oil) was added under argon atmosphere. Mixture was irradiated in ultrasonic bath (90 W, 20 kHz) about 40 minutes. To this solution there were added very slowly 3.23 g (9.16 mmol) [bromo(4-nitrophenyl)methyl]-phosphonic acid diethyl ester in 8 ml of dried dioxane over the period of 4 hours. After addition, the mixture was stirred and refluxed under argon next 2 hours. Thereafter, solvent was evaporated at vacuo. NMR analysis detected complete boron derivate consumption. To the residue was added 10 ml of ethanol—water (4:1) mixture. Thereafter, mixture was evaporated and the residue was dissolved in 15 ml of 35% hydrochloric acid. This mixture was refluxed about 6 hours under argon. After evaporation, residue was chromatographed on 2 l Dowex 50 (H⁺-form) column. Non-amine impurities were eluted with water, aminic compounds with aqueous ammonia. Eluents containing product were combined and evaporated at vacuo. After chromatography purification on Amberlite CG-50 (H⁺-form) column, there were obtained 2.6 g of product [(4-nitrophenyl)-(1,4,7,10-tetraazacyclododec-1-yl)-methyl]-phosphonic acid. Total yield is 81 percent.

### EXAMPLE 20

### Preparation of tert.-butyl 3-[[(9b-oxoperhydro-2a,4a,7,9a-tetraaza-9bλ⁵-phosphacycloocta[cd]pentalen-7-yl)(4-methoxyphenyl)methyl](ethoxy)phosphoryl]propanoate and 3-[(4-methoxyphenyl)-(1,4,7,10-tetraazacyclododecanyl)methyl]hydroxyphosphorylpropanoic acid via perhydro-2a,4a,7,9a-tetraaza-9b□⁵-phosphacycloocta[cd]pentalen-9b-one (cyclen-phosphine oxide)

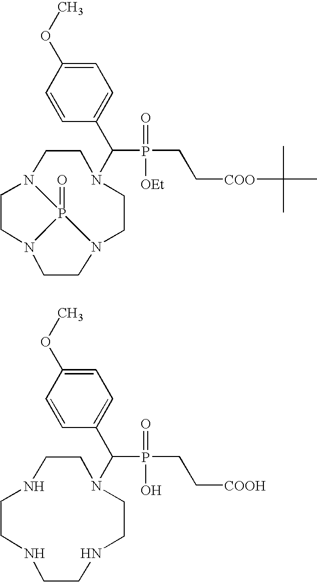

Into a 25 ml four necked reaction bottle equipped with an addition funnel, temperature meter, reflux condenser with argon overpressure inlet adapter and magnetic stirrer apparatus, there were placed 1.8 g (8.33 mmol) of cyclen-phosphine oxide and 8 ml of well dried and freshly distilled dioxane. After the solution was complete, 0.2 g of sodium hydride (60% suspension in paraffin oil) was added under argon atmosphere. Mixture was irradiated in ultrasonic bath (90 W, 20 kHz) about 40 minutes. To this solution there were added very slowly 3.23 g (9.16 mmol) 3-{[bromo-(4-methoxyphenyl)methyl]ethoxyphosphinoyl}-propionic acid tert.-butyl ester in 8 ml of dried dioxane over the period of 4 hours. After the addition, the mixture was stirred end refluxed under argon next 2 hours. Thereafter, solvent was evaporated at vacuo. NMR analysis detected complete boron derivate consumption. To the residue was added 10 ml of ethanol-water (4:1) mixture. Thereafter, mixture was evaporated and the residue was dissolved in 15 ml of 35% hydrochloric acid. This mixture was refluxed about 6 hours under argon. After evaporation, residue was chromatographed on 2 l Dowex 50 (H⁺-form) column. Non-amine impurities were eluted with water, aminic compounds with aqueous ammonia. Eluents containing product were combined and evaporated at vacuo. After chromatography purification on Amberlite CG-50 (H⁺-form) column there were obtained 2.8 g 3-[(4-methoxyphenyl)-(1,4,7,10-tetraazacyclododecanyl)methyl]hydroxyphosphorylpropanoic acid. Total yield is 78 percent.

### EXAMPLE 21

### Preparation of tri-tert.-butyl ester 10-[butyl-ethoxy-phosphinoylmethyl]-1,4,7,10-tetraazacylododecane-1,4,7-triacetic acid via of N′,N″,N′″-tris(tert.-butoxycarbonylmethyl)cyclene

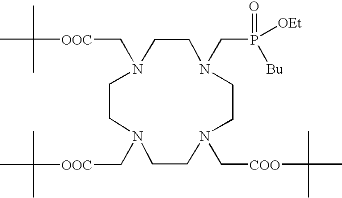

To a solution of N′,N″,N′″-tris(tert.-butoxycarbonylmethyl)cyclene base (4 g, 8.46 mmol) in dry acetonitrile (70 ml) there was added solution of (butyl-ethoxy-phosphinoylmethyl)-trimethyl-ammonium bromide (5.1 g, 16.92 mmol) in dry acetonitrile (50 ml) at room temperature. Mixture was refluxed under nitrogen for 3 hours. The solvent was removed in vacuo, and the residue was partitioned between dichloromethane (50 ml) and 10% aqueous NH₄Cl (15 ml). The organic phase was extracted with water (10 ml), dried over fresh mol. sieve and the solvent removed in vacuo. 4.9 g of pure tri-tert.-butyl ester 10-[butyl-ethoxy-phosphinoylmethyl]-1,4,7,10-tetraazacyclododecane-1,4,7-triacetic was obtained as oil-like product. Total yield: 85 percent.

### EXAMPLE 22

### Preparation of tri-tert.-butyl ester 10-[3-(N-phthalimidyl)ethyl-ethoxy-phosphinoylmethyl]-1,4,7,10-tetraazacyclododecane-1,4,7-triacetic acid via of N′,N″,N′″-tris(tert.-butoxycarbonylmethyl)cyclene

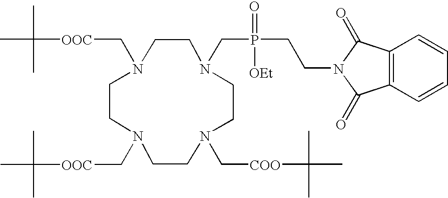

Into a 3 l four necked reaction vessel equipped with an addition funnel (with servo), electronic temperature meter, reflux condenser with nitrogen overpressure inlet adapter and a strong mechanic stirrer apparatus, there were placed 112 g (0.217 mol) of N′,N″,N′″-tris(tert.-butoxycarbonylmethyl)cyclene base and 1.55 liters of dried acetonitrile. After the solution was complete, temperature was raised to boiling under nitrogen very low overpressure atmosphere. To this solution was added through peristaltic pump 93.2 g (0.217 mol) trifluoromethanesulfonic acid [2-(N-phthalimidyl)ethyl]-ethoxy-phosphinoylmethyl ester in 420 ml of dried acetonitrile over the period of 14 hours. After addition, the mixture was stirred at continuous boiling under nitrogen for the next 2 hours. Temperature was decreased to 35° C. on end of reaction and saturated solution of 42.4 g methyldicyclohexylamine in dioxane was added quickly in one portion (strong pump) to reaction mass. Reaction mixture was carefully filtered at nitrogen overpressure thereafter. Crude solution of product was concentrated at vacuo and after trituration with dichloroethane—pentane mixture product was obtained as pure viscous pale yellow oil. 116 g of 10-[3-(N-phthalimidyl)ethyl-ethoxy-phosphinoylmethyl]-1,4,7,10-tetraazacyclododecane-1,4,7-triacetic acid were obtained after double trituration and recrystallization from toluene—ethyl acetate (1:3) mixture after two week's standing. Total yield: 67 percent.

### EXAMPLE 23

### Preparation of tri-tert.-butyl ester {10-[2-benzyloxycarbonyl-1-(4-methoxyphenyl)ethyl]-ethoxy-phosphinoylmethyl}-1,4,7,10-tetraazacyclododecane-1,4,7-triacetic acid via of N′,N″,N′″-tris(tert.-butoxycarbonylmethyl)cyclene

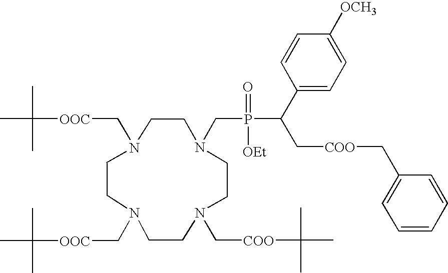

Tri-tert.-butyl ester {10-[2-benzyloxycarbonyl-1-(4-methoxyphenyl)ethyl]ethoxy-phosphinoylmethyl}-1,4,7,10-tetraazacyclododecane-1,4,7-triacetic acid was prepared from N′,N″,N′″-tris(tert.-butoxycarbonylmethyl)cyclene by same method as it has been described in Example 22 with 3-(ethoxy-trifluoromethanesulfonyloxymethyl-phosphinoyl)-3-(4-methoxyphenyl)-propionic acid benzyl ester on place of trifluoromethanesulfonic acid [2-(N-phthalimidyl)ethyl]-ethoxy-phosphinoylmethyl ester. Yield was 72 percent.

### EXAMPLE 24

### Preparation of tri-tert.-butyl ester {10-[2-benzyloxycarbonyl-1-(4-nitrophenyl)ethyl]-ethoxy-phosphinoylmethyl}-1,4,7,10-tetraazacyclododecane-1,4,7-triacetic acid via N′,N″,N′″-tris(tert.-butoxycarbonylmethyl)cyclene

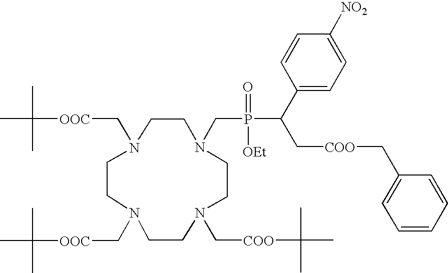

Tri-tert.-butyl ester {10-[2-benzyloxycarbonyl-1-(4-nitrophenyl)ethyl]-ethoxy-phosphinoylmethyl}-1,4,7,10-tetraazacyclododecane-1,4,7-triacetic acid was prepared from N′,N″,N′″-tris(tert.-butoxycarbonylmethyl)cyclene by same method as it has been described in Example 22 with 3-(ethoxy-trifluoromethanesulfonyloxymethyl-phosphinoyl)-3-(4-nitrophenyl)-propionic acid benzyl ester on place of trifluoromethanesulfonic acid [2-(N-phthalimidyl)ethyl]-ethoxy-phosphinoylmethyl ester. Yield was 84 percent.

### EXAMPLE 25

### Preparation of 10-[butyl-hydroxy-phosphinoylmethyl]-1,4,7,10-tetraazacyclododecane-1,4,7-triacetic acid via tri-tert.-butyl ester 10-[butyl-ethoxy-phosphinoylmethyl]-1,4,7,10-tetraazacyclododecane-1,4,7-triacetic acid

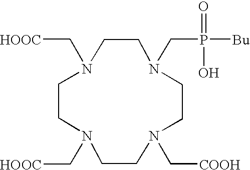

To a solution of tri-tert.-butyl ester 10-[butyl-ethoxy-phosphinoylmethyl]-1,4,7,10-tetraazacyclododecane-1,4,7-triacetic acid (8.2 g, 12.11 mmol; see Example 21) in dry dioxane (50 ml) was added tricaprylmethylammonium chloride (180 mg). Thereafter there have been added 6 ml of concentrated hydrochloric acid and 5 ml of water. Mixture was irradiated in microwave oven for 25 seconds at 700 W. The solvent was carefully removed in high vacuo at low temperature (30-40° C.), and the residue was triturated by dichloromethane and filtered. After avaporation, residue was chromatographed on 2 l Dowex 50 (H⁺-form) column. Non-amine impurities were eluted with water, aminic compounds with aqueous ammonia. Eluents containing product were combined and evaporated at vacuo. After chromatography purification on Amberlite CG-50 (H⁺-form) column there have been obtained 5.23 g of product as free acid. Total yield: 90 percent.

### EXAMPLE 26

### Preparation of 10-[2,2-bis-(diethoxyphosphoryl)ethyl]-1,4,7,10-tetraazacyclododecane-1,4,7-tricarboxylic acid tri-tert.-butyl ester via 1,4,7,10-tetraazacyclododecane-1,4,7-tricarboxylic acid tri-tert.-butyl ester

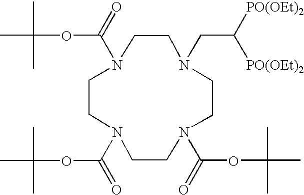

Into a 50 ml flask equipped with septum, a syringe, reflux condenser with argon overpressure inlet adapter and magnetic stirrer apparatus, there were placed 305 mg (0.645 mmol) of 1,4,7,10-tetraazacyclododecane-1,4,7-tricarboxylic acid tri-tert.-butyl ester and 12 ml of well dried acetonitrile. To this solution there were added very slowly 213 mg (0.71 mmol) tetraethyl ethylidene bisphosphonate in 10 ml of dried acetonitrile at reflux temperature over the period of 6 hours. After addition, the mixture was refluxed under argon next 12 hours. 433.6 mg of high pure 10-[2,2-bis-(diethoxyphosphoryl)ethyl]-1,4,7,10-tetraazacyclododecane-1,4,7-tricarboxylic acid tri-tert.-butyl ester were obtained after ultra high vacuum evaporation and purification on silica column (eluent: ethyl acetate/hexane/tert.-butanol mixture). Total yield: 87 percent.

### EXAMPLE 27

### Preparation of tri-tert.-butyl ester 10-[2-(diethoxy-phosphoryl)-ethyl]-1,4,7,10-tetraazacyclododecane-1,4,7-triacetic acid via N′,N″,N′″-tris(tert.-butoxycarbonylmethyl)cyclene

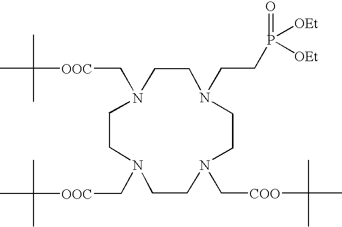

Into a 50 ml flask equipped with septum, a syringe, reflux condenser with argon overpressure inlet adapter and magnetic stirrer apparatus, here were placed 618 mg (1.2 mmol) of N′,N″,N′″-tris(tert.-butoxycarbonylmethyl)cyclene and 20 ml of well dried acetonitrile. To this solution there were added very slowly 217 mg (1.32 mmol) vinyl-phosphonic acid diethyl ester in 10 ml of dried acetonitrile at reflux temperature over the period of 6 hours. After addition, the mixture was refluxed under argon next 15 hours. 603 mg of high pure tri-tert-butyl ester 10-[2-(diethoxy-phosphoryl)-ethyl]-1,4,7,10-tetraazacyclododecane-1,4,7-triacetic acid was obtained after ultra high vacuum evaporation and purification on silica column (eluent: ethyl acetate/hexane/tert.-butanol mixture). Total yield: 74 percent.

### EXAMPLE 28

### Preparation of [2-(N-phthalimidyl)ethyl]-(4,7,10-triformyl-1,4,7,10-tetraazacyclododec-1-ylmethyl)-phosphinic acid ethyl ester and [2-(N-phthalimidyl)ethyl]-(1,4,7,10-tetraazacyclododec-1-ylmethyl)phosphinic acid ethyl ester via 10-benzyloxymethyl-triformylcyclene

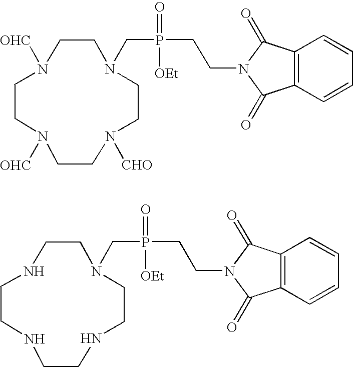

To a solution of 10-benzyloxymethyl-triformylcyclene (see Example 1 or Example 4) (1.84 g, 4.88 mmol), 2-(N-phthalimidyl)ethyl]phosphinic acid ethyl ester (1.3 g, 4.88 mmol) in dry dimethylformamide (25 ml) there were added 35 g montmorillonite and suspension was evaporated carefully to dryness. Immobilized reactants were irradiated in microwave oven for 55-60 seconds at 700 W (PTFE flask). The solid phase was triturated by dichloromethane, next by dimethylformamide (3×150 ml) and filtered. Organic extracts were evaporated at high vacuum. To evaporate was added water with crushed ice and with vigorous stirring there was slowly added diluted solution of hydrogen peroxide (150-170 mol. %). Temperature is necessary to be kept between 3-5° C. Excess of hydrogen peroxide was decomposed by adding of spot manganese(IV) dioxide and by warming of mixture to 25° C. Residue has been chromatographed on alumina column after evaporation to ⅕ of volume. Thus 1.47 g of [2-(N-phthalimidyl)ethyl]-(1,4,7,10-tetraazacyclododec-1-ylmethyl)phosphinic acid ethyl ester has been obtained. Total yield: 67 percent.

### EXAMPLE 29

### Preparation of 1-[ethoxy-(4-nitrophenyl)phosphinoylmethyl]-1,4,8,11-tetraazacyclotetradecane and 11-[ethoxy-(4-nitrophenyl)phosphinoylmethyl]-1,4,8-triformyl-1,4,8,11-tetraazacyclotetradecane via 11-methoxymethyl-triformylcyclam

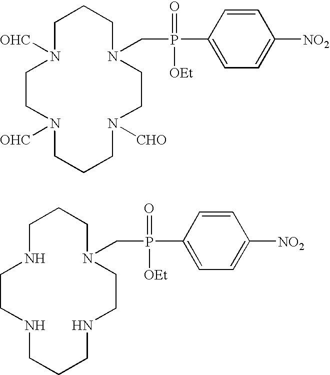

To a solution of 11-methoxymethyl-triformylcyclam (1.64 g, 5 mmol) (see Example 13d) was added 4-nitrophenylphosphinic acid ethyl ester (1.08 g, 5 mmol) in dry dimethylformamide (25 ml). This mixture was heated at 75° C. over the period of 2 hours. Thereafter reaction mass was evaporated at high vacuum near 75° C. To the residue was added water with crushed ice and with vigorous stirring has been slowly added diluted solution of hydrogen peroxide (150-170 mol. %). Temperature is necessary to be kept between 3-5° C. Excess of hydrogen peroxide was decomposed by adding of spot manganese(IV) dioxide and by warming of mixture to 25° C. Residue was chromatographed on alumina column after evaporation to ⅕ of volume. Thus, 1.56 g of 1-[ethoxy-(4-nitrophenyl)phosphinoylmethyl]-1,4,8,11-tetraazacyclotetradecane has been obtained. Total yield: 73 percent.

### EXAMPLE 30

### Preparation of tri-tert.-butyl ester [(3-benzyloxycarbonylamino-propyl)-ethoxy-phosphinoylmethyl]-1,4,7,10-tetraazacyclododecane-1,4,7,10-triacetic acid via (3-benzyloxycarbonylamino-propyl)-(1,4,7,10-tetraazacyclododec-1-ylmethyl)-phosphinic acid ethyl ester

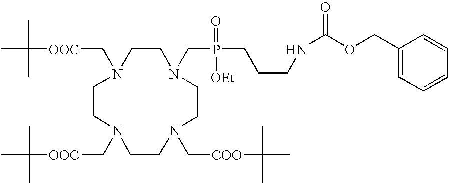

Into a 0.5 l three necked reaction vessel equipped with an addition funnel (with servo and pressure correction), electronic temperature meter bonded to thermostat, nitrogen overpressure inlet adapter and stirring apparatus, there were placed 5.4 g (11.5 mmol) of (3-benzyloxycarbonylamino-propyl)-(1,4,7,10-tetraazacyclododec-1-ylmethyl)-phosphinic acid ethyl ester and 120 ml of dried dimethylformamide. 3 grams (30 mmol) of pure potassium hydrogencarbonate and 2.5 grams (18 mmol) of ignitioned potassium carbonate were added thereafter. 9.2 grams (38 mmol) of tert.-butyl iodoacetate in 100 ml of dried dimethylformamide were added to the solution at 40° C. over the period of 4 hours. After addition, the mixture was stirred at 65° C. under nitrogen overpressure for 12 hours. Reaction mass was poured to 1.5 liters of 45° C. water with vigorous stirring. Separated oily product was chromatographed on AzaDVBP anex (triethylamine-tert.butylmethylether, 1:8 mixture). After evaporation of appropriate fractions solid product was obtained by recrystallization from mixture tert.-butanol-dichloromethane (1:2,5). Total yield: 87 percent.

### EXAMPLE 31

### Preparation of tri-tert.-butyl ester 11-[(3-benzyloxycarbonylamino-propyl)-ethoxy-phosphinoylmethyl]-1,4,8,11-tetraazacyclododecane-1,4,8-triacetic acid via (3-benzyloxycarbonylamino-propyl)-(1,4,8,11-tetraazacyclotetradec-1-ylmethyl)-phosphinic acid ethyl ester

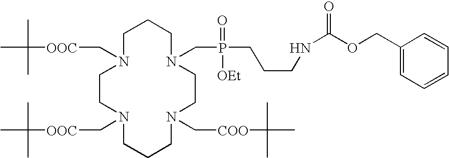

Tri-tert.-butyl ester 11-[(3-benzyloxycarbonylamino-propyl)-ethoxy-phosphinoylmethyl]-1,4,8,11-tetraazacyclododecane-1,4,8-triacetic acid was prepared from (3-benzyloxycarbonylamino-propyl)-(1,4,8,11-tetraazacyclotetradec-1-ylmethyl)-phosphinic acid ethyl ester by same method as it has been described in Example 30. Yield was 91 percent.

### EXAMPLE 32

### Preparation of tri-tert.-butyl ester 10-[ethoxy-(4-nitrobenzyl)phosphinoylmethyl]-1,4,7,10-tetraazacyclododecane-1,4,7-triacetic acid via ethyl ester

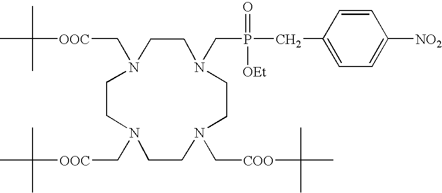

Into a 1 l three necked reaction vessel equipped with an addition funnel, electronic temperature meter bonded to thermostat, nitrogen overpressure inlet adapter and very power stirring apparatus, there were paced 14.5 g (19.18 mmol) of (4-nitrobenzyl)-(1,4,7,10-tetraazacyclododec-1-ylmethyl) phosphinic acid ethyl ester and 400 ml of dried acetonitrile and 0.5 g of TEBAC. 10.6 grams (76.7 mmol) of pure potassium carbonate in saturated aqueous solution were added thereafter. 15.3 grams (63.3 mmol) of tert.-butyl iodoacetate in 200 ml of acetonitrile were added to the emulsion at 40° C. with continuous vigorous stirring over period of 10 hours. After the addition, the mixture was vigorously stirred at 55° C. under nitrogen overpressure for 24 hours. Top layer was pumped off and concentrated at vacuo. Thereafter the residue was poured into 3 liters of 30° C. water with vigorous stirring. Oily product was triturated by hexane-toluene (2:1) mixture and chomatographed on AzaDVBP anex column (triethylamine-tert.butylmethylether, 1:10 mixture). Total yield: 76 percent.

### EXAMPLE 33

### Preparation of 11-[4-nitrobenzylphosphinoylmethyl]-1,4,8,11-tetraazacyclotetradecane-1,4,7-triacetic acid via 1-[ethoxy-(4-nitrobenzyl)phosphinoylmethyl]-1,4,8,11-tetraazacyclotetradecane

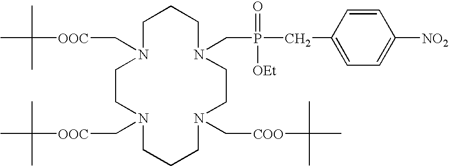

Into a 250 ml three necked reaction vessel equipped with an addition funnel, electronic temperature meter bonded to thermostat, nitrogen overpressure inlet adapter and high power stirring apparatus, there were placed 5.7 g (12.9 mmol) of 1-[ethoxy-(4-nitrobenzyl)phosphinoylmethyl]-1,4,8,11-tetraazacyclotetradecane and 30 ml of cold (<8-10° C.) aqueous ethanol (32.5%). 5 g grams (50 mmol) of pure potassium hydrogencarbonate in saturated aqueous solution were thereafter. Temperature was decreased to 0° C. and 95 grams (42.6 mmol) of potassium iodoacetate in 50 ml of cold water were added to the mixture with continuous vigorous stirring over the period of 14 hours. After the addition, the mixture was vigorously stirred at 25° C. under nitrogen low overpressure for another 24 hours. Probe to iodide must not indicate other iodide anion concentration increase. Mixture was quickly filtered and filtrate was concentrated at vacuo (4.5 kPa) to about 35-40 ml of mixture. Residue was acidified by conc. aqueous HCl to pH about 1-2 and mixture was refluxed for 12 hours. After evaporation at vacuo, residue was chromatographed on 3 l Dowex 50 (H⁺-form) column. Non-amine impurities were eluted with water, aminic compounds with aqueous ammonia. Eluents containing product were combined and evaporated at vacuo. After chromatography purification on Amberlite CG-50 (H⁺-form) column, 6.75 g of 11-[4-nitrobenzylphosphinoylmethyl]-1,4,8,11-tetraazacyclotetradecane-1,4,7-triacetic acid have been obtained in high purity (97% by HPLC) as oily product. Total yield: 89 percent.

### EXAMPLE 34

### Preparation of 10-[(4-nitrophenyl)-phosphono-methyl]-1,4,7,10-tetraazacyclododecane-1,4,7-triacetic acid via [(4-nitrophenyl)-(1,4,7,10-tetraazacyclododec-1-yl)-methyl]-phosphonic acid

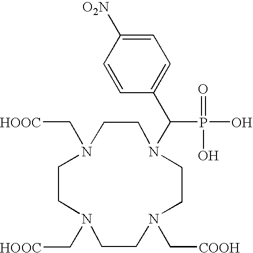

10-[(4-nitro-phenyl)-phosphono-methyl]-1,4,7,10-tetraazacyclododecane-1,4,7-triacetic acid was prepared from [(4-nitrophenyl)-(1,4,7,10-tetraazacyclododec-1-yl)-methyl]-phosphonic acid (see Example 19) by the same method as it has been described in Example 33. Yield was 76 percent.

### EXAMPLE 35

### Preparation of 10-(hydroxy-methyl-phosphinoylmethyl)]-1,4,7,10-tetraazacyclododecane-1,4,7-triacetic acid via methyl-(1,4,7,10-tetraazacyclododec-1-ylmethyl)-phosphinic acid ethyl ester

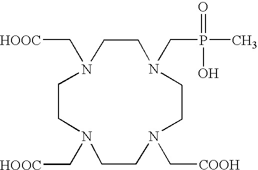

10-(hydroxy-methyl-phosphinoylmethyl)]-1,4,7,10-tetraazacyclododecane-1,4,7-triacetic acid was prepared from methyl-(1,4,7,10-tetraazacyclododec-1-ylmethyl)-phosphinic acid ethyl ester by the same method as it has been described in Example 33. Yield was 87 percent.

### EXAMPLE 36

### Preparation of [ethoxy-(1,4,7,10-tetraazacyclododec-1-ylmethyl)-phosphinoyl]-acetic acid ethyl ester via cyclene

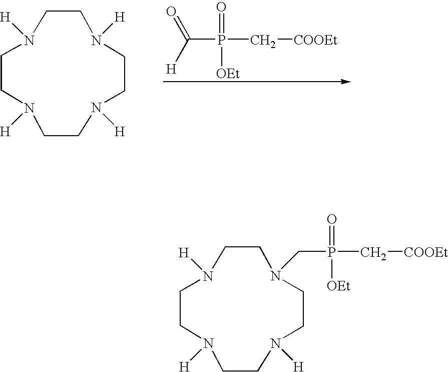

Into a 500 ml three necked reaction bottle equipped with an adding funnel, temperature meter, reflux condenser with argon overpressure inlet adapter and magnetic stirrer apparatus, there were placed 25.8 g (150 mmol) of cyclene and 31.22 g (150 mmol) of appropriate aldehyde in 160 ml of dried ethanol. To this solution there were added portion-by-portion very slowly 4.7 g (75 mmol) sodium cyanoborohydride at room temperature for 2 hours. After addition, the mixture was stirred under argon for the next 14 hours. 46.5 mg of high pure [ethoxy-(1,4,7,10-tetraazacyclododec-1-ylmethyl)-phosphinoyl]-acetic acid ethyl ester was obtained after ultra high vacuum evaporation, chromatography on AzaDVBP column (1 liter), trituration by chlorobenzene and recrystallization from acetonitrile-dichloroethane (1:1) mixture. Total yield: 85 percent.

### EXAMPLE 37

### Preparation of tribenzyl ester 10-[ethoxy-(2-methoxy-5-nitrobenzyl)phosphinoylmethyl]-1,4,7,10-tetraazacyclododecane-1,4,7-triacetic acid via (2-methoxy-5-nitrobenzyl)-(1,4,7,10-tetraazacyclododec-1-ylmethyl)phosphinic acid ethyl ester

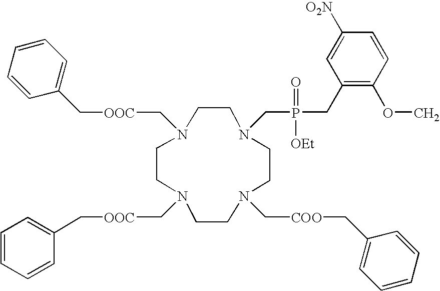

Into a 250 ml three necked reaction vessel equipped with an addition funnel (with servo and pressure correction), electronic temperature meter bonded to thermostat, nitrogen overpressure inlet adapter and strong stirring apparatus, there were placed 13 g (29.3 mmol) of (2-methoxy-5-nitrobenzyl)-(1,4,7,10-tetraazacyclododec-1-ylmethyl)phosphinic acid ethyl ester and 15 g (91.4 mmol) of benzyl glyoxylate in 100 ml of dried ethanol. The mixture was cooled down to 8° C. 5.5 grams (88 mmol) of sodium cyanoborohydride were added portion-by-portion over period of 2 hours thereafter. After adding was complete, temperature was raised to 20° C. Thereafter mixture was filtered and acidified with ice aqueous acetic acid to pH 6. After 24 hours standing at −5° C. it has been filtered once more. Now, reaction mass is concentrated at vacuo (12 kPa) to 30 ml approximately. Product is precipitated by adding of 800 ml of water. Solid matter was filtered, dried and recrystallized from ethanol-hexane (2:1) mixture. Thus recrystallization affords 22.4 g of tritbenzyl ester 10-[ethoxy-(2-methoxy-5-nitrobenzyl) phosphinoylmethyl]-1,4,7,10-tetraazacyclododecane-1,4,7-triacetic acid as pale yellow product in 86% yield.

### EXAMPLE 38

### Preparation of triethyl ester (4-nitrophenyl-ethoxy-phosphinoylmethyl)-1,4,7,10-tetraazacyclododecane-1,4,7-triacetic acid via {bis-[2-(toluene-4-sulfonyloxy)ethyl]-amino}-acetic acid ethyl ester (A) and (2-{[2-(ethoxycarbonylmethyl-amino)-ethyl]-[ethoxy-(4-nitrophenyl)-phosphinoylmethyl]-amino}-ethylamino)-acetic acid ethyl ester (B)

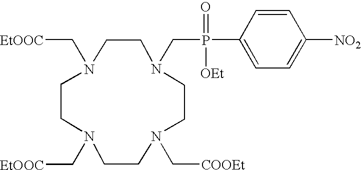

Into a 8000 ml three necked reaction bottle equipped with two adding input from dual peristaltic pump, temperature meter, reflux condenser with argon overpressure inlet adapter and a strong mechanic stirrer apparatus, there were placed 1800 ml of toluene and mixture from 6.8 g (8 mol. %) of tetrabutylammonium hydrogensulphate, 46.2 g (0.55 mol) of sodium hydrogencarbonate and 500 ml of water. With continuous vigorous stirring and heating to reflux temperature, both reactants (0.25 mol; 124.9 g of A, 125.6 g of B) as solutions in one liter of dioxane were added by peristaltic pump over the period of 48 hours. After addition, the mixture has been stirred under argon and same temperature for the next 6 hours. Toluene layer was separated, washed by 10% aqueous calcium chloride solution and by water, dried over mol. sieve and evaporated at vacuo. Dark brown product was chromatographed on glycolmethacrylate catex column. Thus have been obtained 29.6 g of product triethyl ester (4-nitrophenyl-ethoxy-phosphinoylmethyl)-1,4,7,10-tetraazacyclododecane-1,4,7-triacetic acid (18%) after elution by anhydrous ethanol-triethylamine mixture.

### EXAMPLE 39

### Preparation of DO3A-methylarsenic acid via 1,4,7,10-tetraazacyclododecane-1,4,7-triacetic acid (DO3A)

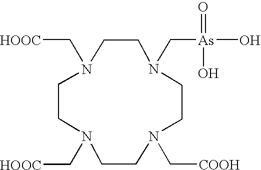

Chloromethylarsenic acid (5 g, 28.7 mmol) was added to a solution of DO3A 8.66 g (25 mmol) in distilled water (25 ml) and pH of the solution was adjusted to 10 (solid LiOH). The mixture was heated to 45° C. for 48 h with periodic addition of solid LiOH to maintain the pH>9.5. After having cooled and been acidified to pH 2 conc. hydrochloride acid the solution was evaporated to approximately 8 ml, and ethanol (40 ml) was added to give white gum. After decantation of the settled supernatant liquid, the residue was redissolved in water (3 ml) and ethanol was added (15 ml) slowly and two layers were allowed to diffuse together. 5 g crystalline solid of DO3A-methylarsenic acid were obtained after 24 h standing. Total yield: 42 percent.

### EXAMPLE 40

### Preparation of 3a-[ethoxy-(4-nitrobenzyl)-phosphinoylmethyl]decahydro-5a,8a,10a-triaza-3a-azonia-pyrene trifluoromethanesulfonate via decahydro-, 3a,5a,8a,10a-tetraazaprene

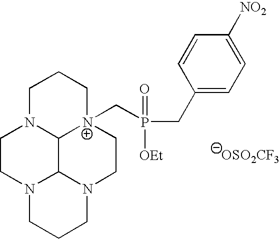

In 100 flask equipped by magnetic stirrer there were dissolved 3 g (13.5 mmol) of decahydro-3a,5a,8a,10a-tetraazapyrene in 25 ml of absolutised toluene (freshly distilled from activated calcium). With continues vigorous stirring 5.28 g (13.5 mmol) of trifluoromethanesulfonic acid ethoxy-(4-nitrobenzyl)-phosphinoylmethyl ester in 20 ml of absolutised toluene were added to this solution. Mixture was stirred for the next 4 hours after addition of all reactants. Temperature was maintained at 50° C. Thereafter, reaction mixture was cooled to 5° C. and product was filtered. Solid mass was triturated with 150 ml of diethylether and filtered against. Product was dried over phosphorus pentoxide about 2 days. Thus, 5 g of 3a-[ethoxy-(4-nitrobenzyl)-phosphinoyl methyl]decahydro-5a,8a,10a-triaza-3a-azonia-pyrene trifluoromethanesulfonate were obtained. Total yield: 61 percent.

### EXAMPLE 41

### Preparation of (4-nitrobenzyl)-(1,4,8,11-tetraazacyclotetradec-1-ylmethyl)-phosphinic acid via 3a-[ethoxy-(4-nitrobenzyl)-phosphinoylmethyl]decahydro-5a,8a,10a-triaza-3a-azonia-pyrene trifluoromethanesulfonate

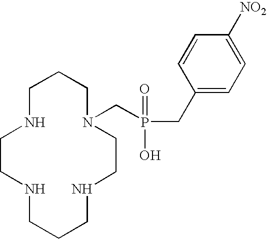

In 100 ml bottle equipped with magnetic stirrer and reflux condenser with argon inlet apparatus 3 g (4.9 mmol) 3a-[ethoxy-(4-nitrobenzyl)-phosphinoylmethyl]decahydro-5a,8a, 10a-triaza-3a-azonia-pyrene trifluoromethanesulfonate were heated with 13 ml of 100% hydrazine hydrate over the period of 2.5 hours. Thereafter, 10 ml of water were added to this mixture. Excess of hydrazine was evaporated at high vacuo (0.1 torr). To the residue there were added 15 ml of concentrated hydrochloric acid and this solution was refluxed 12 hours. After refluxing was stopped, mixture was evaporated to dryness. After evaporation at vacuo, residue was chromatographed on 1 l Dowex 50 (H⁺-form) column. Non-amine impurities were eluted with water, aminic compounds with aqueous ammonia. Eluents containing product were combined and evaporated at vacuo. After chromatography purification on Amberlite CG-50 (H⁺-form) column, 1.36 g of (4-nitrobenzyl)-(1,4,8,11-tetraazacyclotetradec-1-ylmethyl) phosphinic acid was obtained in good purity (96% by HPLC) as yellowish crystalline product. Total yield: 67 percent.

### EXAMPLE 42

### Preparation of 4,7,10-Tris-carboxymethyl-4,7,10-triaza-1-azonia-cyclododecane hypophosphite and 10-hydroxyphosphinoylmethyl-1,4,7,10-tetraazacyclododecane-1,4,7-triacetic acid via 1,4,7,10-tetraazacyclododecane-1,4,7-triacetic acid (DO3A)

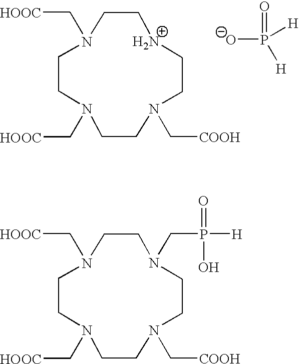

a) To 1.32 g of 50% (0.66 g anhydrous H₃PO₂, 10 mmol) aquoeous solution of hypophosphorous acid in 5 ml of water there were added 3.46 g (10 mmol) of DO3A in 100 ml flask equipped by septum and adding funnel. After 10 minutes stirring, this solution was added to 200 ml of anhydrous ethanol. The precipitated salt were filtered off and washed with dry ethanol. Thus semiproduct was obtained in crystalline form. After filtration and drying, 3.9 grams of 4,7,10-tris-carboxymethyl-4,7,10-triaza-1-azonia-cyclododecane fosfornan were obtained. Total yield: 95 percent.

b) 2.06 g (5 mmol) of 4,7,10-tris-carboxymethyl-4,7,10-triaza-1-azonia-cyclododecane hypophosphite was mixed with 180 mg (6 mmol) of paraformaldehyde in PTFE autoclave. The mixture was irradiated with microwaves of 700 W power in reflex microwave reactor for 1 minute. 20 ml of water were added to PTFE bottle after opening of apparatus and mixture was evaporated to dryness. After evaporation at vacuo, residue was chromatographed on 500 ml Dowex 50 (H⁺-form) column. Non-amine impurities were eluted with water, aminic compounds with aqueous ammonia. Eluents containing product were combined and evaporated at vacuo. After chromatography purification on Amberlite CG-50 (H⁺-form) column 1.74 g of 10-hydroxyphosphinoylmethyl-1,4,7,10-tetraazacyclododecane-1,4,7-triacetic acid was obtained as pale yellow crystalline product. Total-yield: 82 percent.

### EXAMPLE 43

### Preparation of 2-[7-(diethoxyphosphorylmethyl-11,12-dioxo-1,4,7,10-tetraazabicyclo[8.2.2]tetradec-4-yl]-3-(4-nitrophenyl)propionic acid ethyl ester and 2-(4,7-bis-carboxymethyl-10-phosphonomethyl-1,4,7,10-tetraazacyclododec-1-yl)-3-(4-nitrophenyl)-proprionic acid via (11,12-dioxo-1,4,7,10-tetraaza bicyclo[8.2.2]tetradec-4-ylmethyl)phosphonic acid diethyl ester

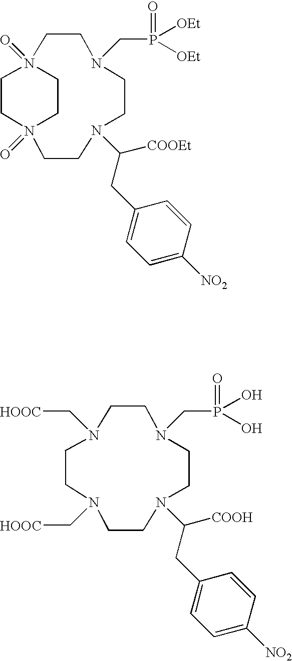

2-[7-(diethoxyphosphorylmethyl)-11,12-dioxo-1,4,7,10-tetraazabicyclo[8.2.2]tetradec-4-yl]-3-(4-nitrophenyl)propionic acid ethyl ester was prepared by reaction of 2-Bromo-3-(4-nitrophenyl)propionic acid ethyl ester with (11,12-dioxo-1,4,7,10-tetraazabicyclo[8.2.2]tetradec-4-ylmethyl)phosphonic acid diethyl ester in presence of base (extraction to organic phase). Acid hydrolysis of 2-[7-(diethoxyphosphorylmethyl)-11,12-dioxo-1,4,7,10-tetraazabicyclo[8.2.2]tetradec-4-yl]-3-(4-nitrophenyl)propionic acid ethyl ester by aqueous hydrochloric acid affords 2-(4,7-bis-carboxymethyl-10-phosphonomethyl-1,4,7,10-tetraazacyclododec-1-yl)-3-(4-nitrophenyl)-propionic acid. The separation of product is similar as it has been described in Example 42.

### EXAMPLE 44

### Preparation of 10-(carboymethyl-hydroxy-phosphinoylmethyl)-1,4,7,10-tetraazacyclododecane-1,4,7-triacetic acid via 10-hydroxyphosphinoylmethyl-1,4,7,10-tetraazacyclododecane-1,4,7-triacetic acid

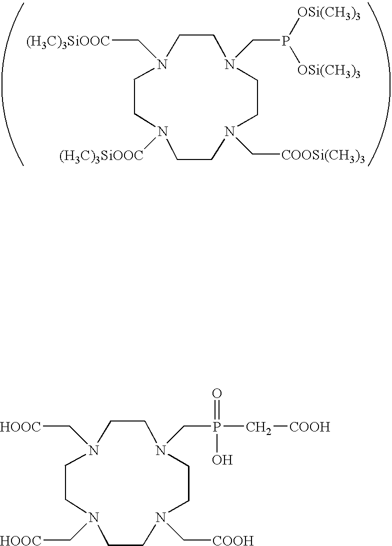

In 250 ml flask equipped with reflux condenser there were refluxed 5 g (11.78 mmol, Example 42) of 10-hydroxyphosphinoylmethyl-1,4,7,10-tetraazacyclododecane-1,4,7-triacetic acid with 1200 mol. % of hexamethyldisilazane under argon for 12 hours. Thereafter, 3 g (14.14 mmol) of trimethylsilyl bromoacetate in absolute dichloromethane were added slowly over the period of 20 minutes and then stirred for another 6 hours. The mixture was evaporated and to the residue was added 50 ml of water. Solution was chromatographed on 600 ml Dowex 50 (H⁺-form) column. Non-amine impurities were eluted with water, aminic compounds with aqueous ammonia. Eluents containing product were combined and evaporated at vacuo. After chromatography purification on Amberlite CG-50 (H⁺-form) column, 3.3 g of 10-(carboxymethyl-hydroxy-phosphinoylmethyl)-1,4,7,10-tetraazacyclododecane-1,4,7-triacetic acid was obtained as colorless crystalline product. Total yield: 58 percent.

### EXAMPLE 45

### Preparation of heptaethyl ester 10-[bis(phosphono)methyl]-1,4,7,10-tetraazacyclododecane-1,4,7-triacetic (Et₇DO3AP2) and 10-[bis(phosphono)methyl]-1,4,7,10-tetraazacyclododecane-1,4,7-triacetic (DO3AP2) via triethyl ester 1,4,7,10-tetraazacyclododecane-1,4,7-triacetic acid (Et₃DO3A)

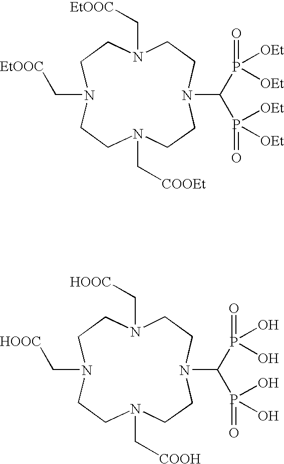

a) A mixture of Et₃DO3A 22.8 g (53 mmol), triethyl orthoformate 9.3 g (63 mmol) and diethyl phosphite 29.3 g (212 mmol) was stirred at 140° C. for 1.5 h with continuous removal of the ethanol formed. After cooling the volatiles were removed in vacuo. The residue was chromatographed on silica gel using CHCl₃—MeOH—NH₃ (49:1:1) as an eluent to give 30.4 g Et₇DO3AP2 as a pale yellow oil. Total yield: 80 percent.

b) A solution of Et₇DO3AP2 (21.5 g, 30 mmol, Example 45a) in concentrated hydrochloric acid (200 ml) was stirred under reflux for 3 h, and then the solvent was evaporated off in vacuo. 50 ml of water was added to the residue. Solution was chromatographed on 1000 ml Dowex 50 (H⁺-form) column. Non-amine impurities were eluted with water, aminic compounds with aqueous ammonia. Eluents containing product were combined and evaporated at vacuo. After chromatography purification on Amberlite CG-50 (H⁺-form) column, 14.67 g of DO3AP2 were obtained as colorless crystalline product. Total yield: 94 percent. TLC indicated one spot.

### EXAMPLE 46

### Preparation of tri-tert.-butyl ester 10-{methoxy-[2-methoxycarbonyl-3-(4-nitrophenyl)propyl]phosphinoylmethyl}-1,4,7,10-tetraazacyclododecane-1,4,7-triacetic acid via tri-tert.-butyl ester 10-(ethoxyphosphinoylmethyl)-1,4,7,10-tetraazacyclododecane-1,4,7-triacetic acid

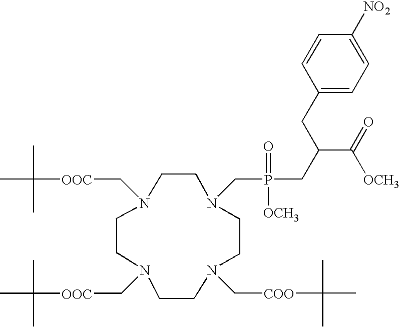

To the solution containing 3.03 g (5 mmol) tri-tert.-butyl ester 10-(ethoxyphosphinoylmethyl)-1,4,7,10-tetraazacyclododecane-1,4,7-triacetic acid and 25 ml dry THF, was added, dropwise, at 0° C., first a solution of 1.2 g (5.5 mmol) of 2-(4-nitrobenzyl)acrylic acid methyl ester in 25 ml dry THF, then, 56 mg (0.5 mmol) of a solution of t-BuOK in 5 ml dry THF. The reaction mixture was then stirred for 3 h at 0° C. and at r.t. overnight. The reaction was quenched with 0.5 N HCl and extracted with EtOAc. The organic phase was dried over molecular sieve 4A and evaporated under vacuum. After chromatography purification on silica gel with CHCl₃-hexane, the product was obtained as a viscous oil. Total yield: 78 percent. TLC indicated one spot.

1. Method of preparation or synthesis of ligands of the general formula 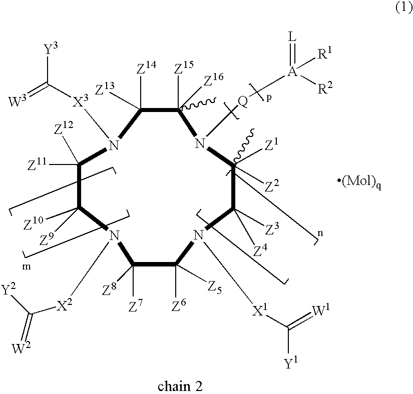
wherein:
A is phosphorus or arsenic;
Z¹⁻¹⁶ is independently selected from a radical of hydrogen; chlorine; bromine; fluorine; iodine; nitro or nitroso; sulpho; or a substituted or unsubstituted straight-chained, branched or cyclic hydrocarbon radical having from 1 to 20 carbon atoms and being saturated or unsaturated with one or more double or triple bonds and optionally containing heteroatoms such as F. Br, C₁₋₁₀, N, S and/or P; a substituted or unsubstituted aromatic radical having from 5 up to 18 ring carbon atoms or its aryloxy derivative and including polynuclear aromatic radicals; hydroxyl; alkoxyl; S-substituted or S-unsubstituted thiol; mono- or disubstituted or unsubstituted amine; Z¹⁻¹⁶ also can constitute independently carbonyl and general functional derivatives of carbonyl as oxime, hydrazone, but especially N-substituted or unsubstituted carboimidyl; thiocarbonyl; condensed substituted or unsubstituted benzoderivative; A(L) R¹R²;
n, m is independently 1 or 2;
X¹⁻³ is independently methylene or ethylene substituted as defined for Z¹⁻¹⁶ especially with or without heteroatoms and multiple bonds; carbonyl; N-substituted or unsubstituted carboimidyl; thiocarbonyl;
Y¹⁻³ is independently methyl substituted as defined for Z¹⁻¹⁶; hydroxyl; O-substituted hydroxyl with Z¹⁻¹⁶; S-substituted thiol; substituted or unsubstituted amine; hydroxylate or thiolate of metal cations or organic cations such as Na, Li, K, Rb, Cs, Ca, Mg, Al, Zn, Mn, Cr, Mo, ⁶⁴Cu, ⁶⁷Cu, ⁶⁷Ga, ⁹⁰Y, ¹¹¹In, ¹⁵³Sm, ¹⁶⁶Ho, ¹⁷⁷Lu, ²⁰¹Tl, ²¹²Bi, ammonium, primary, secondary, tertiary and quarternary alkyl and aryl ammonium, sulphonium and phosphonium salts and their combinations; Y¹⁻³ can constitute independently a substituted hydroxylamine of formula: 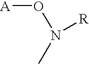
wherein A is independently methyl substituted as defined for Z¹⁻¹⁶; a metal cation or organic cation such as Na, Li, K, Rb, Cs, Ca, Mg, Al, Zn, Mn, Cr, Mo, ⁶⁴Cu, ⁶⁷Cu, ⁶⁷Ga, ⁹⁰Y, ¹¹¹In, ¹⁵³Sm, ¹⁶⁶Ho, ¹⁷⁷Lu, ²⁰¹Tl, ²¹²B, ammonium, primary, secondary, tertiary and quarternary alkyl and aryl ammonium, sulphonium and phosphonium salts and their combinations;
R is independently a radical of hydrogen; substituted or unsubstituted straight-chained, branched or cyclic hydrocarbon radical having from 1 to 20 carbon atoms and being saturated or unsaturated with one or more double or triple bonds and optionally containing heteroatoms such as F, Br, Cl, N, S and/or P; a substituted or unsubstituted aromatic radical having from 5 up to 18 ring carbon atoms and including polynuclear aromatic radicals;
Q is independently methylene or ethylene substituted as defined in Z¹⁻¹⁶, ethenylene or ethynylene substituted as defined in Z¹⁻¹⁶; carbonyl; N-substituted or unsubstituted carboimidyl; thiocarbonyl;
p is from 1 to 10;
R¹⁻² is independently hydrogen; halogen; substituted or unsubstituted straight-chained, branched or cyclic hydrocarbon radical having from 1 to 20 carbon atoms and being saturated or unsaturated with one or more double or triple bonds and optionally containing heteroatoms such as F, Br, C₁₋₁₀, N, S and/or P; substituted or unsubstituted aromatic radical having from 5 up to 18 ring carbon atoms or its aryloxy derivative and including polynuclear aromatic radicals; hydroxyl; alkoxyl; thiol; thioalcoxyl; substituted or unsubstituted amine; trialkylsilyl; trialkylsilyloxy, triarylsilyl; triarylsilyloxy; hydroxylate or thiolate of metal cations or organic cations such as Na, Li, K, Rb, Cs, Ca, Mg, Al, Zn, Mn, Cr, Mo, ⁶⁴Cu, ⁶⁷Cu, ⁶⁷Ga, ⁹⁰Y, ¹¹¹In, ¹⁵³Sm, ¹⁶⁶Ho, ¹⁷⁷Lu, ²⁰¹Tl, ²¹²Bi, ammonium, primary, secondary, tertiary and quarternary alkyl and aryl ammonium, sulphonium and phosphonium salts and their combinations;
L is oxygen, sulphur, N-substituted or unsubstituted imidyl;
W¹⁻³ is independently oxygen, sulphur, N-substituted or unsubstituted imidyl;
Mol is a protogenic acid, for example, a mineral acid, a substituted or unsubstituted carboxylic, sulphonic, phosphonic and phosphinic acid or a protophilic base, for example, pyridine, tetrahydrofurane, triethylphosphine or a Lewis acid, for example, BF₃, ZnCl₂, AlCl₃, FeBr₃ or a neutral molecule bonded as e.g. in molecular cluster or associate, e.g. chloroform, toluene, water, dioxan, aceton, dimethylformamide cyclodextrine, calix[8]arene, polyethyleneglycole 800;
q is a number from 0 to 10 including a fraction number such as ½ or ⅔ or ¾, 4/3, 3/2.
from unitriprotected intermediates (N4 GH or N4G- or N4H4(Me)w(X)u) of structure: 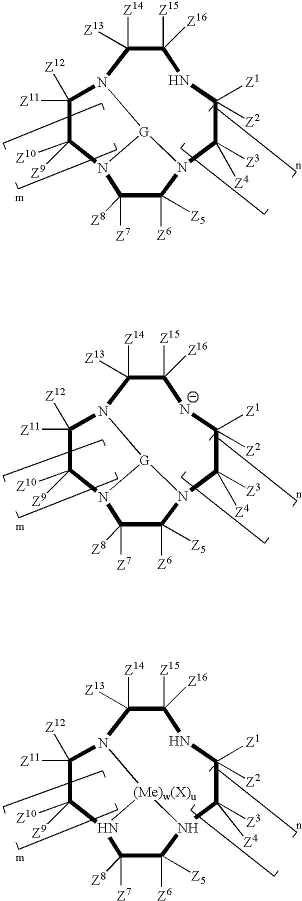
wherein G is CZ¹⁻¹⁶ (include C⁺ as carbocation with Z¹⁻¹⁶ as anion), SiZ¹⁻¹⁶, SnZ¹⁻¹⁶, B, Al, P, As, PO, AsO, PS, AsS, AsZ¹⁻¹⁶, VZ¹⁻¹⁶, PZ¹⁻¹⁶;
Me is metal (or ion), especially: Cu, Ni, Fe, Zn, Cr, Mo, V; X is ligand for example Cl, Br, OH, etc.; u is from 1 to 15; w is 1 or 2 or 3 or ½ or ⅔ or 3/2), or formates TiOZ¹⁻¹⁶, TiNZ¹⁻¹⁶, MoP, MoN;
by condensation with Subst-(Q)ₚA(L)(R¹)(R²) of structure: 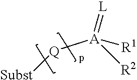
wherein Subst is general leaving group, of structure: —OR, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, wherein Rⁱ⁻ⁱⁱⁱ are groups of the same type as R. (Subst is for example: hydroxyle, alcoxyl, aryfoxyl, alkyl amine, N,N-dialkylamine, N-alkylamine, arylsulfonyloxy, tosyloxy, mesyloxy, triflyloxy, acetoxy, benzoyloxy, N-benztriazolyl, trialkylsilyloxy, hydrazine and N-substituted hydrazine, benzyloxycarbonyloxy, tert.-butyloxycarbonyloxy 1-imidazolyl, succinimidyloxy, N-succinimidyl, N-phthalimidyl, N-phthalimidyloxy, arylthio, thiole, S-alkylthiole etc.), or group which generates in situ or on introduced functional group (Q)ₚA(L)(R¹)(R²) with or without isolation cation or partial positive charge with capability to reaction (photochemically, thermically or electrochemically cleavable groups)
under conditions of general nucleofilic substitution: especially under conditions of phase-transfer catalysis, in aprotic polar solvents or its mixtures (as dimethylformamide or dimethylacetamide or acetonitrile, dimethylsulphoxide or sulpholane or hexamethylphosphortriamide), in micellar medium, in solidphase (for example bonded N₄G⁻ on anex), with or without microwave irradiation, with or without ultrasonic irradiation, under conditions of high pressure (for example in autoclave), in aqueous phase in presence of pH-buffer, in milieu of water-free solvents with or without presence of base (for example: amines, aldimines, carbonates, fluorides, thioethers), enzymatic catalysis, in presence of dehydrating agent or agent reacting with protogenic product reaction or in presence of Lewis acid (e.g. ZnCl₂, BF₃, Et₂O, SiCl₄) etc.
and by next partially or full cleaving of G or (Me)_{w}(X)ᵤ. Both the steps can be solved as one-step reaction or separately.

2. Method of preparation or synthesis of ligands of the general formula (1) as defined in claim 1 from triprotected intermediates (N₄ GH or N₄G⁻ or N₄H₄(Me)_{w}(X)ᵤ) of the same structure as in claim 1
by addition on (Q)A(L)(R¹)(R²) of structure: 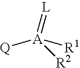
wherein anywhere on Q is double or multiple bond with capability to add intermediates (N₄GH or N₄G⁻) under conditions of general addition: especially carried out under high-pressure (for example in autoclave), microwave irradiation, under reflux in high boiling solvents, in micellar systems, in solid phase, under cryogenic conditions, under phase transfer catalysis, etc.
Double or multiple bonds can be generated in situ with or without isolation by general elimination methods from (Q-(XY)ₙ)A(L)(R¹)(R²) by elimination of XY, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc.
n is from 1 to 12.
Double or multiple bonds on (Q)A(L)(R¹)(R²) can be generated also from other substituents, which constitute (Q)A(L)(R¹)(R²) under conditions of irradiation (include thermic) or electrochemical reactions, e.g. tetrazenes, cyclic azides, triazenes, dixandiones etc.
or by addition and subsequent reduction (or in situ reduction) on an intermediate R—C(═W¹⁻³)(Q)ₚₚA(L)(R¹)(R²) of structure: 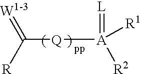
wherein pp is from 0 to 9.

3. Method of preparation or synthesis of ligands of the general formula (1) as defined in claim 1 from unitriprotected intermediates of structure: 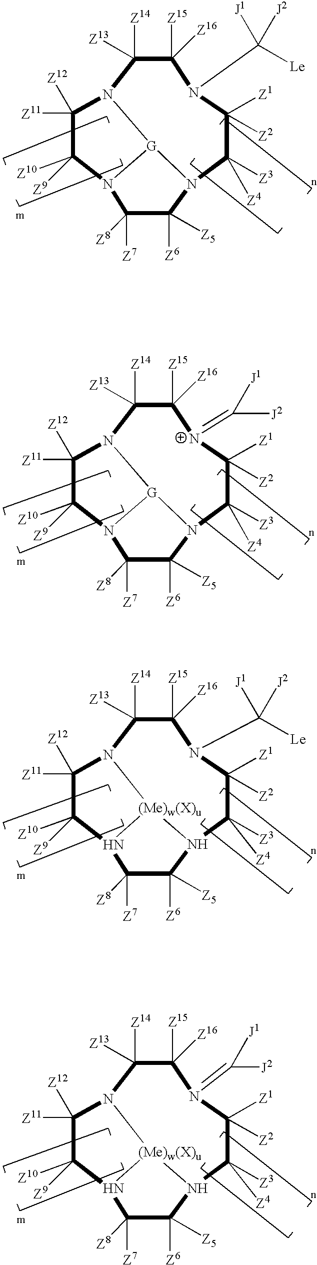
wherein:
G is CZ¹⁻¹⁶ (include C⁺ as carbocation with Z¹⁻¹⁶ as anion), SiZ¹⁻¹⁶, SnZ¹⁻¹⁶, B, Al, P, As, PO, AsO, PS, AsS, AsZ¹⁻¹⁶, VZ¹⁻¹⁶, PZ¹⁻¹⁶;
Me is metal (or ion), especially: Cu, Ni, Fe, Zn, Cr, Mo, V; X is ligand for example Cl, Br, OH, etc.; u is from 1 to 15;
w is 1 or 2 or 3 or ½ or ⅔ or 3/2), or formates TiOZ¹⁻¹⁶, TiNZ¹⁻¹⁶, MoP, MoN;
J¹⁻² is group (substituent, fragment) of the same type as Z¹⁻¹⁶ (J¹⁻² can form substituted methylen) and Le is leaving group, especially of structure: —OR, —OH, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, N-benztriazolyl, 1-imidazolyl, succinimidyloxy, N-succinimidyl, N-phthalimidyl, N-phthalimidyloxy wherein Rⁱ⁻ⁱⁱⁱ are groups of the same type as R
by reaction (e.g. condensation) with precursors or their mixture of structure: 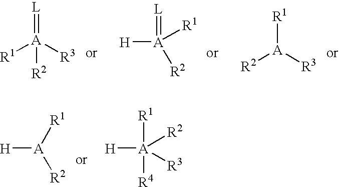
wherein R³ and R⁴ are groups of the same type as R¹⁻². Especially can be used: alkylphosphinic acid, arylphosphinic acid, trialkylphosphites, triarylphosphites, trialkylphosphines, triarylphosphines, dialkylphosphinates, diarylphosphinates, alkylarylphosphinates, dialkylarylphosphites, alkyldiarylphosphites, phosphinic acid, alkylarsenic(III) acid, arylarsenic(III) acid, trialkylarsenic(III), triarylarsenic(III), etc.
under conditions of general nucleofilic substitution: especially under conditions of phase-transfer catalysis, in aprotic polar solvents or its mixtures (as dimethylformamide or dimethylacetamide acetonitrile, dimethylsulphoxide or sulpholane or hexamethylphosphortriamide), in micellar medium, in solidphase (for example bonded N₄G⁻ on anex), with or without microwave irradiation, with or without ultrasonic irradiation, under conditions of high pressure (for example in autoclave), in aqueous phase in presence of pH-buffer, in milieu of water-free solvents with or without presence of base (for example: amines, aldimines, anex, carbonates, fluorides, thioethers), enzymatic catalysis, in presence of dehydrating agent or agent reacting with protogenic product reaction or in presence of Lewis acid as catalysators (for example ZnCl₂, BF₃.Et₂O, SiCl₄) etc.

4. Method of preparation or synthesis of ligands of the general formula (1) as defined in claim 1 from triprotected intermediates (N₄(Prot)₃H/N₄ (Prot)₃⁻ or N₄(XC(W)(Y))₃H/N₄(XC(W)(Y))₃⁻) of structure: 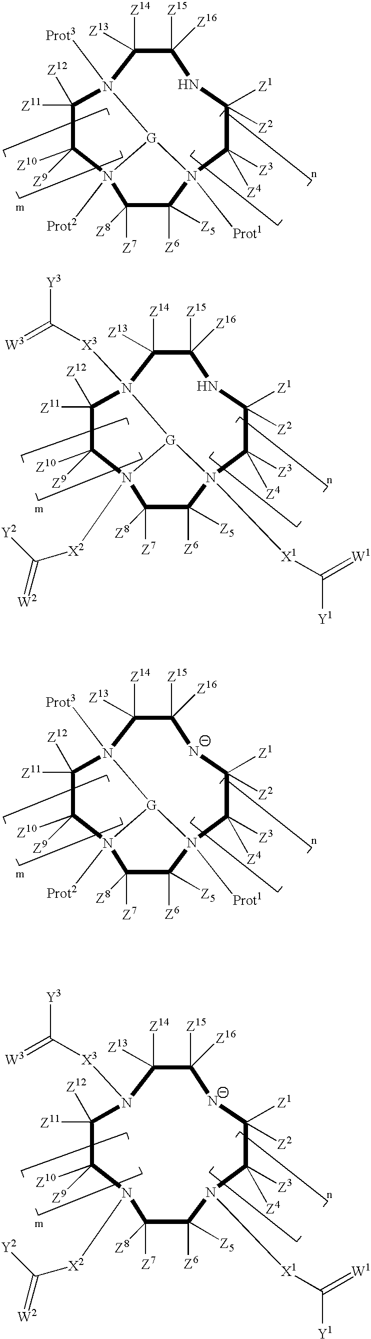
wherein Prot¹⁻³ is independently protective group (or electron pair with negative charge) especially of general structure: —CHO, —COR, —COOR, —CONRⁱRⁱⁱRⁱⁱⁱ, —SO₂R, —SR, —R, —SiRⁱRⁱⁱRⁱⁱⁱ, —PORⁱRⁱⁱ, —PSRⁱRⁱⁱ, —PO(ORⁱ)(ORⁱⁱ); protective groups Prot¹ and Prot² or Prot¹ and Prot³ may be also connected to each other especially according to following general structure: —CRⁱRⁱⁱ—, —CO—, —COCO—, —CS—, —C(═NR)—, —COCRⁱRⁱⁱCO—, —CO—R—CO—, [—CRⁱRⁱⁱCRⁱRⁱⁱ]₂SO, [—CRⁱRⁱⁱCRⁱRⁱⁱ]₂SO₂, [—CRⁱRⁱⁱCRⁱRⁱⁱ]₂P(O)OR, [—CRⁱRⁱⁱCRⁱRⁱⁱ]₂NR, —PO(OR)—, —SiRⁱRⁱⁱ—, —SnRⁱRⁱⁱ— wherein Rⁱ⁻ⁱⁱⁱ are groups of the same type as R. Prot¹⁻³ is for example methanesulphonyl, 4-toluenesulphonyl, trifluoromethanesulphonyl, nitrobenzenesulphonyl, benzenesulphonyl, naphthalenesulphonyl, formyl, acetyl, benzoyl, phthaloyl, trifluoroacetyl, tert.-butoxycarbonyl (BoC), 9H-fluoren-9-yl-methoxycarbonyl (Fmoc), benzyloxycarbonyl (Z), ethoxycarbonyl (Boc), methoxycarbonyl (Meoc), methoxybenzylcarbonyl (Moz), trityl, benzyl, benzhydryl, 4,4′-dimethoxytrityl, 4-methoxybenzoyl, ethandioyl, propandioyl, carbonyl, thiocarbonyl etc.
by condensation with Subst-(Q)ₚA(L)(R¹)(R²) of structure: 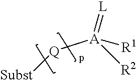
wherein Subst is general leaving group, of structure: —OR, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, wherein Rⁱ⁻ⁱⁱⁱ are groups of the same type as R. (Subst is for example: hydroxyle, alcoxyl, aryloxyl, alkyl amine, N,N-dialkylamine, N-alkylamine, arylsulfonyloxy, tosyloxy, mesyloxy, triflyloxy, acetoxy, benzoyloxy, N-benztriazolyl, trialkylsilyloxy, hydrazine and N-substituted hydrazine, benzyloxycarbonyloxy, tert.-butyloxycarbonyloxy, 1-imidazolyl, succinimidyloxy, N-succinimidyl, N-phthalimidyl, N-phthalimidyloxy, arylthio, thiole, S-alkylthiole etc.), or group which generates in situ or on introduced functional group (Q)ₚA(L)(R¹)(R²) with or without isolation cation or partial positive charge with capability to reaction (photochemically, thermically or electrochemically cleavable groups) under conditions of general nucleofilic substitution: especially under conditions of phase-transfer catalysis, in aprotic polar solvents or its mixtures (as dimethylformamide or dimethylacetamide acetonitrile, dimethylsulphoxide or sulpholane or hexamethylphosphortriamide), in micellar medium, in solidphase (for example bonded N₄G⁻ on anex), with or without microwave irradiation, with or without ultrasonic irradiation, under conditions of high pressure (for example in autoclave), in aqueous phase in presence of pH-buffer, in milieu of water-free solvents with or without presence of base (for example: amines, aldimines, anex, carbonates, fluorides, thioethers), enzymatic catalysis, in presence of dehydrating agent or agent reacting with protogenic product reaction or in presence of Lewis acid as catalysators (for example ZnCl₂, BF₃.Et₂O, SiCl₄) etc.
and by next possible partially or full cleaving of Prot¹⁻³. Both the steps can be solved as one-step reaction or separately.

5. Method of preparation or synthesis of ligands of the general formula (1) as defined in claim 1 from triprotected intermediates (N₄(Prot)₃H/N₄ (Prot)₃⁻ or N₄(XC(W)(Y))₃H/N₄(XC(W)(Y))₃⁻) of the same structure as in claim 4
by addition on (Q)A(L)(R¹)(R²) of structure: 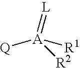
wherein anywhere on Q is double or multiple bond with capability to add intermediates (N₄(Prot)₃H./N₄(Prot)₃⁻ or N₄(XC(W)(Y))₃H./N₄(XC(W)(Y))₃⁻) under conditions of general addition: especially carried out under high-pressure (for example in autoclave), microwave irradiation, under reflux in high boiling solvents, in micellar systems, in solid phase, under cryogenic conditions, under phase transfer catalysis, etc.
Double or multiple bonds can be generated in situ with or without isolation by general elimination methods from (Q-(XY)ₙ)A(L)(R¹)(R²) by elimination of XY, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc. n is from 1 to 12.
Double or multiple bonds on (Q)A(L)(R¹)(R²) can be generated also from other substituents, which constitute (Q)A(L)(R¹)(R²) under conditions of irradiation (include thermic) or electrochemical reactions, e.g. tetrazenes, cyclic azides, triazenes, dixandiones etc.
or by addition and subsequent reduction (or in situ reduction) on an intermediate R—C(═W¹⁻³)(Q)ₚₚA(L)(R¹)(R²) of structure: 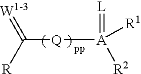
wherein pp is from m to 9.

6. Method of preparation or synthesis of ligands of the general formula (1) as defined in claim 1 from triprotected intermediates with active methylene or methylidene group of structure: 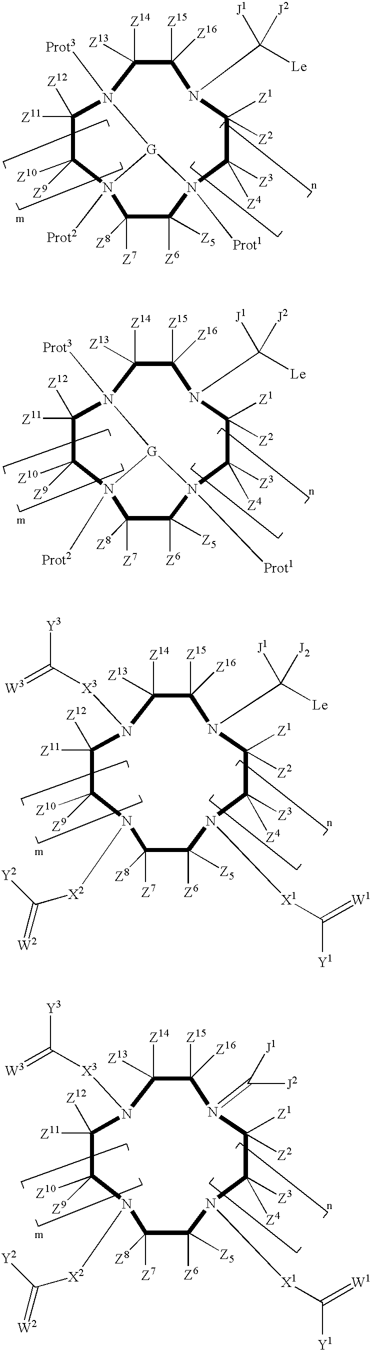
wherein J¹⁻² is group (substituent, fragment) of the same type as Z¹⁻¹⁶ and Le is leaving group, especially of structure: —OR, —OH, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, N-benztriazolyl, 1-imidazolyl, succinimidyloxy, N-succinimidyl, N-phthalimidyl, N-phthalimidyloxy wherein Rⁱ⁻ⁱⁱⁱ are groups of the same type as R
Prot¹⁻³ is independently protective group (or electron pair with negative charge) especially of general structure: —CHO, —COR, —COOR, —CONRⁱRⁱⁱ, —SO₂R, —SR, —R, —SiRⁱRⁱⁱRⁱⁱⁱ, —PORⁱRⁱⁱ, —PSRⁱRⁱⁱ, —PO(ORⁱ)(ORⁱⁱ); protective groups Prot¹ and Prot² or Prot¹ and Prot³ may be also connected to each other especially according to following general structure: —CRⁱRⁱⁱ—, —CO—, —COCO—, —CS—, —C(═NR)—, —COCRⁱRⁱⁱCO—, —CO—R—CO—, [—CRⁱRⁱⁱCRⁱRⁱⁱ]₂SO, [—CRⁱRⁱⁱCRⁱRⁱⁱ]₂SO₂, [—CRⁱRⁱⁱCRⁱRⁱⁱ]₂P(O)OR, [—CRⁱRⁱⁱCRⁱRⁱⁱ]₂NR, —PO(OR)—, —SiRⁱRⁱⁱ—, —SnRⁱRⁱⁱ— wherein Rⁱ⁻ⁱⁱⁱ are groups of the same type as R. Prot¹⁻³ is for example methanesulphonyl, 4-toluenesulphonyl, trifluoromethanesulphonyl, nitrobenzenesulphonyl, benzenesulphonyl, naphthalenesulphonyl, formyl, acetyl, benzoyl, phthaloyl, trifluoroacetyl, tert.-butoxycarbonyl (Boc), 9H-fluoren-9-yl-methoxycarbonyl (Fmoc), benzyloxycarbonyl (Z), ethoxycarbonyl (Eoc), methoxycarbonyl (Meoc), methoxybenzylcarbonyl (Moz), trityl, benzyl, benzhydryl, 4,4′-dimethoxytrityl, 4-methoxybenzoyl, ethandioyl, propandioyl, carbonyl, thiocarbonyl etc.
by reaction (e.g. condensation) with precursors or their mixture of structure: 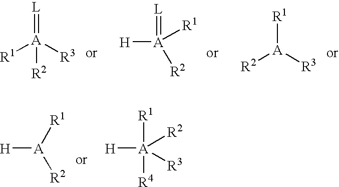
wherein R³ and R⁴ are groups of the same type as R¹⁻². Especially can be used: alkylphosphinic acid, arylphosphinic acid, trialkylphosphites, triarylphosphites, trialkylphosphines, triarylphosphines, dialkylphosphinates, diarylphosphinates, alkylarylphosphinates, dialkylarylphosphites, alkyldiarylphosphites, phosphinic acid, alkylarsenic(III) acid, arylarsenic(III) acid, trialkylarsenic(III), triarylarsenic(III), etc.
under conditions of general nucleofilic substitution: especially under conditions of phase-transfer catalysis, in aprotic polar solvents or its mixtures (as dimethylformamide or dimethylacetamide acetonitrile, dimethylsulphoxide or sulpholane or hexamethylphosphortriamide), in micellar medium, in solidphase (for example bonded N₄G⁻ on anex), with or without microwave irradiation, with or without ultrasonic irradiation, under conditions of high pressure (for example in autoclave), in aqueous phase in presence of pH-buffer, in milieu of water-free solvents with or without presence of base (for example: amines, aldimines, anex, carbonates, fluorides, thioethers), enzymatic catalysis, in presence of dehydrating agent or agent reacting with protogenic product reaction or in presence of Lewis acid as catalysators (for example ZnCl₂, BF₃.Et₂O, SiCl₄) etc.

7. Method of preparation or synthesis of ligands of the general formula (I) as defined in claim 1 from unprotected ligands N₄H₄/N₄H₃⁻ of structure: 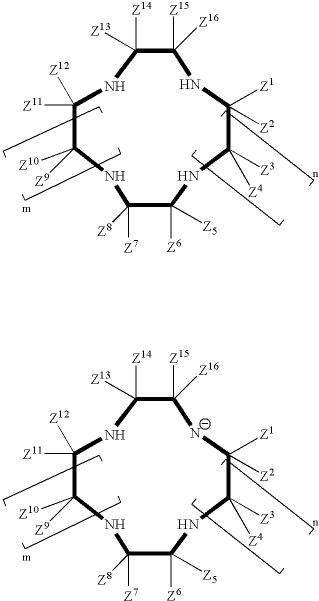
by condensation with Subst-(Q)ₚA(L)(R¹)(R²) of structure: 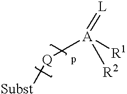
wherein Subst is general leaving group, of structure: —OR, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱⁱ)(SO₂Rⁱⁱ), —NSO₂R, -halogene, —NRⁱNRⁱⁱRⁱⁱⁱ, —SR, —SO₃H or —SO₂Y¹⁻³, wherein Rⁱ⁻ⁱⁱⁱ are groups of the same type as R. (Subst is for example: hydroxyle, alcoxyl, aryloxyl, alkyl amine, N,N-dialkylamine, N-alkylamine, arylsulfonyloxy, tosyloxy, mesyloxy, triflyloxy, acetoxy, benzoyloxy, N-benztriazolyl, trialkylsilyloxy, hydrazine and N-substituted hydrazine, benzyloxycarbonyloxy, tert.-butyloxycarbonyloxy, 1-imidazolyl, succinimidyloxy, N-succinimidyl, N-phthalimidyl, N-phthalimidyloxy, arylthio, thiole, S-alkylthiole etc.), or group which generates in situ or on introduced functional group (Q)ₚA(L)(R¹)(R²) with or without isolation cation or partial positive charge with capability to reaction (photochemically, thermically or electrochemically cleavable groups)
under conditions of general nucleofilic substitution: especially under conditions of high dilution, under conditions of phase-transfer catalysis, in aprotic polar solvents or its mixtures (as dimethylformamide or dimethylacetamide acetonitrile, dimethylsulphoxide or sulpholane or hexamethylphosphortriamide), in micellar medium, in solidphase (for example bonded N₄G⁻ on anex), with or without microwave irradiation, with or without ultrasonic irradiation, under conditions of high pressure (for example in autoclave), in aqueous phase in presence of pH-buffer, under conditions of N-alkylation in presence cations of metals (e.g. calcium, magnesium, cooper, nickel, iron, lithium) or organic cations (e.g. tetramethylammonium), in millieu of water-free solvents with or without presence of base (for example: amines, aldimines, carbonates, fluorides, thioethers), in general two or multiphase systems etc.
with or without separation of monosubstituted ligand N₄H₃(Q)ₚA(L)(R¹)(R²) from reaction mixture. Separation can be carried out by chromatography techniques as: HPLC or LC, ionex chromatography or on ionex column generally, preparative TLC, paper chromatography, gel chromatography, etc., especially by gradient elution on HPLC or LC, or by extraction from water solutions to water immiscible solvents or its mixtures (e.g. dichloromethane, chloroform, ethyl acetate, 1-butylacetate, chlorobenzene, hexane) continuously or discontinuously, at high temperatures or under cooling (e.g. by cryogenic techniques). Separation also can be carried out by precipitation or coagulation, by freezing out, by sublimation out of reactant, by continuous extracting out monosubstituted ligand N₄H₃(Q)ₚA(L)(R¹)(R²) or by-products, etc.
and possible next reaction
a) with 3 moles of reactant Subst-(X^{t})C(Y^{t})(W^{t}) (t is 1 or 2 or 3) or independently by step reactions with Subst-(X¹)C(Y¹)(W¹), Subst-(X²)C(Y²)(W²) and Subst-(X²)C(Y²)(W²)) of structure: 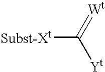
wherein Subst is general leaving group, of structure: —OR, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, wherein Rⁱ⁻ⁱⁱⁱ are groups of the same type as R. (Subst is for example: hydroxyle, alcoxyl, aryloxyl, alkyl amine, N,N-dialkylamine, N-alkylamine, arylsulfonyloxy, tosyloxy, mesyloxy, triflyloxy, acetoxy, benzoyloxy, N-benztriazolyl, trialkylsilyloxy, hydrazine and N-substituted hydrazine, benzyloxycarbonyloxy, ter.-butyloxycarbonyloxy, 1-imidazolyl, succinimidyloxy, N-succinimidyl, N-phthalimidyl, N-phthalimidyloxy, arylthio, thiole, S-alkylthiole etc.), or group which generates in situ or on introduced functional group (X^{t})C(Y^{t})(W^{t}) with or without isolation cation or partial positive charge with capability to reaction (photochemically, thermically or electrochemically cleavable groups)
or by reaction with an intermediate Subst-(CZ¹Z²)ₙCN or by reaction with an intermediate generated in situ by general reaction of HCN and Z¹-C(=L)-Z² and by next hydrolysis with or without isolation of structure: 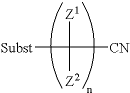
wherein n is 1 or 2,
or by reaction with intermediates Subst-(CZ¹Z²)ₙC(═W^{t-t″}Rⁱ⁻ⁱⁱⁱ)₃ or Subst-(CZ¹Z²)ₙC(W^{t-t′}Rⁱ⁻ⁱⁱ)₂R or Subst-(CZ¹Z²)ₙC(═W¹⁻³)R or —(CZ¹Z²)(CZ³Z⁴)W¹⁻³— and by next hydrolytic, oxidative, reduction cleavage of structure: 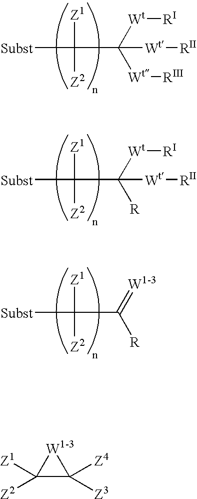
wherein Rⁱ⁻ⁱⁱⁱ is independently group of the same type as R¹⁻², t, t′ and t″ is 1 or 2 or 3, n is 1 or 2.
b) by addition on an intermediate Q-C(Y^{t})(W^{t}) (t is 1 or 2 or 3) or independently by step-reaction with Q-C(Y¹)(W¹), Q-C(Y²)(W²) and Q-C(Y³)(W³) of structure: 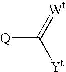
wherein anywhere on Q is double or multiple bond with capability to add intermediates under conditions of general addition: especially carried out under high-pressure (for example in autoclave), microwave irradiation, under reflux in high boiling solvents, in micellar systems, in solid phase, under cryogenic conditions, under phase transfer catalysis, etc.
Double or multiple bonds can be generated in situ with or without isolation by general elimination methods from (Q-(XY)ₙ)—(X^{t})C(Y^{t})(W^{t}) by elimination of XY, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc.
n is from 1 to 4.
Double or multiple bonds on (Q)-(X^{t})C(Y^{t})(W^{t}) can be generated also from other substituents, which constitute (Q)-(X^{t})C(Y^{t})(W^{t}) under conditions of irradiation (include thermic) or electrochemical reactions, e.g. tetrazenes, cyclic azides, triazenes, dixandiones etc.
or by addition with an intermediate Q-CN and by next hydrolysis with or without isolation of structure:
Q-CN
Double or multiple bonds can be generated in situ with or without isolation by general elimination of XY from (Q-(XY)ₙ)—CN, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc.
n is from 1 to 4.
Double or multiple bonds on Q-CN can be generated also from other substituents, which constitute Q-CN under conditions of irradiation (include thermic) or electrochemical reactions, e.g. tetrazenes, cyclic azides, triazenes, dixandiones etc.
or by addition on intermediates Q-C(W^{t-t″}Rⁱ⁻ⁱⁱⁱ)₃ or Q-C(W^{t-t′}Rⁱ⁻ⁱⁱ)₂R or Q-C(═W¹⁻³)R and by next hydrolytic, oxidative, reduction cleavage of structure: 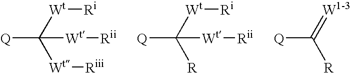
wherein Rⁱ⁻ⁱⁱⁱ is independently group of the same type as R¹⁻², t, t′ and t″ is 1 or 2 or 3.
Double or multiple bonds can be generated in situ with or without isolation by general elimination of XY from (Q-(XY)ₙ)—C(W^{t-t″}Rⁱ⁻ⁱⁱⁱ)₃ or (Q-(XY)ₙ)—C(W^{t-t′}Rⁱ⁻ⁱⁱ)₂R or (Q-(XY)ₙ)—C(═W¹⁻³)R, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc.
n is from 1 to 4.
Double or multiple bonds on Q-C(W^{t-t″}Rⁱ⁻ⁱⁱⁱ)₃ or Q-C(W^{t-t′}Rⁱ⁻ⁱⁱ)₂R or Q-C(═W¹⁻³)R can be generated also from other substituents, which constitute Q-C(W^{t-t″}Rⁱ⁻ⁱⁱⁱ)₃ or Q-C(W^{t-t′}Rⁱ⁻ⁱⁱ)₂R or Q-C(═W¹⁻³)R under conditions of irradiation (include thermic) or electrochemical reactions, e.g. tetrazenes, cyclic azides, triazenes, dixandiones etc.
c) or by addition and subsequent reduction on an intermediate R^{t}—C(═W^{t′}) (CZ¹Z²)_{f}C(Y^{t})(W^{t}) or independently by step-reaction and subsequent reduction or in situ reduction on R—C(═W^{t′})(CZ¹Z²)_{f}C(1)(W¹), R—C(═W^{t′}) (CZ³Z⁴)_{f}C(Y²)(W²) and R—C(═W^{t′})(CZ⁵Z⁶)_{f}C(Y³)(W³) of structure: 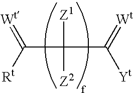
wherein R^{t} is independently group of the same type as R¹⁻², f is 0 or 1, t and t′ is 1 or 2 or 3.
or by addition on intermediates R^{t}—C(═W^{t′})(CZ¹Z²)_{f}C(W^{t-t′}Rⁱ⁻ⁱⁱⁱ)₃ or R^{t}—C (═W^{t′})(CZ¹Z²)_{f}C(W^{t-t′}Rⁱ⁻ⁱⁱ)₂R or R^{t}—C(═W^{t′})(CZ¹Z²)_{f}C(═W¹⁻³)R and by subsequent reduction or in situ reduction or by subsequent hydrolytic, oxidative, reduction cleavage of structure: 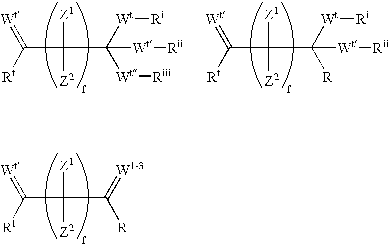
wherein R^{t} is independently group of the same type as R¹⁻², Rⁱ⁻ⁱⁱⁱ is independently group of the same type as R¹⁻², t, t′ and t″ is 1 or 2 or 3, f is 0 or 1.

8. Method of preparation or synthesis of ligands of the general formula (1) as defined in claim 1 from unprotected ligands N₄H₄/N₄H₃⁻ of structure: 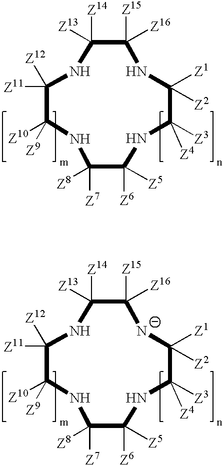
by addition on (Q)A(L)(R¹)(R²) of structure: 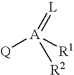
wherein anywhere on Q is double or multiple bond with capability to add intermediates (N₄GH or N₄G⁻) under conditions of general addition: especially carried out under high-pressure (for example in autoclave), microwave irradiation, under reflux in high boiling solvents, in micellar systems, in solid phase, under cryogenic conditions, under phase transfer catalysis, etc.
Double or multiple bonds can be generated in situ with or without isolation by general elimination methods from (Q-(XY)ₙ)A(L)(R¹)(R²) by elimination of XY, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc. n is from 1 to 12.
Double or multiple bonds on (Q)A(L)(R¹)(R²) can be generated also from other substituents, which constitute (Q)A(L)(R¹)(R²) under conditions of irradiation (include thermic) or electrochemical reactions, e.g. tetrazenes, cyclic azides, triazenes, dixandiones etc.
with or without separation of monosubstituted ligand N₄H₄ from reaction mixture. Separation can be carried out by chromatography techniques as: HPLC or LC, Ionex chromatography or on ionex column generally, preparative TLC, paper chromatography, gel chromatography, etc., especially by gradient elution on HPLC or LC, or by extraction from water solutions to water immiscible solvents or its mixtures (e.g. dichloromethane, chloroform, ethyl acetate, 1-butylacetate, chlorobenzene, hexane) continuously or discontinuously, at high temperatures or under cooling (e.g. by cryogenic techniques). Separation also can be carried out by precipitation or coagulation, by freezing out, by sublimation out of reactant etc.
or by addition and subsequent reduction (or in situ reduction) on an intermediate R—C(═W¹⁻³)(Q)ₚₚA(L)(R¹)(R²) of structure: 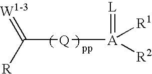
wherein pp is from 0 to 9.
and possible next reaction
a) with 3 moles of reactant Subst-(X^{t})C(Y^{t})(W^{t}) (t is 1 or 2 or 3) or independently by step reactions with Subst-(X¹)C(Y¹)(W¹), Subst-(X²)C(Y²)(W²) and Subst-(X²)C(Y²)(W²) of structure: 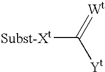
wherein Subst is general leaving group, of structure: —OR, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, wherein Rⁱ⁻ⁱⁱⁱ are groups of the same type as R. (Subst is for example: hydroxyle, alcoxyl, aryloxyl, alkyl amine, N,N-dialkylamine, N-alkylamine, arylsulfonyloxy, tosyloxy, mesyloxy, triflyloxy, acetoxy, benzoyloxy, N-benztriazolyl, trialkylsilyloxy, hydrazine and N-substituted hydrazine, benzyloxycarbonyloxy, tert.-butyloxycarbonyloxy, 1-imidazolyl, succinimidyloxy, N-succinimidyl, N-phthalimidyl, N-phthalimidyloxy, arylthio, thiole, S-alkylthiole etc.), or group which generates in situ or on introduced functional group (X^{t})C(Y^{t})(W^{t}) with or without isolation cation or partial positive charge with capability to reaction (photochemically, thermically or electrochemically cleavable groups)
or by reaction with an intermediate Subst-(CZ¹Z²)ₙCN or by reaction with an intermediate generated in situ by general reaction of HCN and Z¹-C (=L)-Z² and by next hydrolysis with or without isolation of structure: 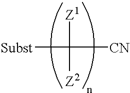
wherein n is 1 or 2,
or by reaction with intermediates Subst-(CZ¹Z²)ₙC(W^{t-t″}Rⁱ⁻ⁱⁱⁱ)₃ or Subst-(CZ¹Z²)ₙC(W^{t-t′}Rⁱ⁻ⁱⁱ)₂R or Subst-(CZ¹Z²)ₙC(═W¹⁻³)R or —(CZ¹Z²)(CZ³Z⁴)W¹⁻³— and by next hydrolytic, oxidative, reduction cleavage of structure: 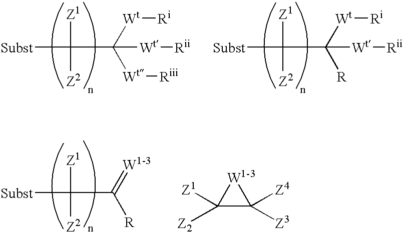
wherein Rⁱ⁻ⁱⁱⁱ is independently group of the same type as R¹⁻², t, t′ and t″ is 1 or 2 or 3, n is 1 or 2.
b) by addition on an intermediate Q-C(Y^{t})(W^{t}) (t is 1 or 2 or 3) or independently by step-reaction with Q-C(Y¹)(W¹), Q-C(Y²)(W²) and Q-C(Y³)(W³) of structure: 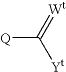
wherein anywhere on Q is double or multiple bond with capability to add intermediates under conditions of general addition: especially carried out under high-pressure (for example in autoclave), microwave irradiation, under reflux in high boiling solvents, in micellar systems, in solid phase, under cryogenic conditions, under phase transfer catalysis, etc.
Double or multiple bonds can be generated in situ with or without isolation by general elimination methods from (Q-(XY)ₙ)—(X^{t})C(Y^{t})(W^{t}) by elimination of XY, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc.
n is from 1 to 4.
Double or multiple bonds on (Q)-(X^{t})C(Y^{t})(W^{t}) can be generated also from other substituents, which constitute (Q)-(X^{t})C(Y^{t})(W^{t}) under conditions of irradiation (include thermic) or electrochemical reactions, e.g. tetrazenes, cyclic azides, triazenes, dixandiones etc.
or by addition with an intermediate Q-CN and by next hydrolysis with or without isolation of structure:
Q-CN
Double or multiple bonds can be generated in situ with or without isolation by general elimination of XY from (Q-(XY))—CN, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc.
n is from 1 to 4.
Double or multiple bonds on O—CN can be generated also from other substituents, which constitute Q-CN under conditions of irradiation (include thermic) or electrochemical reactions, e.g. tetrazenes, cyclic azides, triazenes, dixandiones etc.
or by addition on intermediates Q-C(W^{t-t″}Rⁱ⁻ⁱⁱⁱ)₃ or Q-C(W^{t-t′}Rⁱ⁻ⁱⁱ)₂R or Q-C(═W¹⁻³)R and by next hydrolytic, oxidative, reduction cleavage of structure: 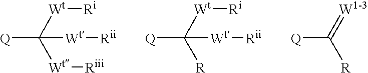
wherein Rⁱ⁻ⁱⁱⁱ is independently group of the same type as R¹⁻², t, t′ and t″ is 1 or 2 or 3.
Double or multiple bonds can be generated in situ with or without isolation by general elimination of XY from (Q-(XY)ₙ) C(W^{t-t″}Rⁱ⁻ⁱⁱⁱ)₃ or (Q-(XY)ₙ)—C(W^{t-t′}Rⁱ⁻ⁱⁱ)₂R or (Q-(XY)ₙ)—C(═W¹⁻³)R, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc.
n is from 1 to 4.
Double or multiple bonds on Q-C(W^{t-t″}Rⁱ⁻ⁱⁱⁱ)₃ or Q-C(W^{t-t′}Rⁱ⁻ⁱⁱ)₂R or Q-C(═W¹⁻³)R can be generated also from other substituents, which constitute Q-C(W^{t-t″}Rⁱ⁻ⁱⁱⁱ)₃ or Q-C(W^{t-t′}Rⁱ⁻ⁱⁱ)₂R or Q-C(═W¹⁻³)R under conditions of irradiation (include thermic) or electrochemical reactions, e.g. tetrazenes, cyclic azides, triazenes, dixandiones etc.
c) or by addition and subsequent reduction on an intermediate R^{t}—C(═W^{t′})(CZ¹Z²)_{f}C(Y^{t})(W^{t}) or independently by step-reaction and subsequent reduction or in situ reduction on R—C(═W^{t′})(CZ¹Z²)_{f}C(Y¹)(W¹), R—C(═W^{t′})(CZ³Z⁴)_{f}C(Y²)(W²) and R—C(═W^{t′})(CZ⁵Z⁶)_{f}C)(Y³)(W³) of structure: 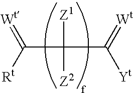
wherein R^{t} is independently group of the same type as R¹⁻², f is 0 or 1, t and t′ is 1 or 2 or 3.
or by addition on intermediates R^{t}—C(═W^{t′})(CZ¹Z²)_{f}C(W^{t-t″}Rⁱ⁻ⁱⁱⁱ)₃ or R^{t}—C(═W^{t′})(CZ¹Z²)_{f}C(W^{t-t′}Rⁱ⁻ⁱⁱ)₂R or R^{t}—C(═W^{t′})(CZ¹Z²)_{f}C(═W¹⁻³)R and by subsequent reduction or in situ reduction or by subsequent hydrolytic, oxidative, reduction cleavage of structure: 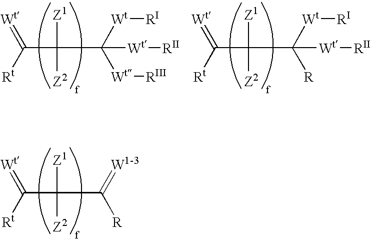
wherein R^{t} is independently group of the same type as R¹⁻², Rⁱ⁻ⁱⁱⁱ is independently group of the same type as R¹⁻², t, t′ and t″ is 1 or 2 or 3, f is 0 or 1.

9. Method of preparation or synthesis of ligands of the general formula (1) as defined in claim 1 from monoprotected ligand of structure: 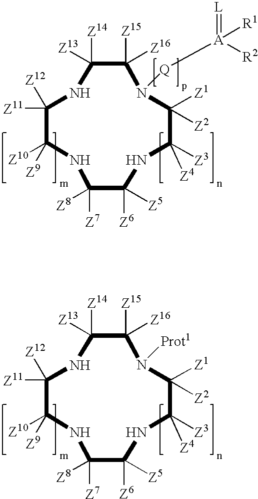
wherein Prot¹ is protective group (or electron pair with negative charge) especially of general structure: —CHO, —COR, —COOR, —CONRⁱRⁱⁱ, —SO₂R, —SR, —R, —SiRⁱRⁱⁱRⁱⁱⁱ, —PORⁱRⁱⁱ, —PSRⁱRⁱⁱ, —PO(ORⁱ)(ORⁱⁱ); wherein Rⁱ⁻ⁱⁱⁱ are groups of the same type as R. Prot¹ is for example methanesulphonyl, 4-toluenesulphonyl, trifluoromethanesulphonyl, nitrobenzenesulphonyl, benzenesulphonyl, naphthalenesulphonyl, formyl, acetyl, benzoyl, phthaloyl, trifluoroacetyl, tert.-butoxycarbonyl (Boc), 9H-fluoren-9-yl-methoxycarbonyl (Fmoc), benzyloxycarbonyl (Z), ethoxycarbonyl (Eoc), methoxycarbonyl (Meoc), methoxybenzylcarbonyl (Moz), trityl, benzyl, benzhydryl, 4,4-dimethoxytrityl, 4-methoxybenzoyl, ethandioyl, propandioyl, carbonyl, thiocarbonyl etc.
by reaction (e.g. nuceleofilic substitution, addition)
a) with 3 moles of reactant Subst-(X^{t})C(Y^{t})(W^{t}) (t is 1 or 2 or 3) or independently by step reactions with Subst-(X¹)C(Y¹)(W¹), Subst-(X²)C(Y²)(W²) and Subst-(X²)C(Y²)(W²)) of structure: 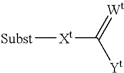
wherein Subst is general leaving group, of structure: —OR, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, wherein Rⁱ⁻ⁱⁱⁱ are groups of the same type as R. (Subst is for example: hydroxyle, alcoxyl, aryloxyl, alkyl amine, N,N-dialkylamine, N-alkylamine, arylsulfonyloxy, tosyloxy, mesyloxy, triflyloxy, acetoxy, benzoyloxy, N-benztriazolyl, trialkylsilyloxy, hydrazine and N-substituted hydrazine, benzyloxycarbonyloxy, tert.-butyloxycarbonyloxy, 1-imidazolyl, succinimidyloxy, N-succinimidyl, N-phthalimidyl, N-phthalimidyloxy, arylthio, thiole, S-alkylthiole etc.), or group which generates in situ or on introduced functional group (X^{t})C(Y^{t})(W^{t}) with or without isolation cation or partial positive charge with capability to reaction (photochemically, thermically or electrochemically cleavable groups)
or by reaction with an intermediate Subst-(CZ¹Z²)ₙCN or by reaction with an intermediate generated in situ by general reaction of HCN and Z¹-C(=L)-Z² and by next hydrolysis with or without isolation of structure: 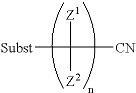
wherein n is 1 or 2,
or by reaction with intermediates Subst-(CZ¹Z²)ₙC(W^{t-t″}Rⁱ⁻ⁱⁱⁱ)₃ or Subst-(CZ¹Z²)ₙC(W^{t-t′}Rⁱ⁻ⁱⁱ)₂R or Subst-(CZ¹Z²)ₙC(═W¹⁻³)R or —(CZ¹Z²)(CZ³Z⁴)W¹⁻³— and by next hydrolytic, oxidative, reduction cleavage of structure: 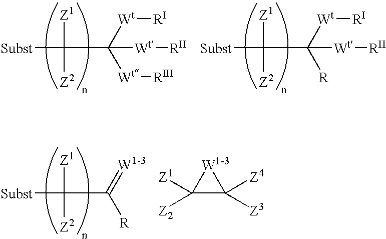
wherein Rⁱ⁻ⁱⁱⁱ is independently group of the same type as R¹⁻², t, t′ and t″ is 1 or 2 or 3, n is 1 or 2.
b) by addition on an intermediate Q-C(Y^{t})(W^{t}) (t is 1 or 2 or 3) or independently by step-reaction with Q-C(Y¹)(W¹), Q-C(Y²)(W²) and Q-C(Y³)(W³) of structure: 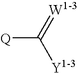
wherein anywhere on Q is double or multiple bond with capability to add intermediates under conditions of general addition: especially carried out under high-pressure (for example in autoclave), microwave irradiation, under reflux in high boiling solvents, in micellar systems, in solid phase, under cryogenic conditions, under phase transfer catalysis, etc.
Double or multiple bonds can be generated in situ with or without isolation by general elimination methods from (Q-(XY)ₙ)—(X^{t})C(Y^{t})(W^{t}) by elimination of XY, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc.
n is from 1 to 4.
Double or multiple bonds on (Q)-(X^{t})C(Y^{t})(W^{t}) can be generated also from other substituents, which constitute (Q)-(X^{t})C(Y^{t})(W^{t}) under conditions of irradiation (include thermic) or electrochemical reactions, e.g. tetrazenes, cyclic azides, triazenes, dixandiones etc.
or by addition with an intermediate Q-CN and by next hydrolysis with or without isolation of structure:
Q-CN
Double or multiple bonds can be generated in situ with or without isolation by general elimination of XY from (Q-(XY)ₙ)—CN, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc.
n is from 1 to 4.
Double or multiple bonds on Q-CN can be generated also from other substituents, which constitute Q-CN under conditions of irradiation (include thermic) or electrochemical reactions, e.g. tetrazenes, cyclic azides, triazenes, dixandiones etc.
or by addition on intermediates Q-C(W^{t-t″}Rⁱ⁻ⁱⁱⁱ)₃ or Q-C(W^{t-t′}Rⁱ⁻ⁱⁱ)₂R or Q-C(═W¹⁻³)R and by next hydrolytic, oxidative, reduction cleavage of structure: 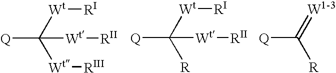
wherein Rⁱ⁻ⁱⁱⁱ is independently group of the same type as R¹⁻², t, t′ and t″ is 1 or 2 or 3.
Double or multiple bonds can be generated in situ with or without isolation by general elimination of XY from (Q-(XY)ₙ)—C(W^{t-t″}Rⁱ⁻ⁱⁱⁱ)₃ or (Q-(XY)ₙ)—(W^{t-t′}Rⁱ⁻ⁱⁱ)₂R or (Q-(XY)ₙ)—C(═W¹⁻³)R, wherein:
XY is thermodynamically stable compound capable to elimination, especially: nitrogen, sulphur, ammonia, water, hydrogen sulphide, hydrogen halogenide, metal halogenide, hydrogen or metal alkyl- or arylcarboxylates, hydrogen or metal sulphonate or substituted sulphonate, etc.
n is from 1 to 4.
Double or multiple bonds on Q-C(W^{t-t″}Rⁱ⁻ⁱⁱⁱ)₃ or Q-C(W^{t-t′}Rⁱ⁻ⁱⁱ)₂R or Q-C(═W¹⁻³)R can be generated also from other substituents, which constitute Q-C(W^{t-t″}Rⁱ⁻ⁱⁱⁱ)₃ or Q-C(W^{t-t′}Rⁱ⁻ⁱⁱ)₂R or Q-C(═W¹⁻³)R under conditions of irradiation (include thermic) or electrochemical reactions, e.g. tetrazenes, cyclic azides, triazenes, dixandiones etc.
c) or by addition and subsequent reduction on an intermediate R^{t}—C(═W^{t′})(CZ¹Z²)_{f}C(Y^{t})(W^{t}) or independently by step-reaction and subsequent reduction or in situ reduction on R—C(═W^{t′})(CZ¹Z²)_{f}C(Y¹)(W¹), R—C(═W^{t′})(C³Z⁴)_{f}C(Y²)(W²) and R—C(═W^{t′})(CZ⁵Z⁶)_{f}C(Y³)(W³) of structure: 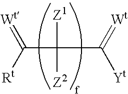
wherein R^{t} is independently group of the same type as R¹⁻², f is 0 or 1, t and t′ is 1 or 2 or 3.
or by addition on intermediates R^{t}—C(═W^{t′})(CZ¹Z²)_{f}C(W^{t-t″}Rⁱ⁻ⁱⁱⁱ)₃ or R^{t}—C(═W^{t′})(CZ¹Z²)_{f}C(W^{t-t′}Rⁱ⁻ⁱⁱ)₂R or R^{t}—C(═W^{t′})(CZ¹Z²)_{f}C(═W¹⁻³)R and by subsequent reduction or in situ reduction or by subsequent hydrolytic, oxidative, reduction cleavage of structure: 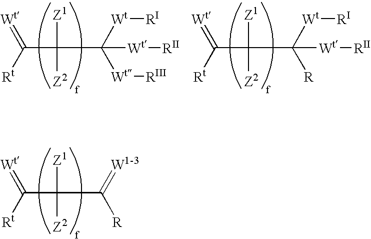
wherein R^{t} is independently group of the same type as R¹⁻², Rⁱ⁻ⁱⁱⁱ is independently group of the same type as R¹⁻², t, t′ and t″ is 1 or 2 or 3, f is 0 or 1.

10. Method of preparation or synthesis of ligands of the general formula (1) as defined in claim 1 from tetraprotected ligands N₄(CR¹CRⁱⁱ), N₄M, N₄Pg¹Pg², N₄Pg¹Prot¹Prot² of structure: 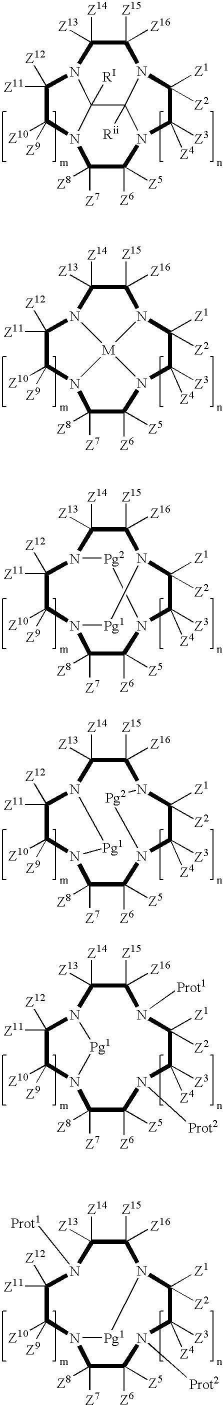
wherein Rⁱ and Rⁱⁱ are groups of the same type as R; M is PR, P—SR, P-halogen, P—OR, silicon, carbon;
Pg¹⁻² is independently protective group especially of structure: CRⁱRⁱⁱ, SiRⁱRⁱⁱ, SnRⁱRⁱⁱ, CO, CS, C(═NR), PO(OR), PS(OR), PO(R), PS(R);
Prot¹ and Prot² is independently protective group (or electron pair with negative charge) especially of general structure: —CHO, —COR, —COOR, —CONRⁱRⁱⁱ, —SO₂R, —SR, —R, —SiRⁱRⁱⁱRⁱⁱⁱ, —PORⁱRⁱⁱ, —PSRⁱRⁱⁱ, —PO(ORⁱ)(ORⁱⁱ); protective groups Prot¹ and Prot² may be also connected to each other especially according to following general structure: —CRⁱRⁱⁱ—, —CO—, —COCO—, —CS—, —C(═NR)—, —COCRⁱRⁱⁱCO—, —CO—R—CO—, [—CRⁱRⁱⁱCRⁱRⁱⁱ]₂SO, [—CRⁱRⁱⁱCRⁱRⁱⁱ]₂SO₂, [—CRⁱRⁱⁱCRⁱRⁱⁱ]₂P(O)OR, [—CRⁱRⁱⁱCRⁱRⁱⁱ]₂NR, —PO(OR)—, —SiRⁱRⁱⁱ—, —SnRⁱRⁱⁱ— wherein Rⁱ⁻ⁱⁱⁱ are groups of the same type as R. Prot¹⁻² is for example methanesulphonyl, 4-toluenesulphonyl, trifluoromethanesulphonyl, nitrobenzenesulphonyl, benzenesulphonyl, naphthalenesulphonyl, formyl, acetyl, benzoyl, phthaloyl, trifluoroacetyl, tert.-butoxycarbonyl (Boc), 9H-fluoren-9-yl-methoxycarbonyl (Fmoc), benzyloxycarbonyl (Z), ethoxycarbonyl (Eoc), methoxycarbonyl (Meoc), methoxybenzylcarbonyl (Moz), trityl, benzyl, benzhydryl, 4,4′-dimethoxytrityl, 4-methoxybenzoyl, ethandioyl, propandioyl, carbonyl, thiocarbonyl etc.
by condensation with Subst-(Q)ₚA(L)(R¹)(R²) of structure: 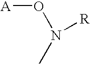
wherein Subst is general leaving group, of structure: —OR, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, (NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³ wherein Rⁱ⁻ⁱⁱⁱ are groups of the same type as R. (Subst is for example: hydroxyle, alcoxyl, aryloxy), alkyl amine, N,N-dialkylamine, N-alkylamine, arylsulfonyloxy, tosyloxy, mesyloxy, triflyloxy, acetoxy, benzoyloxy, N-benztriazolyl, trialkylsilyloxy, hydrazine and N-substituted hydrazine, benzyloxycarbonyloxy, tert.-butyloxycarbonyloxy 1-imidazolyl, succinimidyloxy, N-succinimidyl, N-phthalimidyl, N-phthalimidyloxy, arylthio, thiole, S-alkylthiole etc.), or group which generates in situ or on introduced functional group (Q)ₚA(L)(R¹)(R²) with or without isolation cation or partial positive charge with capability to reaction (photochemically, thermically or electrochemically cleavable groups)
under conditions of general nucleofilic substitution: especially under conditions of phase-transfer catalysis, in aprotic polar solvents or its mixtures (as dimethylformamide or dimethylacetamide acetonitrile, dimethylsulphoxide or sulpholane or hexamethylphosphortriamide), in micellar medium, in solidphase (for example bonded N₄G⁻ on anex), with or without microwave irradiation, with or without ultrasonic irradiation, under conditions of high pressure (for example in autoclave), in aqueous phase in presence of pH-buffer, in milieu of water-free solvents with or without presence of base (for example: amines, aldimines, anex, carbonates, fluorides, thioethers), enzymatic catalysis, in presence of dehydrating agent or agent reacting with protogenic product reaction or in presence of Lewis acid as catalysators (for example ZnCl₂, BF₃.Et₂O, SiCl₄) etc.
with or without separation of quaternary monosubstituted ligand from reaction mixture or pentacoordinated N₄M-(Q)ₚA(L)(R¹)(R²) phosphorane.
and by next possible partially or full cleaving of >CRⁱRⁱⁱ and >CRⁱⁱⁱCR^{iv}< bridges or protective groups Prot¹⁻² or Pg¹⁻². Both the steps can be solved as one-step reaction or separately and also by isomerisation of pentacoordinated N₄M-(Q)ₚA(L)(R¹)(R²) phosphorane to N₄G(Q)ₚA(L)(R¹)(R²) all in the conditions described by method in i).

11. Method of preparation or synthesis of ligands of the general formula (1) as defined in claim 1 from uncyclic intermediate of structure: 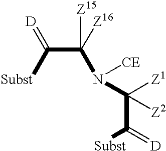
wherein CE is equivalent of (Q)ₚA(L)(R¹)(R²) or Prot¹⁻³ from points i) to viii) from description of ligands; Subst is equivalent leaving group as Subst from points vii) or viii) from description of ligands; D is e.g. oxygene, hydrogen pair, N-substituted or unsubstituted nitrogene, sulphur
by reaction with derivate of structure: 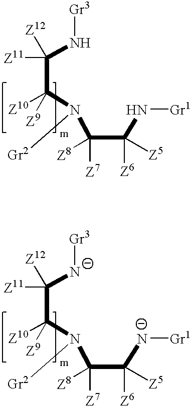
wherein Gr¹⁻³ is group independently equivalent with (X^{t})C(Y^{t})(W^{t}) (t is 1 or 2 or 3) or Prot¹⁻³ from points i) to viii) from description of ligands
under conditions of high dilution, template synthesis, reaction on solid phase, phase transfer catalysis, in aprotic polar solvents, with or without microwave irradiation, with or without presence of ultrason.

12. Method of preparation or synthesis of ligands of the general formula (1) as defined in claim 1 from uncyclic intermediate of structure: 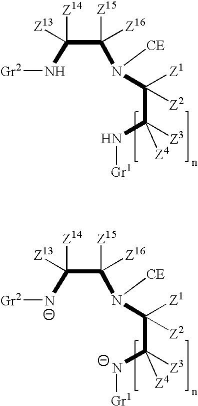
wherein CE is equivalent of (Q)_{pA}(L)(R¹)(R²) or Prot¹⁻³ from points i) to viii) from description of ligands; Gr¹⁻² is group independently equivalent with (X^{t})C(Y^{t})(W^{t}) (t is 1 or 2 or 3) or Prot¹⁻³ from points i) to viii) from description of ligands
by reaction with derivate of structure: 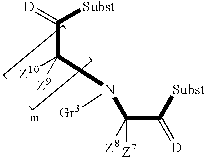
wherein Subst is equivalent leaving group as Subst from points vii) or viii) from description of ligands; Gr³ is group independently equivalent with (X^{t})C(Y^{t})(W^{t}) (t is 1 or 2 or 3) or Prot¹⁻³ from points i) to viii) from description of ligands; D is e.g. oxygene, hydrogen pair, N-substituted or unsubstituted nitrogene, sulphur
under conditions of high dilution, template synthesis, reaction on solid phase, phase transfer catalysis, in aprotic polar solvents, with or without microwave irradiation, with or without presence of ultrasonic.

13. Method of preparation or synthesis of ligands of the general formula (1) as defined in claim 1 from uncyclic intermediate of structure: 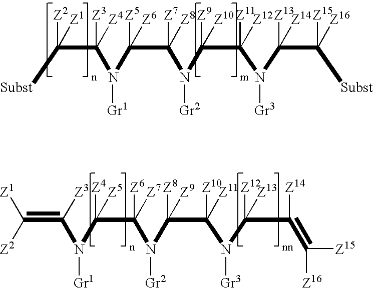
wherein Gr¹⁻³ is group independently equivalent with (X^{t})C(Y^{t})(W^{t}) (t is 1 or 2 or 3) or Prot¹⁻³ from points i) to viii) from description of ligands, Subst is equivalent leaving group as Subst from points vii) or viii) from description of ligands; Gr³ is group independently equivalent with (X^{t})C(Y^{t})(W^{t}) (t is 1 or 2 or 3) or Prot¹⁻³ from points i) to viii) from description of ligands; n, m is independently 1 or 2, nn is 0 or 1;
by reaction with derivate of structure:
H₂N—CE
wherein CE is equivalent of (Q)A(L)(R¹)(R²) or Prot¹⁻³ from points i) to viii) from description of ligands;
under conditions of high dilution, template synthesis, reaction on solid phase, phase transfer catalysis, in aprotic polar solvents, with or without microwave irradiation, with or without presence of ultrasonic.

14. Method of preparation or synthesis of ligands of the general formula (1) as defined in claim 1 from uncyclic intermediate of structure: 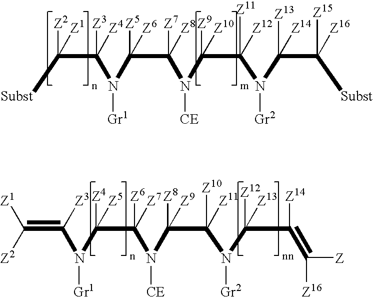
wherein CE is equivalent of (Q)A(L)(R¹)(R²) from points i) to viii) from description of ligands; Gr¹⁻² is group independently equivalent with (X^{t})C(Y^{t})(W^{t}) (t is 1 or 2 or 3) or Prot¹⁻³ from points i) to viii) from description of ligands; Subst is equivalent leaving group as Subst from points vii) or viii) from description of ligand; n, m is independently 1 or 2, nn is 0 or 1;
by reaction with derivate of structure:
H₂N-Gr³
wherein Gr³ is group independently equivalent with (X^{t})C(Y^{t})(W^{t}) (t is 1 or 2 or 3) or Prot¹⁻³ from points i) to viii) from description of ligands;
under conditions of high dilution, template synthesis, reaction on solid phase, phase transfer catalysis, in aprotic polar solvents, with or without microwave irradiation, with or without presence of ultrasonic.

15. Method of preparation or synthesis of ligands of the general formula (1) as defined in claim 1 from protected intermediates of structure: 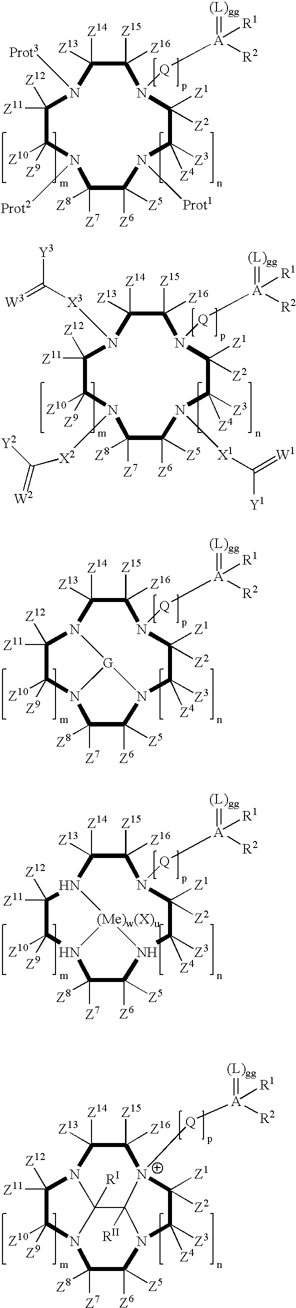 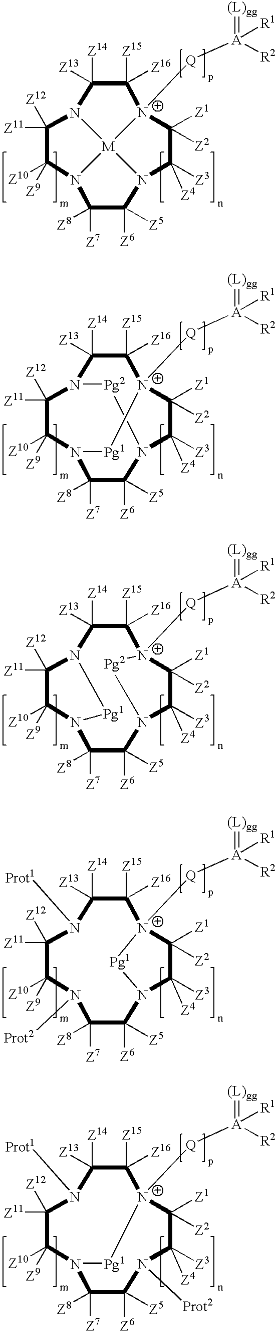
wherein Prot¹⁻³ is independently protective group (or electron pair with negative charge) especially of general structure: —CHO, —COR, —COOR, —CONRⁱRⁱⁱ, —SO₂R, —SR, —R, —SiRⁱRⁱⁱRⁱⁱⁱ, —PORⁱRⁱⁱ, —PSRⁱRⁱⁱ, —PO(ORⁱ)(ORⁱⁱ); protective groups Prot¹ and Prot² or Prot¹ and Prot¹ may be also connected to each other especially according to following general structure: —CRⁱRⁱⁱ—, —CO—, —COCO—, —CS—, —C(═NR)—, —COCRⁱRⁱⁱCO—, —CO—R—CO—, [—CRⁱRⁱⁱCRⁱRⁱⁱ]₂SO, [—CRⁱRⁱⁱCRⁱRⁱⁱ]₂SO₂, [—CRⁱRⁱⁱCRⁱRⁱⁱ]₂P(O)OR, [—CRⁱRⁱⁱCRⁱRⁱⁱ]₂NR, —PO(OR)—, —SiRⁱRⁱⁱ—, —SnRⁱRⁱⁱ— wherein Rⁱ⁻ⁱⁱⁱ are groups of the same type as R. Prot¹⁻³ is for example methanesulphonyl, 4-toluenesulphonyl, trifluoromethanesulphonyl, nitrobenzenesulphonyl, benzenesulphonyl, naphthalenesulphonyl, formyl, acetyl, benzoyl, phthaloyl, trifluoroacetyl, tert.-butoxycarbonyl (Boc), 9H-fluoren-9-yl-methoxycarbonyl (Fmoc), benzyloxycarbonyl (Z), ethoxycarbonyl (Eoc), methoxycarbonyl (Meoc), methoxybenzylcarbonyl (Moz), trityl, benzyl, benzhydryl, 4,4′-dimethoxytrityl, 4-methoxybenzoyl, ethandioyl, propandioyl, carbonyl, thiocarbonyl etc. wherein G is CZ¹⁻¹⁶ (include C⁺ as carbocation with Z¹⁻¹⁶ as anion), SiZ¹⁻¹⁶, SnZ¹⁻¹⁶, B, Al, P, As, PO, AsO, PS, AsS, AsZ¹⁻¹⁶, VZ¹⁻¹⁶, PZ¹⁻¹⁶;
G is CZ¹⁻¹⁶ (include C⁺ as carbocation with Z¹⁻¹⁶ as anion), SiZ¹⁻¹⁶, SnZ¹⁻¹⁶, B, Al, P, As, PO, AsO, PS, AsS, AsZ¹⁻¹⁶, VZ¹⁻¹⁶, PZ¹⁻¹⁶;
Me is metal (or ion), especially: Cu, Ni, Fe, Zn, Cr, Mo, V; X is ligand for example Cl, Br, OH, etc.; u is from 1 to 15; w is 1 or 2 or 3 or ½ or ⅔ or 3/2), or formates TiOZ¹⁻¹⁶, TiNZ¹⁻¹⁶, MoP, MoN;
wherein Rⁱ and Rⁱⁱ are groups of the same type as R; M is PR, P—SR, P-halogen, P—OR, silicon, carbon;
Pg¹⁻² is independently protective group especially of structure: CRⁱRⁱⁱ, SiRⁱRⁱⁱ, SnRⁱRⁱⁱ, CO, CS, C(═NR), PO(OR), PS(OR), PO(R), PS(R),
gg is 0 or 1
where molecular fragments -QₚA(L)_{gg}R¹R² and -QₚA(R¹)(R²)(R³)(R⁴) undergoes to transformations:
oxidation, by scheme:
-QₚA(L)HR¹+oxidant→-QₚA(L)R¹OH
-QₚA(R¹)(R²)+oxidant→-QₚA(L)R¹R²
wherein oxidant is atom or molecule with possibility to oxidation of -QₚA(L)HR¹ or -QₚA(R¹)(R²), e.g. oxygen, sulphur, hydrogen peroxide, hypochlorite, halogens, hexacyanoferrate(III), peroxodisulphate, peroxoborate, chromate and dichromate, permanganate, manganese(IV) dioxide etc.,
Addition, by Scheme:
-QₚA(L)HR¹+RⁱRⁱⁱC═CRⁱⁱⁱR^{iv→}-QₚA(L)(R²)[C(Rⁱ)(Rⁱⁱ)—C(Rⁱⁱⁱ)(R^{iv})(H)]
-QₚA(L)HR¹+RⁱRⁱⁱC═W^{1-3→}-QₚA(L)(R²)[C(Rⁱ)(Rⁱⁱ)(W′H)]
-QₚA(L)HR¹+RⁱRⁱⁱC═W¹⁻³+reductant→-QₚA(L)(R²)[C(Rⁱ)(Rⁱⁱ)(H)]
-QₚAH(R¹)(R²)(R³)+RⁱRⁱⁱC═CRⁱⁱⁱR^{iv→}-Qₚ[A(R⁴)(R⁵)(R⁶)]C(Rⁱ)(Rⁱⁱ)—C(Rⁱⁱⁱ)(R^{iv})(H)
-QₚAH(R¹)(R²)(R³)+RⁱRⁱⁱC═W^{1-3→}-Qₚ[A(R⁴)(R⁵)(R⁶)]C(Rⁱ)(Rⁱⁱ)(W′H)
-QₚAH(R¹)(R²)(R³)+RⁱRⁱⁱC═W¹⁻³+reductant→-Qₚ[A(R⁴)(R⁵)(R⁶)]C(Rⁱ)(Rⁱⁱ)(H)
-QₚAR¹R²+RⁱRⁱⁱC═CRⁱⁱⁱR^{iv→}-QₚA(L)(R³)[C(Rⁱ)(Rⁱⁱ)]—C(Rⁱⁱ)(R^{iv})(R⁴)
-QₚAR¹R²+RⁱRⁱⁱC═W^{1-3→}-QₚA(L)(R³)[C(Rⁱ)(Rⁱⁱ)(W′R⁴)]
-QₚA(L)HR¹+RⁱRⁱⁱC═W¹⁻³+Le-R^{2→}-QₚA(L)R³[C(Rⁱ)(Rⁱⁱ)(W′R⁴)]
-QₚAH(R¹)(R²)(R³)+RⁱRⁱⁱC═W¹⁻³+LeR^{4→}-QₚA(R⁵)(R⁶)(R⁷)[C(Rⁱ)(Rⁱⁱ)(W′R⁴)]
-QₚAR¹R²+RⁱRⁱⁱC═W¹⁻³+LeR^{3→}-QₚA(L)(R⁴)[C(Rⁱ)(Rⁱⁱ)(W′R³)]
-QₚA(L)R¹(CR²═CR³R⁴)+HW′R⁵→-QₚA(L)(R⁶){[C(R²)(H)]—[C(R³)(R⁴)(W′R⁵)]}
-QₚA(L)R¹(CR²═CR³R⁴)+AR⁵R⁶R⁷→-QₚA(L)(R⁸){[C(R²)(R⁵)]—[C(R³)(R⁴)(AR⁹R¹⁰)]}
-QₚA(L)R¹(CR²═CR³R⁴)+AR⁵R⁶R⁷→-QₚA(L)(R⁸){[C(R²)(R⁵)]—[C(R³)(R⁴)(A(L)R⁹R¹⁰)]}
-QₚA(L)R¹(CR²═CR³R⁴)+HAR⁵R⁶R⁷R⁸→-QₚA(L)(R⁸){[C(R²)(H)]—[C(R³)(R⁴)(AR⁹R¹⁰R¹¹R¹²)]}
QₚA(L)R¹(CR²═CR³R⁴)+HAR⁵R⁶R⁷R⁸→-QₚA(L)(R⁸){[C(R²)(H)]—[C(R³)(R⁴)(A(L)R⁹R¹⁰)]}
wherein Le is leaving group, especially of structure: —OR, —OH, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, N-benztriazolyl, 1-imidazolyl, succinimidyloxy, N-succinimidyl, N-phthalimidyl, N-phthalimidyloxy wherein Rⁱ⁻ⁱⁱⁱ are groups of the same type as R
wherein R^{i-iv} are groups of the same type as R, W′ is independently oxygen, sulphur, NH, NR⁶, A(L)R⁸, AR⁶R⁷R⁸, W¹⁻³;
R³⁻¹² are groups of the same type as R¹⁻²
Alkylation or Arylation by Scheme:
-QₚA(L)HR¹+Subst-R²→-QₚA(L)R¹R²
-QₚAR¹R²+Subst-R³→-QₚA(L)R⁴R³
wherein Subst is general leaving group, of structure: —OR, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, wherein Rⁱ⁻ⁱⁱⁱ are groups of the same type as R. (Subst is for example: hydroxyle, alcoxyl, aryloxyl, alkyl amine, N,N-dialkylamine, N-alkylamine, arylsulfonyloxy, tosyloxy, mesyloxy, triflyloxy, acetoxy, benzoyloxy, N-benztriazolyl, trialkylsilyloxy, hydrazine and N-substituted hydrazine, benzyloxycarbonyloxy, tert.-butyloxycarbonyloxy 1-imidazolyl, succinimidyloxy, N-succinimidyl, N-phthalimidyl, N-phthalimidyloxy, arylthio, thiole, S-alkylthiole etc.), or group which generates in situ or on introduced functional group R² with or without isolation cation or partial positive charge with capability to reaction (photochemically, thermically or electrochemically cleavable groups); R³⁻⁴ are groups of the same type as R¹⁻²
Substitution, by Scheme:
-QₚA(L)HR¹→-QₚA(L′)HR¹
-QₚA(L)HR¹→-QₚA(L′)HR²
-QₚA(L)HR¹→-QₚAR²R³
-QₚA(L)HR¹→-QₚAR¹R²
wherein R³ is group of the same type as R¹⁻²

16. Method of preparation or synthesis of ligands of the general formula (1) as defined in claim 1 from unitriprotected intermediates of structure: 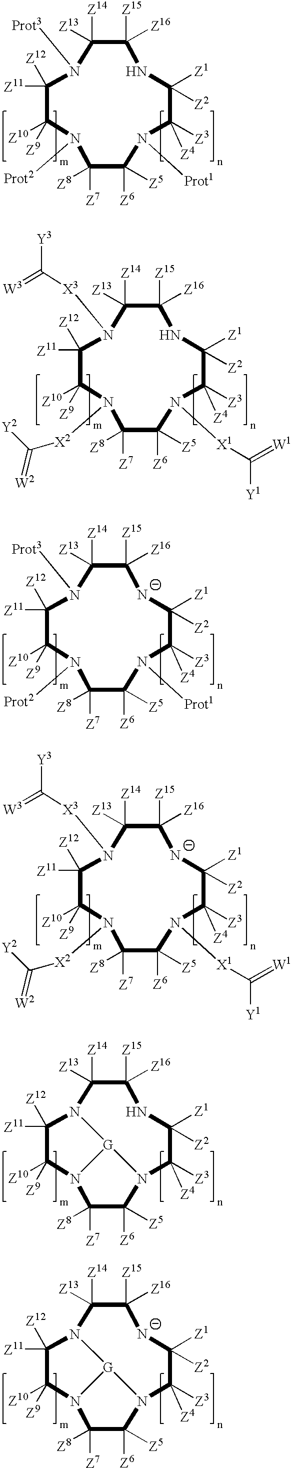 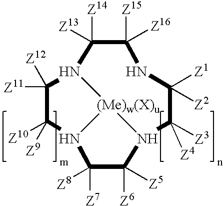
wherein G is CZ¹⁻¹⁶, (include C⁺ as carbocation with Z¹⁻¹⁶ as anion), SiZ¹⁻¹⁶, SnZ¹⁻¹⁶, B, Al, P, As, PO, AsO, PS, AsS, AsZ¹⁻¹⁶, VZ¹⁻¹⁶, PZ¹⁻¹⁶;
Me is metal (or ion), especially: Cu, Ni, Fe, Zn, Cr, Mo, V; X is ligand for example Cl, Br, OH, etc.; u is from 1 to 15; w is 1 or 2 or 3 or ½ or ⅔ or 3/2), or formates TiOZ¹⁻¹⁶, TiNZ¹⁻¹⁶, MoP, MoN;
wherein Prot¹⁻³ is independently protective group (or electron pair with negative charge) especially of general structure: —CHO, —COR, —COOR, —CONRⁱRⁱⁱ, —SO₂R, —SR, —R, —SiRⁱRⁱⁱRⁱⁱⁱ, —PORⁱRⁱⁱ, —PSRⁱRⁱⁱ, —PO(ORⁱ)(ORⁱⁱ); protective groups Prot¹ and Prot² or Prot¹ and Prot³ may be also connected to each other especially according to following general structure: —CRⁱRⁱⁱ—, —CO—, —COCO—, —CS—, —C(═NR)—, —COCRⁱRⁱⁱCO—, —CO—R—CO—, [—CRⁱRⁱⁱCRⁱRⁱⁱ]₂SO, [—CRⁱRⁱⁱCRⁱRⁱⁱ]₂SO₂, [—CRⁱRⁱⁱCRⁱRⁱⁱ]₂P(O)OR, [—CRⁱRⁱⁱCRⁱRⁱⁱ]₂NR, —PO(OR)—, —SiRⁱRⁱⁱ—, —SnRⁱRⁱⁱ— wherein Rⁱ⁻ⁱⁱⁱ are groups of the same type as R. Prot¹⁻³ is for example methanesulphonyl, 4-toluenesulphonyl, trifluoromethanesulphonyl, nitrobenzenesulphonyl, benzenesulphonyl, naphthalenesulphonyl, formyl, acetyl, benzoyl, phthaloyl, trifluoroacetyl, tert.-butoxycarbonyl (Boc), 9H-fluoren-9-yl-methoxycarbonyl (Fmoc), benzyloxycarbonyl (Z), ethoxycarbonyl (Eoc), methoxycarbonyl (Meoc), methoxybenzylcarbonyl (Moz), trityl, benzyl, benzhydryl, 4,4-dimethoxytrityl, 4-methoxybenzoyl, ethandioyl, propandioyl, carbonyl, thiocarbonyl etc.
by condensation with A(L)(R¹)(R²)(R³), HA(L)(R¹)(R²), A(R¹)(R²)(R³), HA(R¹)(R²), HA(R¹)(R²)(R³)(R⁴) of structure: 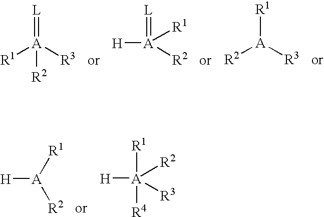
wherein R³ and R⁴ are groups of the same type as R¹⁻²,
with methylene or substituted methylene reactive group structure of aldehyde (e.g. formaldehyde, acetaldehyde, benzaldehyde, 4-N,N-dimethylaminobenzaldehyde, nitrobenzaldehyde, 2-chlorobenzaldehyde, anisaldehyde etc.), aldehyde acetals or semiacetals (e.g. formaldehyde dimethylacetal), chloromethylethers (e.g. chloromethylmethylether), 1,1,1-trialkoxyalkane, diazomethane or C-substituted diazomethanes
under conditions of general condensation: especially under conditions of azeotropic water off distillation, phase-transfer catalysis, in aprotic polar solvents or its mixtures (as dimethylformamide or dimethylacetamide or acetonitrile, dimethylsulphoxide or sulpholane or hexamethylphosphortriamide), in micellar medium, in solidphase (for example bonded N₄G⁻ on anex), with or without microwave irradiation, with or without ultrasonic irradiation, under conditions of high pressure (for example in autoclave), in aqueous phase in presence of pH-buffer, in milieu of water-free solvents with or without presence of base (for example: amines, aldimines, carbonates, fluorides, thioethers), enzymatic catalysis, in presence of dehydrating agent or agent reacting with protogenic product reaction or in presence of Lewis acid (e.g. ZnCl₂, BF₃, Et₂O, SiCl₄) etc.
and if needed, by next partially or full cleaving of G or (Me)_{w}(X)ᵤ. Both the steps can be solved as one-step reaction or separately.

17. Method of preparation or synthesis of ligands of the general formula (1) as defined in claim 1 from vinyl-triprotected intermediates: 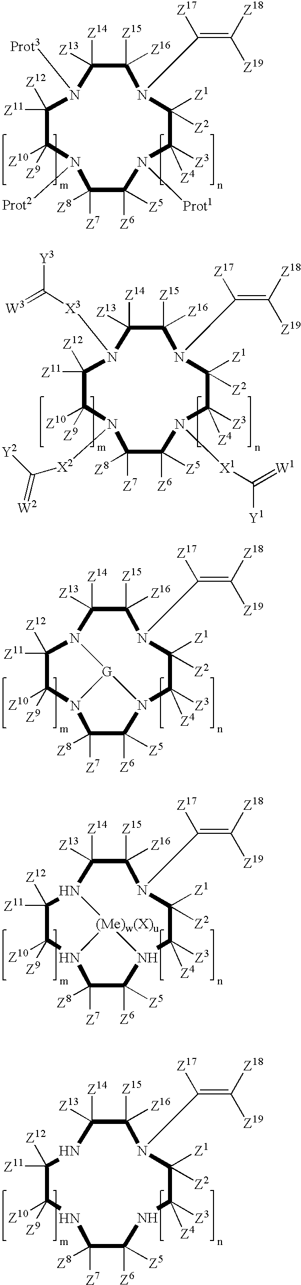
wherein
G is CZ¹⁻¹⁶ (include C⁺ as carbocation with Z¹⁻¹⁶ as anion), SiZ¹⁻¹⁶, SnZ¹⁻¹⁶, B, Al, P, As, PO, AsO, PS, AsS, AsZ¹⁻¹⁶, VZ¹⁻¹⁶, PZ¹⁻¹⁶;
Me is metal (or ion), especially: Cu, Ni, Fe, Zn, Cr, Mo, V; X is ligand for example Cl, Br, OH, etc.; u is from 1 to 15; w is 1 or 2 or 3 or ½ or ⅔ or 3/2), or formates TiOZ¹⁻¹⁶, TiNZ¹⁻¹⁶, MoP, MoN;
Z^{17-Z19} are groups of the same type as Z¹⁻¹⁶
Prot¹⁻³ is independently protective group (or electron pair with negative charge) especially of general structure: —CHO, —COR, —COOR, —CONRⁱRⁱⁱ, —SO₂R, —SR, —R, —SiRⁱRⁱⁱRⁱⁱⁱ, —PORⁱRⁱⁱ, —PSRⁱRⁱⁱ, —PO(ORⁱ)(ORⁱⁱ); protective groups Prot¹ and Prot² or Prot¹ and Prot³ may be also connected to each other especially according to following general structure: —CRⁱRⁱⁱ—, —CO—, —COCO—, —CS—, —C(═NR)—, —COCRⁱRⁱⁱCO—, —CO—R—CO—, [—CRⁱRⁱⁱCRⁱRⁱⁱ]₂SO, [—CRⁱRⁱⁱCRⁱRⁱⁱ]₂SO₂, [—CRⁱRⁱⁱCRⁱRⁱⁱ]₂P(O)OR, [—CRⁱRⁱⁱCRⁱRⁱⁱ]₂NR, —PO(OR)—, —SiRⁱRⁱⁱ—, —SnRⁱRⁱⁱ— wherein Rⁱ⁻ⁱⁱⁱ are groups of the same type as R. Prot¹⁻³ is for example methanesulphonyl, 4-toluenesulphonyl, trifluoromethanesulphonyl, nitrobenzenesulphonyl, benzenesulphonyl, naphthalenesulphonyl, formyl, acetyl, benzoyl, phthaloyl, trifluoroacetyl, tert,-butoxycarbonyl (Boo), 9H-fluoren-9-yl-methoxycarbonyl (Fmoc), benzyloxycarbonyl (Z), ethoxycarbonyl (Eoc), methoxycarbonyl (Meoc), methoxybenzylcarbonyl (Moz), trityl, benzyl, benzhydryl, 4,4′-dimethoxytrityl, 4-methoxybenzoyl, ethandioyl, propandioyl, carbonyl, thiocarbonyl etc.
by reaction (e.g. addition) with precursors or their mixture of structure: 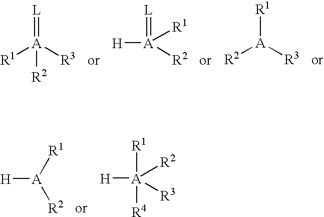
wherein R³ and R⁴ are groups of the same type as R¹⁻². Especially can be used: alkylphosphinic acid, arylphosphinic acid, trialkylphosphites, triarylphosphites, trialkylphosphines, triarylphosphines, dialkylphosphinates, diarylphosphinates, alkylarylphosphinates, dialkylarylphosphites, alkyldiarylphosphites, phosphinic acid, alkylarsenic(III) acid, arylarsenic(III) acid, trialkylarsenic(III), triarylarsenic(III), etc.
under conditions of general addition: especially carried out under high-pressure (for example in autoclave), microwave irradiation, under reflux in high boiling solvents, in micellar systems, in solid phase, under cryogenic conditions, under phase transfer catalysis, etc.

18. Method of preparation or synthesis of triprotected intermediates for synthesis ligands as defined in claims 3 and 4 of the structure: 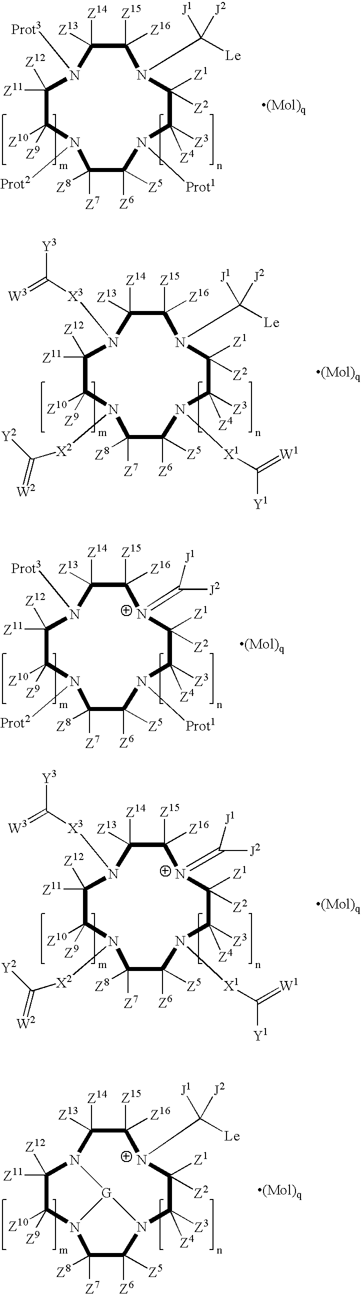 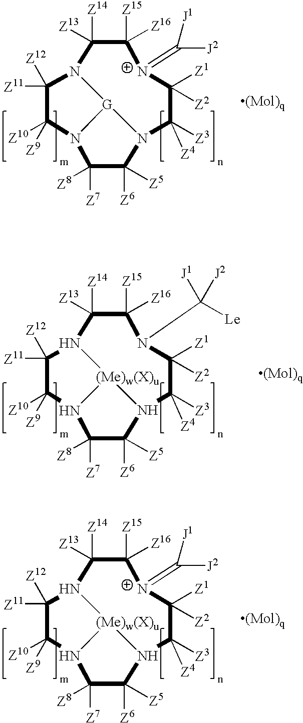
wherein:
A is phosphorus or arsenic;
Z¹⁻¹⁶ is independently radical of hydrogen; chlorine; bromine; fluorine; iodine; nitro or nitrosogroup; sulphogroup; substituted or unsubstituted aliphatic or alicyclic or cyclic alkyl with or without one or more double or triple bonds and with or without heteroatoms; substituted or unsubstituted aromatic radical or its aryloxyderivate; hydroxyle; alcoxyle; S-substituted or S-unsubstituted thiole; substituted or unsubstituted amine; Z¹⁻¹⁶ also can constitute independently carbonyle and general functional derivates of carbonyle as oxime, hydrazone etc. but especially N-substituted or unsubstituted carboimidyle; thiocarbonyle; condensed substituted or unsubstituted benzoderivate;
n, m is independently 1 or 2;
X¹⁻³ is independently methylene or ethylene substituted as Z¹⁻¹⁶ especially with or without heteroatoms and multiple bonds; carbonyle; N-substituted or unsubstituted carboimidyle; thiocarbonyle;
Y¹⁻³ is independently methyl substituted as Z¹⁻¹⁶; hydroxyle; O-substituted hydroxyle with Z¹⁻¹⁶; S-substituted thiole; substituted or unsubstituted amine; hydroxylate or thiolate of metal cations or organic cations (for example: Na, Li, K, Rb, Cs, Ca, Mg, Al, Zn, Mn, Cr, Mo, ⁶⁴Cu, ⁶⁷Cu, ⁶⁷Ga, ⁹⁰Y, ¹¹¹In, ¹⁵³Sm, ¹⁶⁶Ho, ¹⁷⁷Lu, ²⁰¹Tl, ²¹²Bi, ammonium, primary, secondary, tertiary and quarternary alkyl and arylammonium, sulphonium and phosphonium salts and their combinations); Y¹⁻³ can constitute independently substituted hydroxylamine of formula: 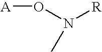
wherein A is independently methyl substituted as Z¹⁻¹⁶; metal cation or organic cation (for example: Na, Li, K, Rb, Cs, Ca, Mg, Ai, Zn, Mn, Cr, Mo, ¹⁴Cu, ⁶⁷Cu, ⁶⁷Ga, ⁹⁰Y, ¹¹¹In, ¹⁵³Sm, ¹⁶⁶Ho, ¹⁷⁷Lu, ²⁰¹Tl, ²¹²Bi, ammonium, primary, secondary, tertiary and quarternary alkyl and arylammonium, sulphonium and phosphonium salts and their combinations);
R is independently radical of hydrogen; substituted or unsubstituted aliphatic or alicyclic or cyclic alkyl with or without one or more double or triple bonds and with or without heteroatoms; substituted or unsubstituted aromatic radical;
R¹⁻² is independently hydrogen; halogene; substituted or unsubstituted aliphatic or alicyclic or cyclic alkyl with or without one or more double or triple bonds and with or without heteroatoms; substituted or unsubstituted aromatic radical or its aryloxyderivate; hydroxyle; alcoxyle; thiole; thioalcoxyle; substituted or unsubstituted amine; trialkylsilyl; trialkylsilyloxy, triarylsilyl; triarylsilyloxy; hydroxylate or thiolate of metal cations or organic cations (for example: Na, Li, K, Rb, Cs, Ca, Mg, Al, Zn, Mn, Cr, Mo, ⁶⁴Cu, ⁶⁷Cu ⁶⁷Ga, ⁹⁰Y, ¹¹¹In, ¹⁵³Sm, ¹⁶⁶Ho, ¹⁷⁷Lu, ²⁰¹Tl, ²¹²Bi, ammonium, primary, secondary, tertiary and quarternary alkyl and arylammonium, sulphonium and phosphonium salts and their combinations);
W¹⁻³ is independently oxygen, sulphur, N-substituted or unsubstituted imidyl;
G is CZ¹⁻¹⁶ (include C⁺ as carbocation with Z¹⁻¹⁶ as anion), SiZ¹⁻¹⁶, SnZ¹⁻¹⁶, B, Al, P, As, PO, AsO, PS, AsS, AsZ¹⁻¹⁶, VZ¹⁻¹⁶, PZ¹⁻¹⁶;
Me is metal (or ion), especially: Cu, Ni, Fe, Zn, Cr, Mo, V; X is ligand for example Cl, Br, OH, etc.; u is from 1 to 15; w is 1 or 2 or 3 or ½ or ⅔ or 3/2), or formates TiOZ¹⁻¹⁶, TiNZ¹⁻¹⁶, MoP, MoN;
J¹⁻² is group (substituent, fragment) of the same type as Z¹⁻¹⁶;
Le is leaving group, especially of structure: —OR, —OH, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, —W¹⁻³H, —W¹⁻³R, N-benztriazolyl, 1-imidazolyl, succinimidyloxy, N-succinimidyl, N-phthalimidyl, N-phthalimidyloxy wherein Rⁱ⁻ⁱⁱⁱ are groups of the same type as R;
Prot¹⁻³ is independently protective group (or electron pair with negative charge) especially of general structure: —CHO, —COR, —COOR, —CONRⁱRⁱⁱ, —SO₂R, —SR, —R, —SiRⁱRⁱⁱRⁱⁱⁱ, —PORⁱRⁱⁱ, —PSRⁱRⁱⁱ, —PO(ORⁱ)(ORⁱ); protective groups Prot¹ and Prot² or Prot¹ and Prot³ may be also connected to each other especially according to following general structure: —CRⁱRⁱⁱ—, —CO—, —COCO—, —CS—, —C(═NR)—, —COCRⁱRⁱⁱCO—, —CO—R—CO—, [—CRⁱRⁱⁱCRⁱRⁱⁱ]₂SO, [—CRⁱRⁱⁱCRⁱRⁱⁱ]₂SO₂, [—CRⁱRⁱⁱCRⁱRⁱⁱ]₂P(O)OR, [—CRⁱRⁱⁱCRⁱRⁱⁱ]₂NR, —PO(OR)—, —SiRⁱRⁱⁱ—, —SnRⁱRⁱⁱ— wherein Rⁱ⁻ⁱⁱⁱ are groups of the same type as R. Prot¹⁻³ is for example methanesulphonyl, 4-toluenesulphonyl, trifluoromethanesulphonyl, nitrobenzenesulphonyl, benzenesulphonyl, naphthalenesulphonyl, formyl, acetyl, benzoyl, phthaloyl, trifluoroacetyl, tert.-butoxycarbonyl (Boc), 9H-fluoren-9-yl-methoxycarbonyl (Fmoc), benzyloxycarbonyl (Z), ethoxycarbonyl (Eoc), methoxycarbonyl (Meoc), methoxybenzylcarbonyl (Moz), trityl, benzyl, benzhydryl, 4,4′-dimethoxytrityl, 4-methoxybenzoyl, ethandioyl, propandioyl, carbonyl, thiocarbonyl etc.
Mol is protogenic acid (for example: mineral acid, substituted or unsubstituted carboxylic, sulphonic, phosphonic and phosphinic acid) or protophilic base (for example: pyridine, tetrahydrofurane, triethylphosphine) or Lewis acid (for example: BF₃, ZnCl₂, AlCl₃, FeBr₃) or neutral molecule bonded as e.g. in molecular cluster or associate (e.g. chloroform, toluene, cyclodextrine, calix[8]arene, polyethyleneglycole 800), q is from 0 to 10 or ½ or ⅔ or ¾, 4/3, 3/2;
by usage of one or more of these synthetic methods or synthetic routes anywhere in all synthetic approach in anyone from all used steps for synthesis of triprotected intermediates as described in xviii.
a) by reaction of protected or unprotected macrocyclic tetramine or its salt or its appropriate anion (structure b1 and b2) with reactive intermediates of the type: Subst-[C(J¹)(J²)]-Le, (J¹)(J²)C═W¹⁻³, (J¹)(J²)C═W¹⁻³ and HW¹⁻³R mixture, according to schemes B1-B3: 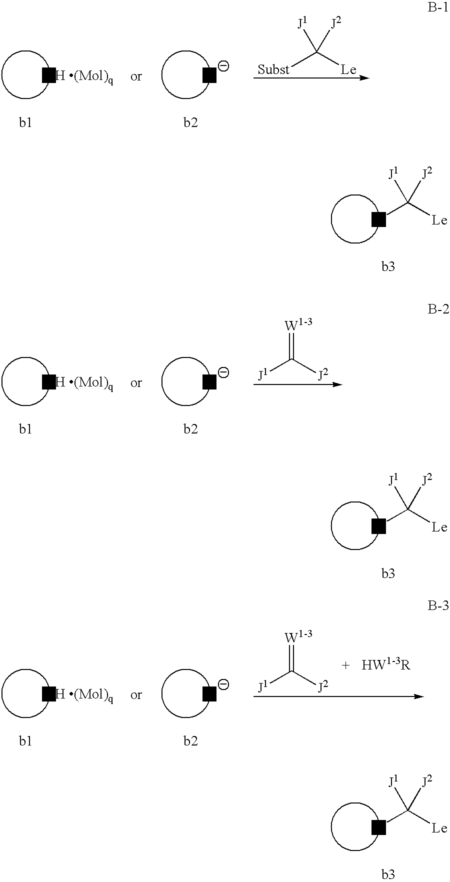
wherein Subst is general leaving group, of structure: —OR, —O⁺RⁱRⁱⁱ, —OSiRⁱRⁱⁱRⁱⁱⁱ, —OCOR, —OCONRⁱRⁱⁱ, —OSO₂R, —ON(CORⁱ)(CORⁱⁱ), —NRⁱRⁱⁱ, —(NRⁱRⁱⁱRⁱⁱⁱ)⁺, —N(CORⁱ)(CORⁱⁱ), —N(SO₂Rⁱ)(SO₂Rⁱⁱ), —NSO₂R, -halogene, —NRⁱNRⁱⁱRⁱⁱⁱ, —SR, —SO₃H or —SO₂Y¹⁻³, wherein Rⁱ⁻ⁱⁱⁱ are groups of the same type as R. (Subst is for example: hydroxyle, alcoxyl, aryloxyl, alkyl amine, N,N-dialkylamine, N-alkylamine, arylsulfonyloxy, tosyloxy, mesyloxy, triflyloxy, acetoxy, benzoyloxy, N-benztriazolyl, trialkylsilyloxy, hydrazine and N-substituted hydrazine, benzyloxycarbonyloxy, tert.-butyloxycarbonyloxy, 1-imidazolyl, succinimidyloxy, N-succinimidyl, N-phthalimidyl, N-phthalimidyloxy, arylthio, thiole, S-alkylthiole etc.),
wherein symbol 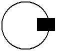
is chain 1 or chain 2 or chain 3 or chain 4 of structure: 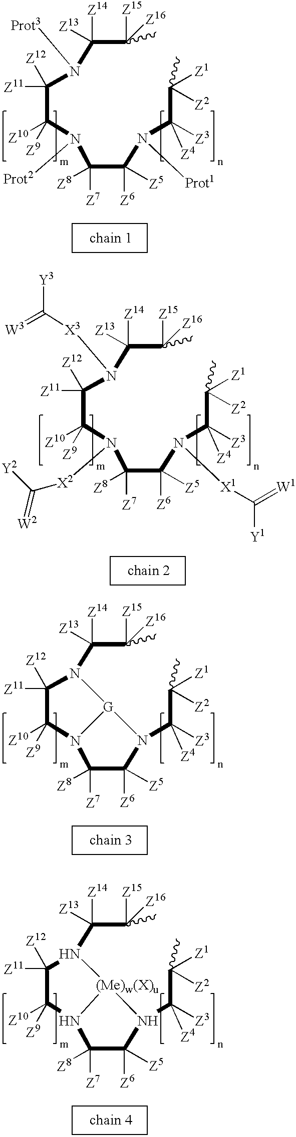
under conditions of general nucleofilic substitution: especially under conditions of phase-transfer catalysis, in aprotic polar solvents or its mixtures (as dimethylformamide or dimethylacetamide acetonitrile, dimethylsulphoxide or sulpholane or hexamethylphosphortriamide), in micellar medium, in solidphase (for example bonded N₄G⁻ on anex), with or without microwave irradiation, with or without ultrasonic irradiation, under conditions of high pressure (for example in autoclave), in aqueous phase in presence of pH-buffer, in milieu of water-free solvents with or without presence of base (for example: amines, aldimines, anex, carbonates, fluorides, thioethers), enzymatic catalysis, in presence of dehydrating agent or agent reacting with protogenic product reaction or in presence of Lewis acid as catalysators (for example ZnCl₂, BF₃.Et₂O, SiCl₄) etc.
or
b) by reaction of intermediate b3 with an agent eliminating Le⁻ anion by scheme B4. [chemical structure]
